(19) United States
(12) Patent Application Publication
(10) Pub. No.: US 2025/0195678 A1
(43) Pub. Date: Jun. 19, 2025

(54) B-LYMPHOCYTE SPECIFIC AMATOXIN ANTIBODY CONJUGATES

(71) Applicant: Heidelberg Pharma Research GmbH, Ladenburg (DE)

(72) Inventors: Torsten Hechler, Ladenburg (DE); Andreas Pahl, Ladenburg (DE); Michael Kulke, Ladenburg (DE); Christoph Müller, Ladenburg (DE)

(73) Assignee: Heidelberg Pharma Research GmbH, Ladenburg (DE)

(21) Appl. No.: 17/769,183

(22) PCT Filed: Oct. 15, 2020
(86) PCT No.: PCT/EP2020/078979

§ 371 Date: Apr. 14, 2022

(30) Foreign Application Priority Data
Oct. 15, 2019 (EP) 19203400.7

(51) Int. Cl. A61K 47/68; A61P 35/00

(52) U.S. Cl. CPC A61K 47/6831; A61K 47/6867; A61K 47/6889; A61P 35/00

(57) ABSTRACT

The present application relates to a conjugate comprising an amatoxin, a target-binding moiety wherein the target is CD20, i.e., a CD20-binding moiety, and optionally a linker linking said amatoxin and said CD20-binding moiety. The invention further relates to the synthesis of said conjugate. In addition, the invention relates to a pharmaceutical composition comprising such conjugate, particularly for use in the treatment of B-cell and/or lymphoma associated diseases and/or malignancies.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 20,673 bytes ASCII (Text) file named “759891_Replacement_ST25.txt” created Dec. 28, 2023.

### FIELD OF THE INVENTION

The present application relates to a conjugate comprising an amatoxin, a target-binding moiety wherein the target is CD20, i.e., a CD20-binding moiety, and optionally a linker linking said amatoxin and said CD20-binding moiety. The invention further relates to the synthesis of said conjugate. In addition, the invention relates to a pharmaceutical composition comprising such conjugate, particularly for use in the treatment of B-cell and/or lymphoma associated diseases and/or malignancies.

### BACKGROUND

CD20 is a cell-surface, membrane-embedded 35-37 kDa non-glycosylated phosphoprotein that is characteristic for certain B-cell precursors (pre-B lymphocytes) and mature B lymphocytes. The CD20 antigen is neither expressed on plasma cells, nor on hematopoietic stem cells and early pro-B lymphocytes. No natural CD20 ligand has been identified so far. CD20 plays a role in the development, growth and differentiation of B-cells into plasma cells and enables optimal B-cell immune response, specifically against T-independent antigens. Some data have indicated that CD20 could function as a Ca²⁺ membrane channel that sustains intracellular Ca²⁺ concentration and allows the activation of B cells (Winiarska et al, 2007).

The CD20 antigen is a member of the membrane-spanning 4A protein family; its structure consists of 4 membrane-spanning domains with both amino and carboxy termini located within the cytoplasm (hence, CD20 is also called MS4A1—membrane spanning 4 domain subfamily A, member 1). CD20 has two extracellular loops. The smaller one (a segment of seven amino acids between the first and second transmembrane regions) probably does not extent beyond the cellular membrane; this loop is identical in every member of the MS4A family. The bigger loop, a segment of 43 amino acids between the third and fourth transmembrane regions has a disulfide bond and is recognized by the majority of anti-CD20 antibodies. No tyrosine residues or recognized signal transduction motifs occur in any of the cytoplasmic regions of the CD20 molecule, although there are a number of consensus sites for serine and threonine phosphorylation.

CD20 may exist as dimers and tetramers in complex with at least one additional protein component. The CD20 protein has been reported to be closely associated with the transmembrane adapter protein p75/80 (also named C-terminal src kinase-binding protein Cbp), CD40 and major histocompatibility complex class II proteins (MHC II). CD20 antigen undergoes conformational changes during B lymphocyte diferentiation, and at least two conformational isoforms of CD20 exist (Winiarska et al, 2007).

Certain characteristics have made the CD20 antigen an appealing target for monoclonal antibody (mAb) therapy. The CD20 antigen seems to be one of the most stable lymphocyte antigens. It does not circulate in the plasma as free protein that could competitively inhibit monoclonal antibodies from binding to lymphoma cells, does not shed from the surface of CD20-positive cells after antibody binding, and in the large majority of in vivo and in vitro studies no internalization of the CD20 surface molecule or down-regulation in CD20 expression was detected.

The anti-CD20, B-cell-specific, chimeric monoclonal antibody rituximab has been the first monoclonal antibody approved by regulatory authorities for treatment of various cancer and autoimmune diseases such as non-Hodgkin's B-cell lymphoma, chronic lymphocytic leukemia and rheumatoid arthritis. Rituximab has been demonstrated to promote antibody-dependent cell-mediated cytotoxicity (ADCC) and complement-dependent cytotoxicity (CDC) of human lymphoid cell lines expressing CD20 (Taylor and Lindorfer, 2008), as well as to exert direct effects on cell signaling pathways and cell membranes following CD20 binding. A large number of events that are affected by rituximab binding have been identified, including lipid raft modifications, kinase and caspase activation, and effects on transcription factors (Weiner 2010; Bezombes et al, 2011).

Beside rituximab, other anti-CD20 antibodies have been described, including ibritumomab, tositumomab (both conjugated with radioisotope), ofatumumab, ocrelizumab, obinutuzumab, and ublituximab, which are all active agents intended for the treatment of B cell lymphomas, leukemias, and B cell-mediated autoimmune diseases (Falchi et al, 2018).

Rituximab has been approved in Europe for adults under the trade name MabThera (Roche) for the following indications: non-Hodgkin's lymphoma, NHL (treatment of previously untreated patients with stage III-IV follicular lymphoma in combination with chemotherapy; maintenance therapy for the treatment of follicular lymphoma patients responding to induction therapy; monotherapy for treatment of patients with stage III-IV follicular lymphoma who are chemoresistant or are in their second or subsequent relapse after chemotherapy; treatment of patients with CD20-positive diffuse large B cell non-Hodgkin's lymphoma in combination with CHOP (cyclophosphamide, doxorubicin, vincristine, prednisolone) chemotherapy; chronic lymphocytic leukaemia, CLL (in combination with chemotherapy for the treatment of patients with previously untreated and relapsed/refractory CLL); rheumatoid arthritis (in combination with methotrexate for the treatment of adult patients with severe active rheumatoid arthritis who have had an inadequate response or intolerance to other disease-modifying anti-rheumatic drugs (DMARD) including one or more tumor necrosis factor (TNF) inhibitor therapies); granulomatosis with polyangiitis and microscopic polyangiitis (in combination with glucocorticoids for the treatment of adult patients with severe, active granulomatosis with polyangiitis (Wegener's) (GPA) and microscopic polyangiitis (MPA)); and pemphigus vulgaris (for the treatment of patients with moderate to severe pemphigus vulgaris) (MabThera, Summary of Product Characteristics).

Attempts to use anti-CD20 antibodies for construction of antibody-drug conjugates (ADCs), however, have been of limited success.

Negative results with toxin conjugates of anti-CD20 antibodies were reported by Lambert et al. (1985) who found that immunotoxins containing an antibody anti-B1, directed against CD20, showed no cytotoxicity, in contrast to immunotoxins comprising various other monoclonal antibodies of the IgG class that were reactive with three other antigens on human lymphoid cells. The immunotoxins used in this study were comprising the ribosome-inactivating proteins gelonin or the three known pokeweed antiviral proteins.

As disclosed by A. G. Polson in Phillips (2013), CD20 differs from other B-cell surface proteins used as targets in that it was found to be very poorly internalized upon antibody binding, and therefore to be less suited as an ADC target. Early studies had already concluded that because anti-CD20 antibodies were regarded as non-internalizing, such conjugates were not considered to be promising. The finding that CD20 is a non-internalizing or poorly, insufficiently internalizing antigen has been widely confirmed in the literature (Press et al, 1989; Vangeepuram et al, 1997; Winiarska et al, 2007; Kim and Kim, 2015; Staudacher and Brown, 2017).

DiJoseph et al. (2007) concluded from their study on non-Hodgkin's B-cell lymphoma cells that due to the lack of internalization of antibody-bound CD20, their amide-linked conjugate of rituximab did not deliver the toxin calicheamicin in the intracellular compartments to bring about its cytotoxic activity.

Other studies disclosed that ADCs comprising Anti-CD20 antibodies as targeting moieties, N(2′)-deacetyl-N(2′)-(3-mercapto-1-oxopropyl)-maytansine (DM1) as toxin, and uncleavable linker were ineffective on CD20-positive target cells, whereas respective constructs with cleavable linker yielded some cytotoxic effect on such cells (Polson et al, 2009). Law et al. (2004) found different effects of CD20-targeted ADCs depending on the toxin used: Anti-CD20 antibody conjugated to doxorubicin (Dox) failed to deliver drug or to demonstrate any anti-tumor activity, while anti-CD20 antibody-drug conjugates employing the anti-mitotic agent monomethyl auristatin E (MMAE) exerted potent anti-tumor activity.

Furthermore, in addition to the nature of the toxin used and the nature of the linker employed for conjugation of the target-binding moiety to such toxin, the specific characteristics of the particular anti-CD20 antibody appeared to be relevant for determining the therapeutic efficacy of such antibody conjugates according to recent studies. Whereas so-called “type-II” CD20-specific antibodies have been shown to be poorly internalized by CD20-positive target cells, other so-called “type-I” CD20-specific antibodies have been found to be internalized and degraded to some extent, depending on the level of expression of activatory and inhibitory FcγR on target cells with which they interact. In particular, the inhibitory FcR, FcγRIIb, expressed on certain types of B-cell malignancies, has been described to play a critical role in this context. The mechanisms underlying the differential FcγR-mediated internalization response of type-I and type-II anti-CD20 monoclonal antibodies have not yet defined in detail (Boross and Leussen, 2012; Dransfield 2014; Vaughan et al. 2014).

Internalization of the target is highly desirable in the context of using antibody-drug conjugates (ADCs) for cytotoxic cancer therapy (e.g., Boross and Leussen, 2012). Internalization of ADCs upon binding to the target is often necessary for optimal efficacy of the ADC, because cytotoxic payloads typically act on intracellular targets (Kim and Kim, 2015). This can in particular be considered holding true for amanitin-based ADCs comprising amanitins and their derivatives (“amatoxins”), because these amatoxins are less hydrophobic than other toxin molecules that have been employed for production of ADCs, and hence less capable of permeating cell membranes as compared to diffusible drugs like, e.g., MMAE (Staudacher and Brown, 2017). In view of respective prejudice in literature and conflicting results of different studies with regard to internalization and cytotoxic effect of anti-CD20 antibodies and ADCs employing the same, as described above, inventors of the present application did not have any expectation of success in terms of a significant cytotoxic effect when using anti-CD20 antibodies in the context of antibody amatoxin conjugates. Amanitin or amanitin analogs or derivatives had not been used for synthesis and evaluation of ADCs comprising CD20 target binding moieties ever before.

Surprisingly, the inventors found that amatoxin-based ADCs comprising anti-CD20 antibody, either with non-cleavable or cleavable linker linking the anti-CD20 antibody, or antibody fragment or antibody derivative, to the amatoxin, exerted significant cytotoxic effect on CD20-positive target cells in vitro and in vivo. These results were unexpected, particularly for amatoxin-based ADCs comprising non-cleavable linkers, as they are considered to require intracellular degradation within lysosomal compartments for release of active toxin molecules.

### SUMMARY OF THE INVENTION

In view of the prior art, it was hence one object of the present invention to provide conjugates comprising a target binding moiety binding to CD20, at least one amatoxin, and optionally at least one linker connecting said target binding moiety with said at least one toxin, that mediate cytotoxic effects in target cells, as described in the present application.

It was one further object of the present invention to provide conjugates comprising a target binding moiety binding to CD20, at least one amatoxin, and optionally at least one linker, wherein said target binding moieties are antibodies, or antigen-binding fragments thereof, or antigen-binding derivatives thereof, or antibody-like proteins, that specifically bind to CD20.

It was one further object of the present invention to provide a pharmaceutical composition comprising such conjugates.

It was one further object of the present invention to provide compounds for use in methods for treatment of cancer and autoimmune diseases.

It was one further object of the present invention to provide conjugates comprising a target binding moiety binding to CD20, at least one amatoxin, and optionally at least one linker, for use in the treatment of B lymphocyte-associated malignancies or B cell-mediated autoimmune diseases, in particular for use in the treatment of non-Hodgkin's lymphoma, follicular lymphoma, diffuse large B cell non-Hodgkin's lymphoma, chronic lymphocytic leukaemia, Richter syndrome, rheumatoid arthritis, granulomatosis with polyangiitis and microscopic polyangiitis and pemphigus vulgaris.

It was surprisingly found that said conjugates comprising a target binding moiety binding to CD20, at least one amatoxin, and optionally at least one linker, in particular amatoxin-based ADCs comprising anti-CD20 antibody, either with non-cleavable or cleavable linker connecting the anti-CD20 antibody, or antibody fragment or antibody derivative, to the amatoxin, exerted significant cytotoxic effects on CD20-positive target cells in vitro and in vivo. These results were unexpected, particularly for amatoxin-based ADCs comprising non-cleavable linkers, as they are considered to require intracellular degradation within lysosomal compartments for release of active toxin molecules.

These and further objects are met with methods and means according to the independent claims of the present invention. The dependent claims are related to specific embodiments.

The invention and general advantages of its features will be discussed in detail below.

### DESCRIPTION OF THE FIGURES

FIG. 1. Structural formulae of various amatoxins. The numbers in bold type (1 to 8) designate the standard numbering of the eight amino acids forming the amatoxin. The standard designations of the atoms in amino acids 1, 3, and 4 are also shown (Greek letters a to Y, Greek letters a to 8, and numbers from 1′ to 7′, respectively).

FIG. 2. Binding of the anti-CD20 monoclonal antibody rituximab to the CD20-positive human chronic B-cell leukemia cell lines (A) MEC-1 and (B) MEC-2 and to the human Burkitt lymphoma cell line (C) Raji as demonstrated by FACS analysis.

FIG. 3. Results of cytotoxicity studies on CD20-positive MEC-1 cells in a BrdU assay after incubation at 72 hours.

FIG. 4. Results of cytotoxicity studies on CD20-negative SK-Hep-1 cells in a BrdU assay after incubation at 72 hours.

FIG. 5. Results of cytotoxicity studies (A) on non-stimulated PBMCs and (B) on CD20-enriched non-stimulated PBMCs using anti-CD20 antibody-(Rtx-30.0643) and anti-EGFR-antibody-(Her-30.0643, Pan-30.0643) amatoxin conjugates in a WST-1 assay.

FIG. 6. Results of cytotoxicity studies on CD20-positive MEC-1 cells in a BrdU assay after incubation at 72 hours using the rituximab-amatoxin conjugate Rtx-30.0643, the rituximabF (ab′) 2 fragment amatoxin conjugate Rtx-F(ab′)₂-30.0643, and unconjugated rituximab.

FIG. 7. Results of in vivo efficacy study using the rituximab-amatoxin conjugate Rtx-30.0643 in the P493-6 xenograft model (human Burkitt's Lymphoma, B-cell Lymphoma) in SCID beige-based mice.

FIG. 8. Results of an exploratory toxicity study in Cynomolgus monkeys (Macaca fascicularis) using rituximab-amatoxin conjugate Rtx-30.0643 (with a payload of 3.2 amanitin moieties per IgG molecule) showing significant B cell depletion; unconjugated rituximab was used as a reference (control).

FIG. 9. Results of an exploratory toxicity study in Cynomolgus monkeys (Macaca fascicularis) using rituximab-amatoxin conjugate Rtx-30.0643 (with a payload of 3.2 amanitin moieties per IgG molecule) showing no significant changes in body weight in the two study groups.

FIG. 10. Results of cytotoxicity studies on CD20-positive MEC-1 cells in a BrdU assay in vitro using the rituximab amatoxin conjugates Rtx-DSC-30.0353, Rtx-DSC-30.0354, and Rtx-DSC-30.0355.

FIG. 11. Results of cytotoxicity studies on CD20-positive MEC-1 cells in a WST-1 assay using the rituximab amatoxin conjugates (A) Rtx-30.0643, Rtx-30.748, Rtx-30.1214, (B) Rtx-30.1215, Rtx-30.1216, Rtx-30.1217 and (C) Rtx-30.1218.

FIG. 12. Results of (A) SDS-PAGE analysis and (B) Western blots (developed by use of anti-amanitin antibodies) of rituximab amatoxin conjugates Rtx-30.1699 and Rtx-30.2115, respectively.

FIG. 13. SEC HPLC analyses of conjugates Rtx-30.1699 (upper panel) and Rtx-30.2115 (lower panel), respectively.

FIG. 14. Results of cytotoxicity studies on the human chronic B-cell leukemia cell lines (A) MEC-1 and (B) MEC-2, respectively, using the rituximab-amatoxin conjugates Rtx-30.1699 and Rtx-30.2115 in a 96-hour CTG assay. Unconjugated rituximab was used as a reference compound.

FIG. 15. Results of cytotoxicity studies on (A) MEC-1-, (B) MEC-2-, (C) Raji-, (D) Nalm-6-, and (E) Ramos cell lines, respectively, using the rituximab amatoxin conjugates Rtx-30.1699 and Rtx-30.2115 in a 96-hour CTG assay in comparison to unconjugated α-amanitin.

FIG. 16. Results of cytotoxicity studies on (A) MEC-1-, (B) MEC-2-, (C) Raji-, (D) Nalm-6-, and (E) Ramos cell lines, respectively, using the obinutuzumab amatoxin conjugates Obi-30.1699 and Obi-30.2115 in a 96-hour CTG assay in comparison to unconjugated α-amanitin.

FIG. 17. Results of cytotoxicity studies using anti-CD20 amatoxin conjugates Rtx-30.2115 and Obi-30.2115 in vivo in a Scid mice Raji xenograft model system.

FIG. 18. Expression of CD20 in RS9737 and RS1316 cells. Data from RNA-seq analysis (whole transcriptome shotgun sequencing) are plotted as TPM (transcripts per million).

FIG. 19. Results of efficacy studies using anti-CD20 amatoxin conjugate Obi-30.1699 in vivo in patient-derived tumor xenograft models of Richter Syndrome with (A) RS9737 cells expressing low level of CD20 and (B) RS1316 cells expressing high levels of CD20.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, to prior art documents that disclose standard or routine methods. In that case, the incorporation by reference has mainly the purpose to provide sufficient enabling disclosure and avoid lengthy repetitions.

According to a first aspect of the present invention, the present invention relates to a conjugate comprising (i) a target binding moiety, (ii) at least one toxin, and (iii) optionally at least one linker connecting said target binding moiety with said at least one toxin, wherein said target binding moiety binds to CD20 and wherein said at least one toxin is an amatoxin.

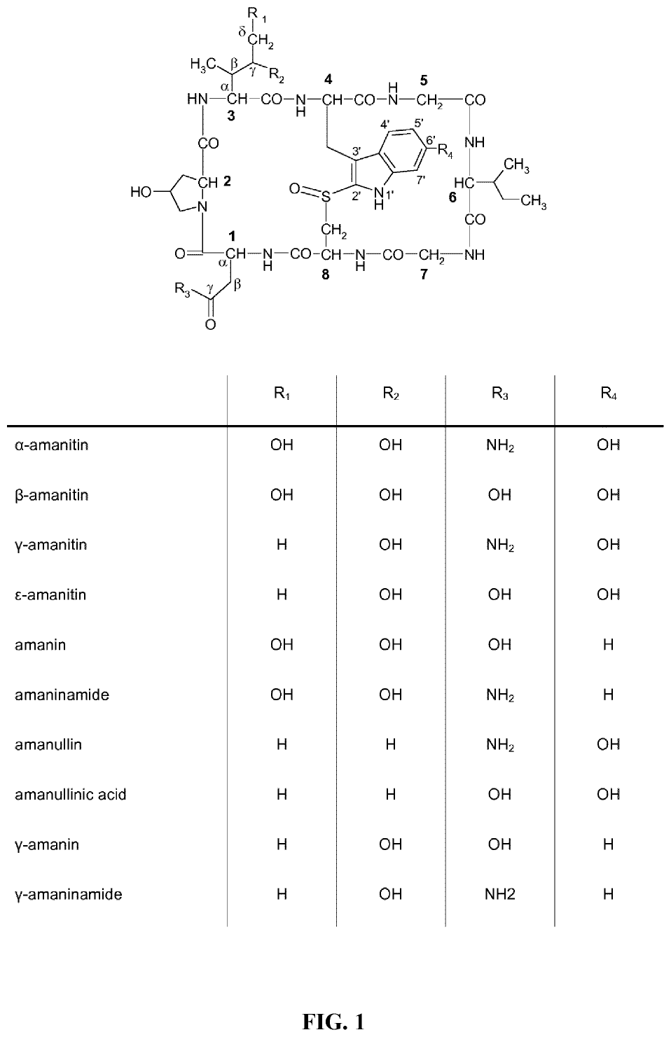

Amatoxins are cyclic peptides composed of 8 amino acids that are found in Amanita phalloides mushrooms (see FIG. 1). Amatoxins specifically inhibit the DNA-dependent RNA polymerase II of mammalian cells, and thereby also the transcription and protein biosynthesis of the affected cells. Inhibition of transcription in a cell causes stop of growth and proliferation. Though not covalently bound, the complex between amanitin and RNA-polymerase II is very tight (K_{D}=3 nM). Dissociation of amanitin from the enzyme is a very slow process, thus making recovery of an affected cell unlikely. When the inhibition of transcription lasts sufficiently long, the cell will undergo programmed cell death (apoptosis).

In the context of the present invention the term “amatoxin” includes all cyclic peptides composed of 8 amino acids as isolated from the genus Amanita and described in Wieland, T. and Faulstich H. (Wieland T, Faulstich H., CRC Crit Rev Biochem. 5 (1978) 185-260), further all chemical derivatives thereof; further all semisynthetic analogs thereof; further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin.

As used herein, a “derivative” of a compound refers to a species having a chemical structure that is similar to the compound, yet containing at least one chemical group not present in the compound and/or deficient of at least one chemical group that is present in the compound. The compound to which the derivative is compared is known as the “parent” compound. Typically, a “derivative” may be produced from the parent compound in one or more chemical reaction steps.

As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%, more preferably at least 40%, and more preferably at least 50%) of the activity of the parent compound. Thus, an “analogue of an amatoxin”, as it is used herein, refers to a compound that is structurally related to any one of α-amanitin, β-amanitin, γ-amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, and more preferably at least 50%) of the inhibitory activity against mammalian RNA polymerase II as compared to at least one of α-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of α-amanitin, β-amanitin, γ-amanitin, ε-amanitin, amanin, amaninamide, amanullin, or amanullinic acid. The inhibitory activity might be measured by determining the concentration at which 50% inhibition occurs (IC₅₀ value). The inhibitory activity against mammalian RNA polymerase II can be determined indirectly by measuring the inhibitory activity on cell proliferation.

A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes.

According to some embodiments of the present invention, the amatoxin can be selected from the group consisting of α-amanitin, β-amanitin, amanin, amaninamide and analogues, derivatives and salts thereof.

Functionally, amatoxins are defined as peptides or depsipeptides that inhibit mammalian RNA polymerase II. Preferred amatoxins are those with a functional group (e.g. a carboxylic group, an amino group, a hydroxy group, a thiol or a thiol-capturing group) that can be reacted with linker molecules or target-binding moieties as defined below.

In the context of the present invention, the term “amanitins” particularly refers to bicyclic structure that are based on an aspartic acid or asparagine residue in position 1, a proline residue, particularly a hydroxyproline residue in position 2, an isoleucine, hydroxyisoleucine or dihydroxyisoleucine in position 3, a tryptophan or hydroxytryptophan residue in position 4, glycine residues in positions 5 and 7, an isoleucine residue in position 6, and a cysteine residue in position 8, particularly a derivative of cysteine that is oxidized to a sulfoxide or sulfone derivative (for the numbering and representative examples of amanitins, see FIG. 1), and furthermore includes all chemical derivatives thereof; further all semisynthetic analogues thereof; further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase II.

The term “target-binding moiety”, as used herein, refers to any molecule or part of a molecule that can specifically bind to a target molecule or target epitope. Preferred target-binding moieties in the context of the present application are (i) antibodies or antigen-binding fragments thereof; (ii) antibody-like proteins; and (iii) nucleic acid aptamers. “Target-binding moieties” suitable for use in the present invention typically have a molecular mass of 40 000 Da (40 kDa) or more.

A “linker” in the context of the present application refers to a molecule that increases the distance between two components, e.g. to alleviate steric interference between the target binding moiety and the amatoxin, which may otherwise decrease the ability of the amatoxin to interact with RNA polymerase II. The linker may serve another purpose as it may facilitate the release of the amatoxin specifically in the cell being targeted by the target binding moiety. It is preferred that the linker and preferably the bond between the linker and the amatoxin on one side and the bond between the linker and the target binding moiety or antibody on the other side is stable under the physiological conditions outside the cell, e.g. the blood, while it can be cleaved inside the cell, in particular inside the target cell, e.g. cancer cell. To provide this selective stability, the linker may comprise functionalities that are preferably pH-sensitive or protease sensitive. Alternatively, the bond linking the linker to the target binding moiety may provide the selective stability. Preferably a linker has a length of at least 1, preferably of 1-30 atoms length (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 atoms), wherein one side of the linker has been reacted with the amatoxin and, the other side with a target-binding moiety. In the context of the present invention, a linker preferably is a C₁₋₃₀-alkyl, C₁₋₃₀-heteroalkyl, C₂₋₃₀-alkenyl, C₂₋₃₀-heteroalkenyl, C₂₋₃₀-alkynyl, C₂₋₃₀-heteroalkynyl, cycloalkyl, heterocycloalkyl, aryl, heteroaryl, aralkyl, or a heteroaralkyl group, optionally substituted. The linker may contain one or more structural elements such as amide, ester, ether, thioether, disulfide, hydrocarbon moieties and the like. The linker may also contain combinations of two or more of these structural elements. Each one of these structural elements may be present in the linker more than once, e.g. twice, three times, four times, five times, or six times. In some embodiments the linker may comprise a disulfide bond. It is understood that the linker has to be attached either in a single step or in two or more subsequent steps to the amatoxin and the target binding moiety. To that end the linker to be will carry two groups, preferably at a proximal and distal end, which can (i) form a covalent bond to a group, preferably an activated group on an amatoxin or the target binding-peptide or (ii) which is or can be activated to form a covalent bond with a group on an amatoxin. Accordingly, if the linker is present, it is preferred that chemical groups are at the distal and proximal end of the linker, which are the result of such a coupling reaction, e.g. an ester, an ether, a urethane, a peptide bond etc. The presence of a “linker” is optional, i.e. the toxin may be directly linked to a residue of the target-binding moiety in some embodiments of the target-binding moiety toxin conjugate.

The present invention further relates to a conjugate comprising a target binding moiety binding to CD20, at least one amatoxin and optionally a linker, wherein said target binding moiety is selected from the group consisting of
(i) an antibody, preferably a monoclonal antibody,
(ii) an antigen-binding fragment thereof, preferably a variable domain (Fv), a Fab fragment or an F(ab)₂ fragment,
(iii) an antigen-binding derivative thereof, preferably a single-chain Fv (scFv), and
(iv) an antibody-like protein, each binding to CD20, respectively.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or V_{H}) and a heavy chain constant region (abbreviated as CH or C_{H}). The heavy chain constant region is comprised of three domains, namely CH1, CH2 and CH3. Each light chain contains a light chain variable region (abbreviated as VL or V_{L}) and a light chain constant region (abbreviated as CL or C_{L}). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

The term “antigen binding portion” of the (monoclonal) antibody refers to one or more fragments of an antibody which retain the ability to specifically bind to the CD20 antigen in its native form. Said CD20 antigen can be a mammalian, non-primate, primate, and in particular a human CD20 antigen. “CD20” hereby refers to a protein comprising of or consisting of the amino acid sequence according to SEQ ID NO: 6, or a sequence that is at least 90%, 92.5%, 95%, or at least 97% identical to SEQ ID NO: 6. Examples of antigen binding portions of the antibody include a Fab fragment, a monovalent fragment consisting of the VL, VH, CL and CH1 domains, an F(ab′)₂ fragment, a bivalent fragment comprising two Fab fragments linked by a disulfid bridge at the hinge region, an Fd fragment consisting of the VH and CH1 domain, an Fv fragment consisting of the VL and VH domains of a single arm of an antibody, and a dAb fragment which consists of a VH domain and an isolated complementarity determining region (CDR).

Sequence identity according to the invention may e.g. be determined over the whole length of each of the sequences being compared to a respective reference sequence (so-called “global alignment”), that is particularly suitable for sequences of the same or similar length, or over shorter, defined lengths (so-called “local alignment”), that is more suitable for sequences of unequal length. In the above context, an amino acid sequence having a “sequence identity” of at least, for example, 95% to a query amino acid sequence, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted. Methods for comparing the identity and homology of two or more sequences are well known in the art. The percentage to which two sequences are identical can for example be determined by using a mathematical algorithm. A preferred, but not limiting, example of a mathematical algorithm which can be used is the algorithm of Karlin et al. (1993), PNAS USA, 90:5873-5877. Such an algorithm is integrated in the BLAST family of programs (see also Altschul et al., 1990, J. Mol. Biol. 215, 403-410 or Altschul et al. (1997), Nucleic Acids Res, 25:3389-3402), accessible through the home page of the NCBI at world wide web site ncbi.nlm.nih.gov) and FASTA (Pearson (1990), Methods Enzymol. 83, 63-98; Pearson and Lipman (1988), Proc. Natl. Acad. Sci. U.S.A 85, 2444-2448.). Sequences which are identical to other sequences to a certain extent can be identified by these programs. Furthermore, programs available in the Wisconsin Sequence Analysis Package (Devereux et al, 1984, Nucleic Acids Res., 387-395; Womble Methods Mol Biol. 2000; 132:3-22), for example the programs BESTFIT and GAP, may be used to determine the % identity between two polypeptide sequences.

The antibody, or antibody fragment or antibody derivative thereof, according to the present invention can be a monoclonal antibody. The antibody can be of the IgA, IgD, IgE, IgG or IgM isotype.

As used herein, the term “monoclonal antibody (mAb)” shall refer to an antibody composition having a homogenous antibody population, i.e., a homogeneous population consisting of a whole immunoglobulin, or a fragment or derivative thereof. Particularly preferred, such antibody is selected from the group consisting of IgG, IgD, IgE, IgA and/or IgM, or a fragment or derivative thereof. Monoclonal antibodies may e.g. be made by the hybridoma method first described by Kohler and Milstein, Nature 256:495 (1975); Eur. J. Immunol. 6:511 (1976), by recombinant DNA techniques, or may also be isolated from phage antibody libraries.

As used herein, the term “fragment”, or “antibody fragment” shall refer to fragments of such antibody retaining target binding capacities, e.g., a CDR (complementarity determining region), a hypervariable region, a variable domain (Fv), an IgG heavy chain (consisting of VH, CH1, hinge, CH2 and CH3 regions), an IgG light chain (consisting of VL and CL regions), and/or a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs. All these items are explained below.

Other antibody derivatives known to the skilled person are Diabodies, Camelid Antibodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, other scaffold protein formats comprising CDRs, and antibody conjugates (e.g., antibody, or fragments or derivatives thereof, linked to a drug, a toxin, a cytokine, an aptamer, a nucleic acid such as a desoxyribonucleic acid (DNA) or ribonucleic acid (RNA), a therapeutic polypeptide, a radioisotope or a label). Said scaffold protein formats may comprise, for example, antibody-like proteins such as ankyrin and affilin proteins and others.

As used herein, the term “antibody-like protein” refers to a protein that has been engineered (e.g. by mutagenesis of Ig loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin repeat proteins (Binz et al., 2005). Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins.

As used herein, the term “Fab” relates to an IgG fragment comprising the antigen binding region, said fragment being composed of one constant and one variable domain from each heavy and light chain of the antibody.

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually comprising serine(S) and/or glycine (G) residues. This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like.

IgG, scFv, Fab and/or F(ab)₂ are antibody formats which are well known to the skilled person. Related enabling techniques are available from respective textbooks.

According to preferred embodiments of the present invention, said antibody, or antigen-binding fragment thereof or antigen-binding derivative thereof, is a murine, a chimeric, a humanized or a human antibody, or antigen-binding fragment or antigen-binding derivative thereof, respectively.

Monoclonal antibodies (mAb) derived from mouse may cause unwanted immunological side-effects due to the fact that they contain a protein from another species which may elicit antibodies. In order to overcome this problem, antibody humanization and maturation methods have been designed to generate antibody molecules with minimal immunogenicity when applied to humans, while ideally still retaining specificity and affinity of the nonhuman parental antibody (for review see Almagro and Fransson 2008). Using these methods, e.g., the framework regions of a mouse mAb are replaced by corresponding human framework regions (so-called CDR grafting). WO200907861 discloses the generation of humanized forms of mouse antibodies by linking the CDR regions of non-human antibodies to human constant regions by recombinant DNA technology. U.S. Pat. No. 6,548,640 by Medical Research Council describes CDR grafting techniques, and U.S. Pat. No. 5,859,205 by Celltech describes the production of humanised antibodies.

As used herein, the term “humanized antibody” relates to an antibody, a fragement or a derivative thereof, in which at least a portion of the constant regions and/or the framework regions, and optionally a portion of CDR regions, of the antibody is derived from or adjusted to human immunoglobulin sequences.

The antibodies, the antibody fragments or antibody derivatives thereof, disclosed herein can comprise humanized sequences, in particular of the preferred VH- and VL-based antigen-binding region which maintain appropriate ligand affinity. The amino acid sequence modifications to obtain said humanized sequences may occur in the CDR regions and/or in the framework regions of the original antibody and/or in antibody constant region sequences.

Said antibody, or antibody fragment or antibody derivative thereof, can be glycosylated. The glycan can be an N-linked oligosaccharide chain at asparagin 297 of the heavy chain.

The antibodies or fragments or derivatives of the present invention may be produced by transfection of a host cell with an expression vector comprising the coding sequence for the antibody according to the invention. The expression vector or recombinant plasmid is produced by placing the coding antibody sequences under control of suitable regulatory genetic elements, including promoter and enhancer sequences like, e.g., a CMV promoter. Heavy and light chain sequences might be expressed from individual expression vectors which are co-transfected, or from dual expression vectors. Said transfection may be a transient transfection or a stabile transfection. The transfected cells are subsequently cultivated to produce the transfected antibody construct. When stable transfection is performed, then stable clones secreting antibodies with properly associated heavy and light chains are selected by screening with an appropriate assay, such as, e.g., ELISA, subcloned, and propagated for future production.

According to preferred embodiments of the present invention, said antibody, or antigen-binding fragment thereof, or antigen-binding derivative thereof, respectively, is selected from the group consisting of rituximab, obinutuzumab, ibritumomab, tositumomab, ofatumumab, ocrelizumab, and ublituximab. The international nonproprietary names (INNs) as used herein are meant to also encompass all biosimilar antibodies having the same, or substantially the same, amino acid sequences and/or glycosylation pattern as the originator antibody as disclosed above, in accordance with 42 USC § 262 subsection (i) or equivalent regulations in other jurisdictions.

According to some embodiments of the present invention, said antibody as disclosed herein is genetically engineered to comprise a heavy chain 118Cys, a heavy chain 239Cys, or heavy chain 265Cys according to the EU numbering system (see e.g. Proc. Natl. Acad. Sci. USA 1969, 63, 78-85), preferably a heavy chain 265Cys according to the EU numbering system, and wherein said linker, if present, or said amatoxin is connected to said antibody via said heavy chain 118Cys, or said heavy chain 239Cys, or heavy chain 265Cys residue, respectively. For example, WO2006/034488 A2 discloses corresponding methods for manufacturing of cysteine-engineered antibodies.

According to one embodiment of the present invention, said antibody is rituximab that has been genetically engineered to comprise a heavy chain 265Cys according to the EU numbering system.

As used herein, the term “genetically engineered” or “genetic engineering” relates to the modification of the amino acid sequence or part thereof of a given or natural polypeptide or protein in the sense of nucleotide and/or amino acid substitution, insertion, deletion or reversion, or any combinations thereof, by gene technological methods.

As used herein, the term “amino acid substitution” relates to modifications of the amino acid sequence of the protein, wherein one or more amino acids are replaced with the same number of different amino acids, producing a protein which contains a different amino acid sequence than the original protein. A conservative amino acid substitution is understood to relate to a substitution which due to similar size, charge, polarity and/or conformation does not significantly affect the structure and function of the protein. Groups of conservative amino acids in that sense represent, e.g., the non-polar amino acids Gly, Ala, Val, Ile and Leu; the aromatic amino acids Phe, Trp and Tyr; the positively charged amino acids Lys, Arg and His; and the negatively charged amino acids Asp and Glu.

According to a preferred embodiment of the present invention, said linker, if present, or said amatoxin, or said amatoxin coupled to said linker is connected to said antibody via any of the naturally occurring Cys residues of said antibody, preferably via a disulfide linkage. The term “naturally occurring Cys residues” as used herein refers to cysteine residues that are present in native antibodies, such as rituximab, and which form intrachain disulfide bonds in the light and heavy chain of an antibody, or which form interchain disulfide bonds between heavy and light chains and/or between the heavy chains of the antibody, such as those disulfide bonds in the hinge region of IgG immunoglobulins. Preferred naturally occurring Cys residues for connecting or coupling said linker, if present, or said amatoxin or said amatoxin coupled to said linker as disclosed herein are the cysteine residues which form the interchain disulfide bonds which link both heavy chains in the hinge region of native IgG immunoglobulins. Accordingly, the amatoxin, the linker, or the amatoxin coupled to a linker as disclosed herein are e.g. coupled to the antibody via the cysteine residues that form the interchain disulfide bonds. The coupling to cysteines residues which contribute to the interchain disulfide bond in the native antibody may e.g. be done according to methods as disclosed in mAbs 6:1, 46-53 (2014), or Clinical Cancer Research Vol. 10, 7063-7070 Oct. 15, 2004.

According to a particularly preferred embodiment of the present invention, said antibody is rituximab.

In a preferred embodiment, the antibody, or antibody fragment or antibody derivative thereof, of said conjugate binds to an extracellular domain of the CD20 molecule.

In a preferred embodiment, the invention relates to a conjugate comprising an antibody, or antibody fragment or antibody derivative thereof as described above, wherein the same binds to the extracellular domain of CD20.

Furthermore, the conjugate according to the present invention can have a cytotoxic activity of an IC₅₀ better than 10×10⁻⁹ M, 9×10⁻⁹ M, 8×10⁻⁹ M, 7×10⁻⁹ M, 6×10⁻⁹ M, 5×10⁻⁹ M, 4×10⁻⁹ M, 3×10⁻⁹ M, 2×10⁻⁹ M, preferably better than 10×10⁻¹⁰ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, and more preferably better than 10×10⁻¹¹ M, 9×10⁻¹¹ M, 8×10⁻¹¹ M, 7×10⁻¹¹ M, 6×10⁻¹¹ M, 5×10⁻¹¹ M, 4×10⁻¹¹ M, 3×10⁻¹¹ M, 2×10⁻¹¹ M, or 1×10⁻¹¹ M.

In a preferred embodiment of the present invention, said conjugate as described comprises an amatoxin comprising (i) an amino acid 4 with a 6′-deoxy position and (ii) an amino acid 8 with an S-deoxy position.

According to preferred embodiments of the present invention, said conjugate as described comprises a linker, wherein said linker is a non-cleavable or a cleavable linker.

Said cleavable linker can be selected from the group consisting of an enzymatically cleavable linker, preferably a protease-cleavable linker, and a chemically cleavable linker, preferably a linker comprising a disulfide bridge.

A “cleavable linker” is understood as comprising at least one cleavage site. As used herein, the term “cleavage site” shall refer to a moiety that is susceptible to specific cleavage at a defined position under particular conditions. Said conditions are, e.g., specific enzymes or a reductive environment in specific body or cell compartments.

According to embodiments of the invention, the cleavage site is an enzymatically cleavable moiety comprising two or more amino acids. Preferably, said enzymatically cleavable moiety comprises a valine-alanine (Val-Ala), valine-citrulline (Val-Cit), valine-lysine (Val-Lys), valine-arginine (Val-Arg) dipeptide, a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside.

According to some embodiments, said cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands co-ordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme's active site. Serine proteases fall into two broad categories based on their structure: chymotrypsin-like (trypsin-like) or subtilisin-like.

Threonine proteases are a family of proteolytic enzymes harbouring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In particular embodiments of the present invention, the cleavable site is cleavable by at least one agent selected from the group consisting of Cathepsin A or B, matrix metalloproteinases (MMPs), elastases, β-glucuronidase and β-galactosidase.

In particular embodiments of the present invention, the cleavage site is a disulfide bond and specific cleavage is conducted by a reductive environment, e.g., an intracellular reductive environment, such as, e.g., acidic pH conditions.

According to preferred embodiments of the present invention, in said conjugate as described, said linker, if present, or said target binding moiety is connected to said amatoxin via (i) the Y C-atom of amatoxin amino acid 1, or (ii) the 8 C-atom of amatoxin amino acid 3, or (iii) the 6′-C-atom of amatoxin amino acid 4.

According to particularly preferred embodiments of the present invention, said conjugate is comprising any of the following compounds of formulas (I) to (XII), respectively, as linker-amatoxin moieties:

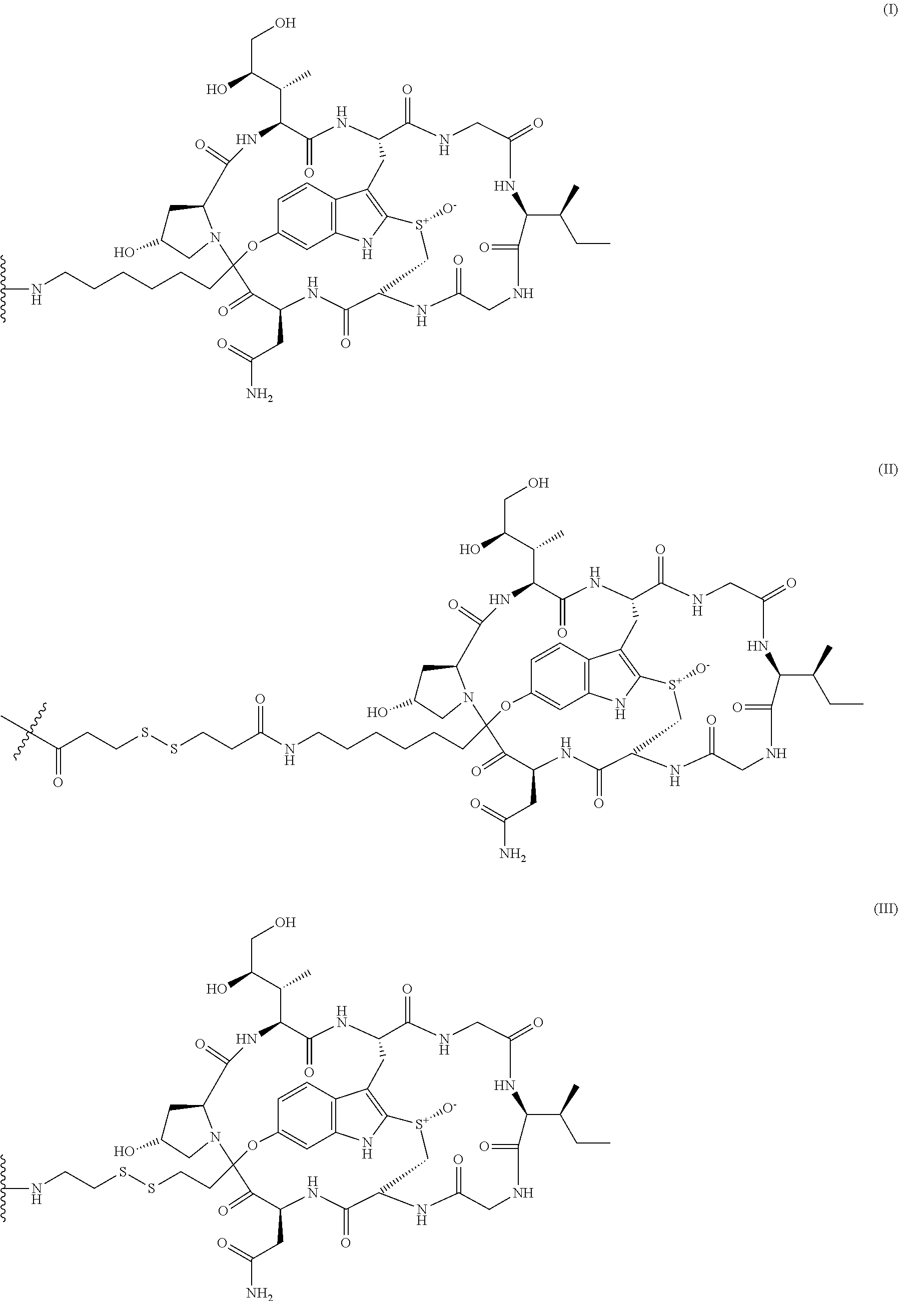

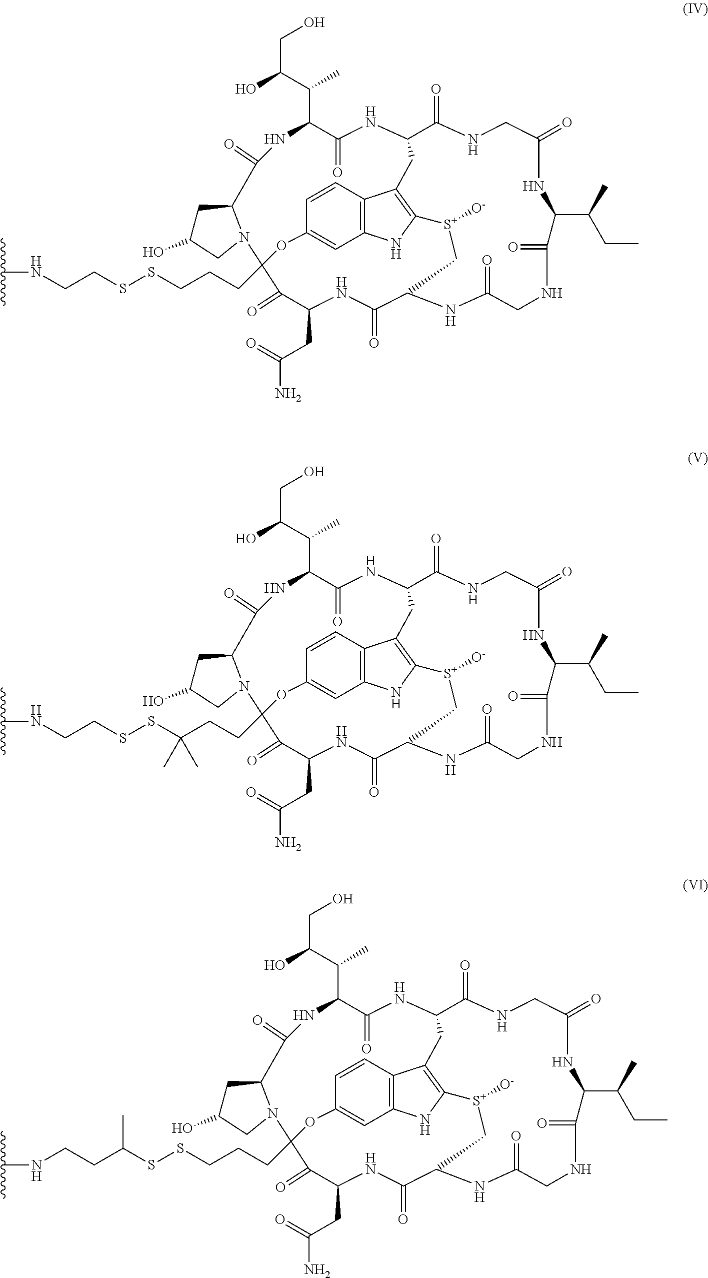

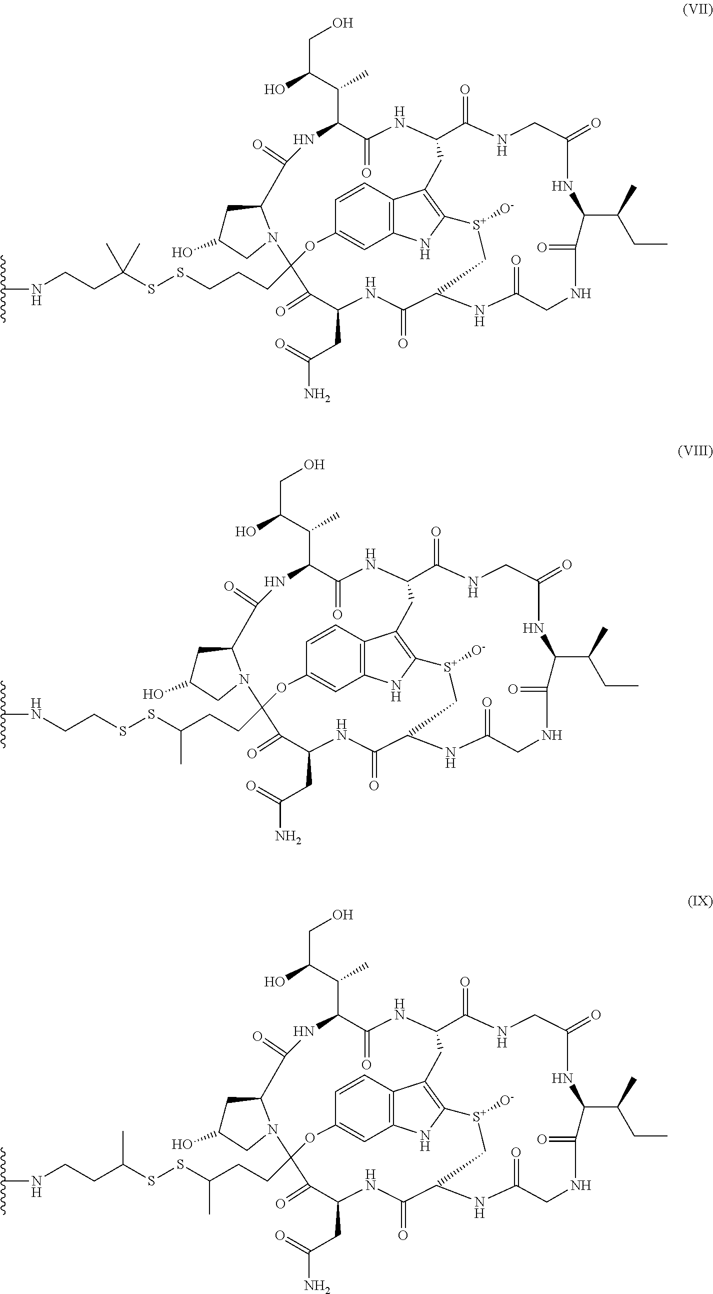

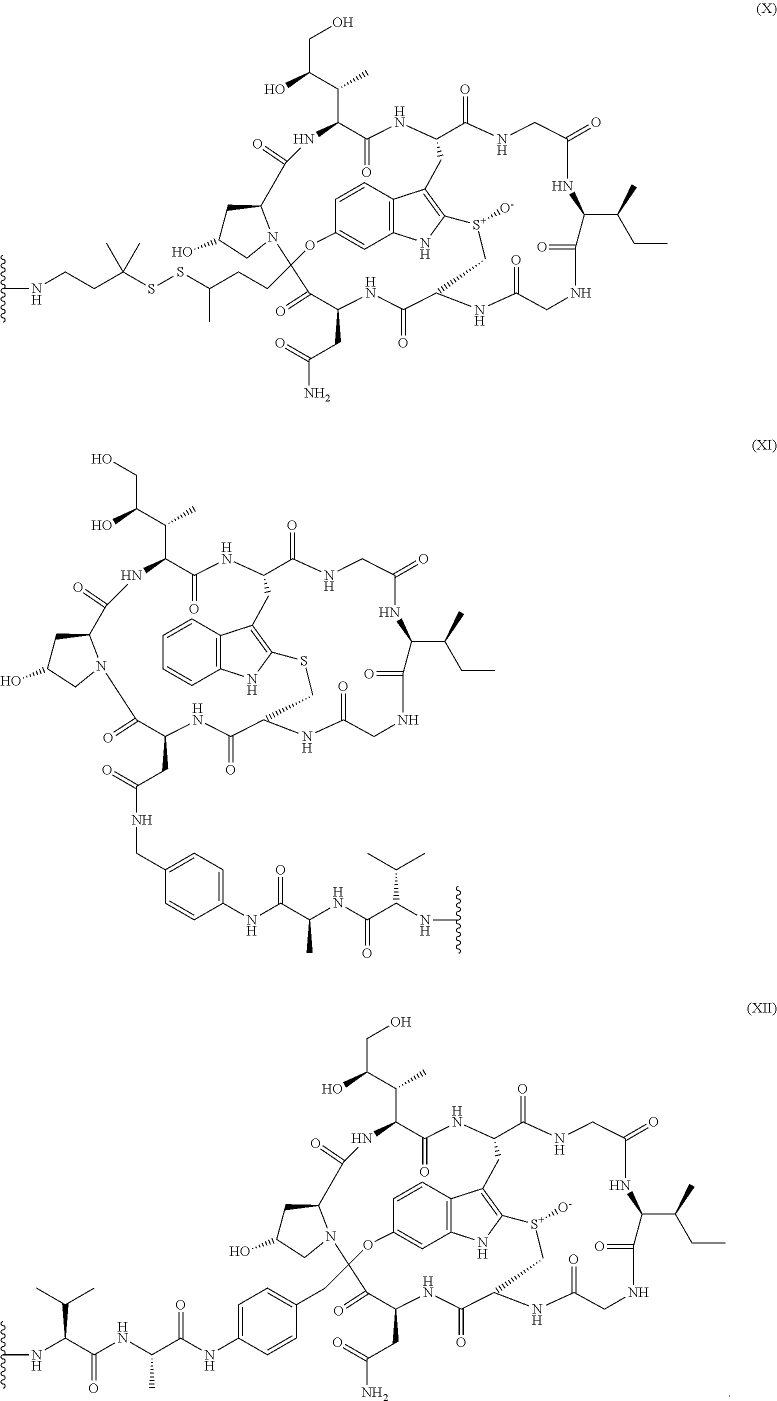

Furthermore, according to particularly preferred embodiments of the present invention, said conjugate is comprising an antibody as target binding moiety conjugated to amatoxin linker moieties according to any one of formula XIII to XXII

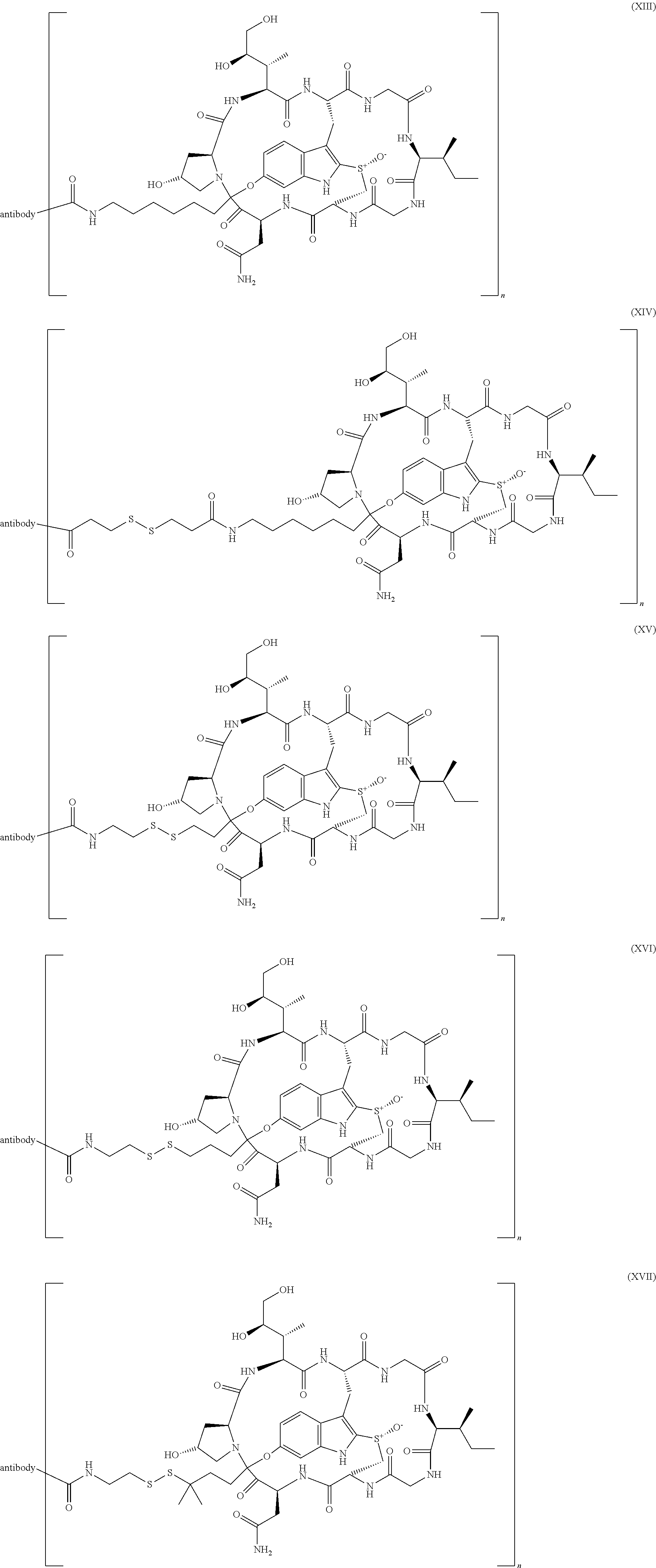

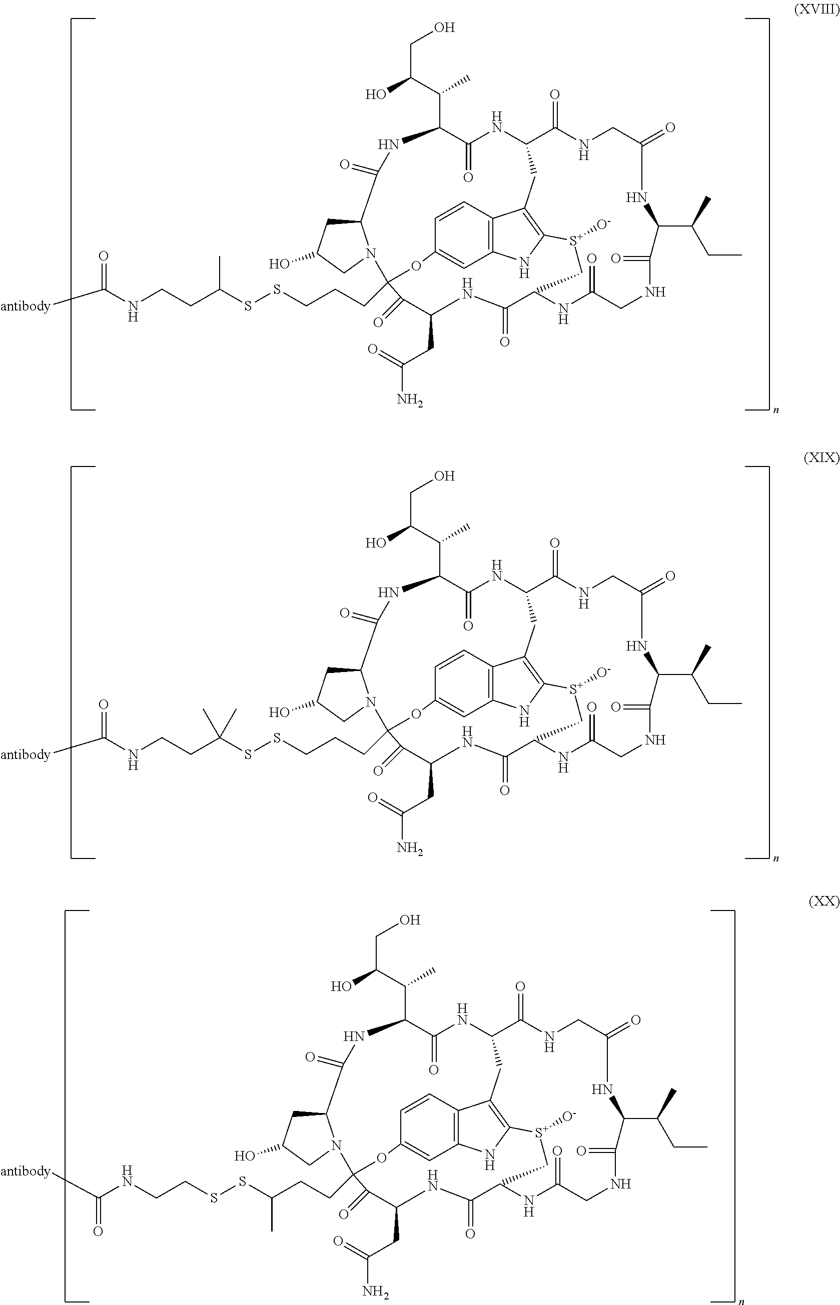

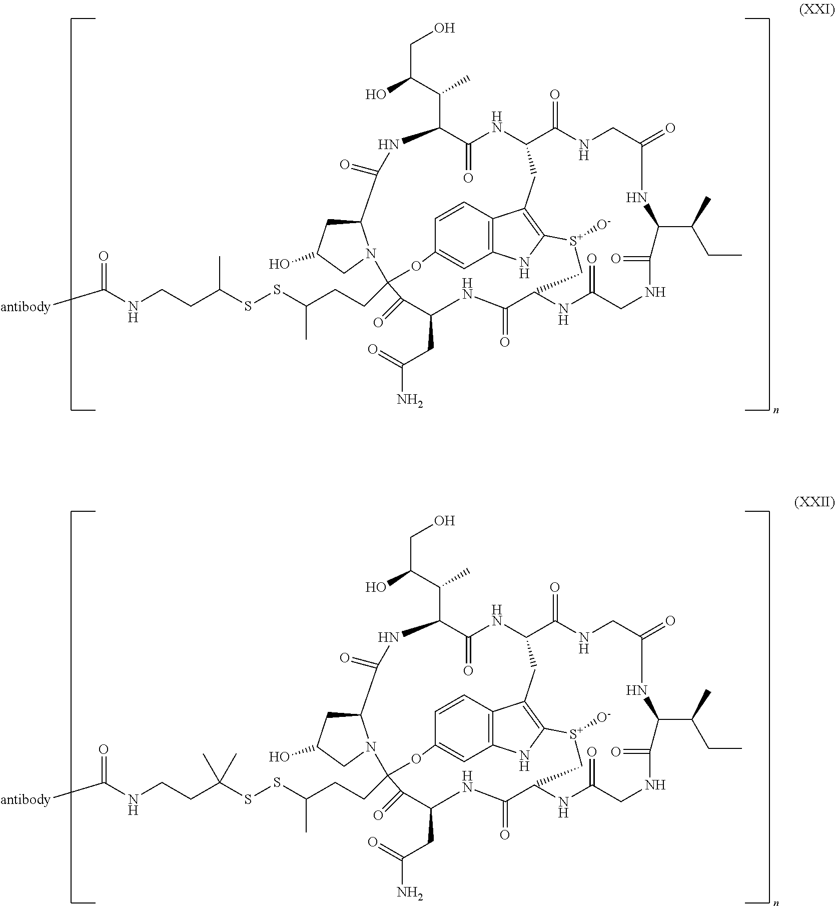

wherein the amatoxin linker moieties are coupled to ε-amino groups of naturally occurring lysine residues of said antibody, and wherein n is preferably from 1 to 7.

Furthermore, according to particularly preferred embodiments of the present invention, said conjugate is comprising an antibody as target binding moiety conjugated to amatoxin linker moieties according to any one of formula XXIII and XXIV

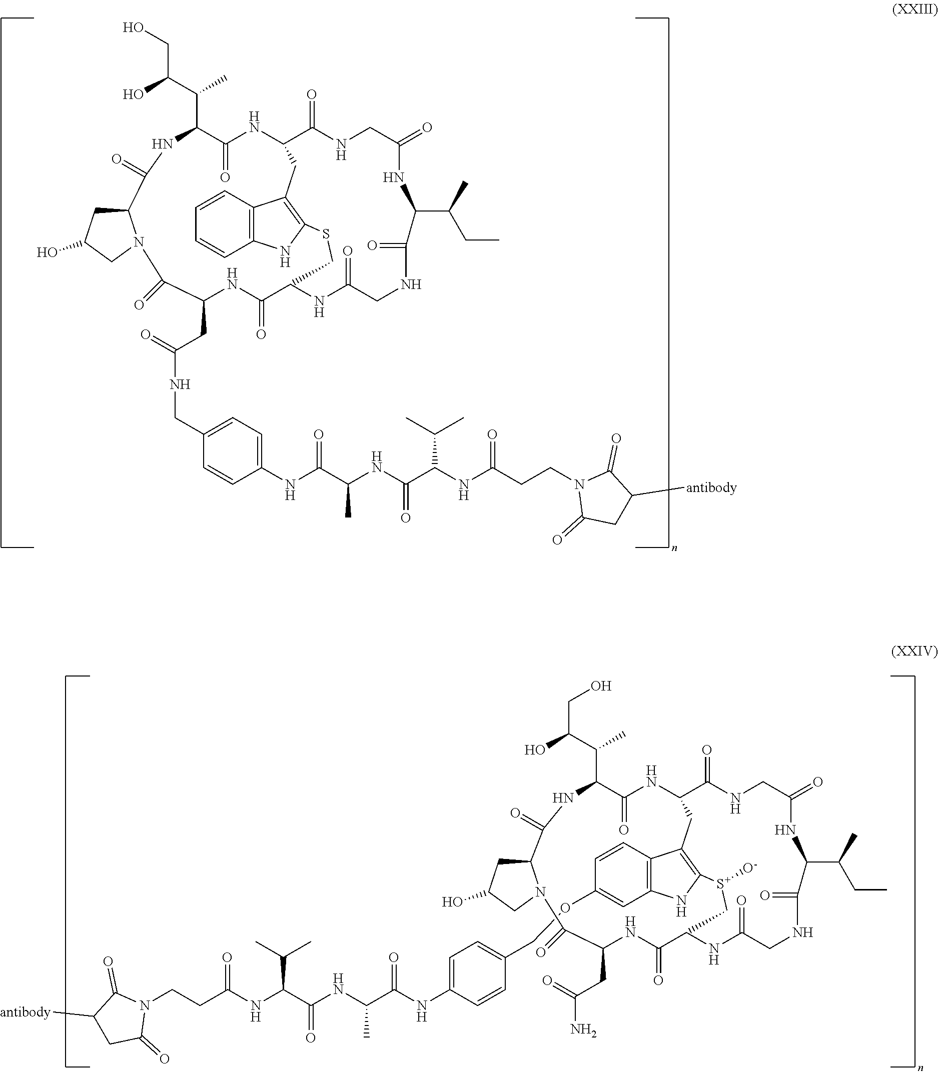

wherein the amatoxin linker moieties are coupled to the thiol groups of cysteine residues of the antibody, and wherein n is preferably from 1 to 7.

According to even more particularly preferred embodiments of the present invention, said conjugate is selected from the group consisting of
(i) a conjugate comprising the antibody rituximab as target binding moiety conjugated to at least one amatoxin-linker moiety of formula (XI) via thioether linkage to at least one naturally occurring Cys residue of rituximab, e.g. to at least one Cys residue that contributes to the interchain disulfide bonds of rituximab, according to formula XXV

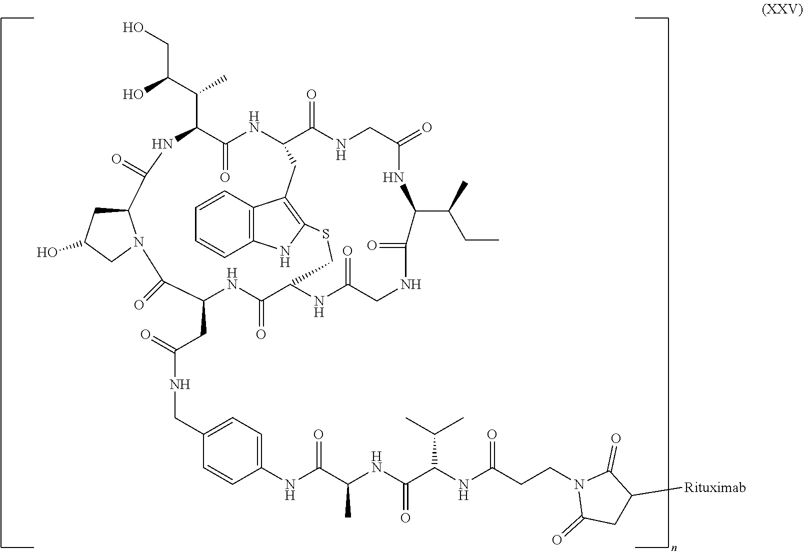

(ii) a conjugate comprising the antibody rituximab genetically engineered to comprise a heavy chain 265Cys according to the EU numbering system as target binding moiety conjugated to an amatoxin linker moiety of formula (XI) via thioether linkage to said heavy chain 265Cys residue of said genetically engineered rituximab, according to formula XXVI,

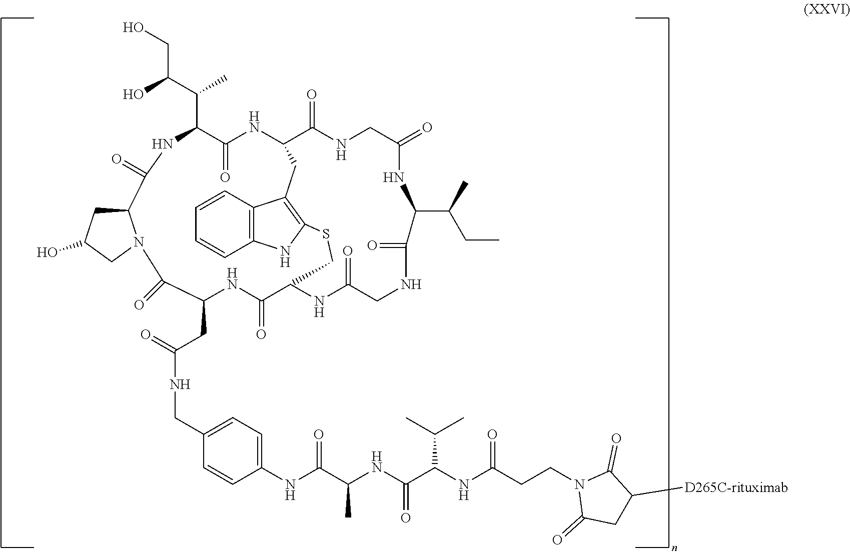

(iii) a conjugate comprising the antibody rituximab as target binding moiety conjugated to at least one amatoxin-linker moiety of formula (XII) via thioether linkage, or to at least one naturally occurring Cys residue of rituximab, e.g. to at least one Cys residue that contributes to the interchain disulfide bonds of rituximab, according to formula XXVII

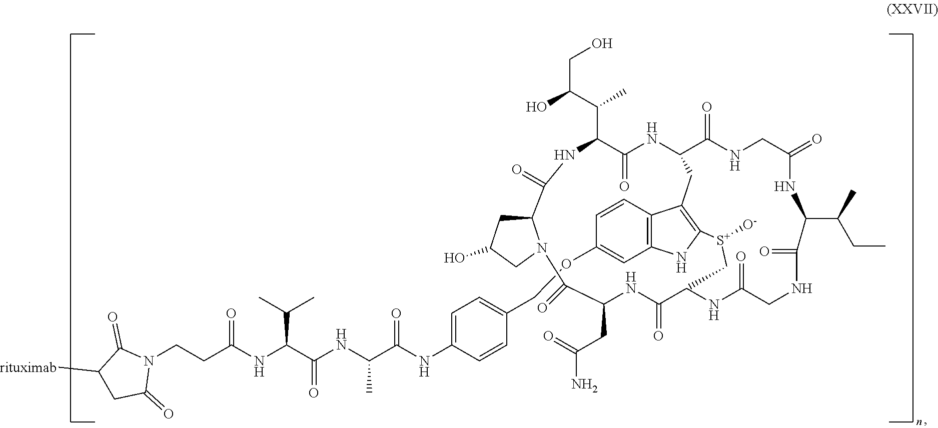

(iv) a conjugate comprising the antibody rituximab genetically engineered to comprise a heavy chain 265Cys according to the EU numbering system as target binding moiety conjugated to an amatoxin linker moiety of formula (XII) via thioether linkage to said heavy chain 265Cys residue of said genetically engineered rituximab, according to formula XXVIII

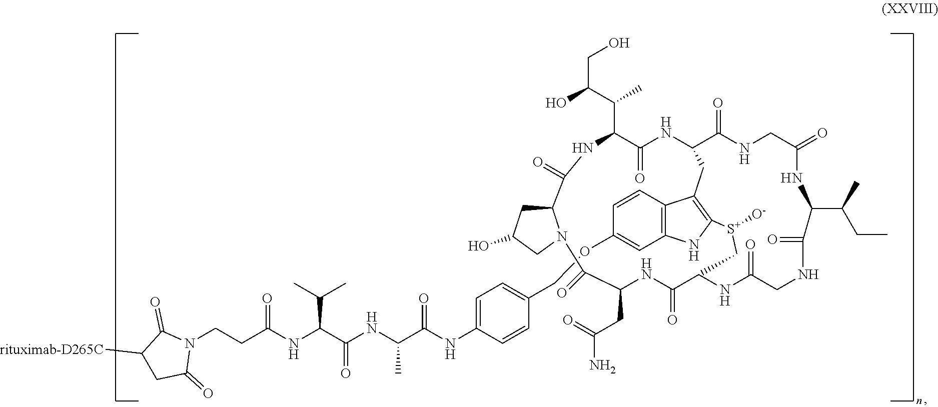

and wherein n is 1 to 7 for (i), (iii), and n is 1 to 2 for (ii), (iv).

According to another aspect of the present invention, the present invention relates to a pharmaceutical composition comprising said conjugate as described.

Said pharmaceutical composition may comprise one or more pharmaceutically acceptable buffers, surfactants, diluents, carriers, excipients, fillers, binders, lubricants, glidants, disintegrants, adsorbents, and/or preservatives.

In aqueous form, said pharmaceutical formulation may be ready for administration, while in lyophilised form said formulation can be transferred into liquid form prior to administration, e.g., by addition of water for injection which may or may not comprise a preservative such as for example, but not limited to, benzyl alcohol, antioxidants like vitamin A, vitamin E, vitamin C, retinyl palmitate, and selenium, the amino acids cysteine and methionine, citric acid and sodium citrate, synthetic preservatives like the parabens methyl paraben and propyl paraben.

Said pharmaceutical formulation may further comprise one or more stabilizer, which may be, e.g., an amino acid, a sugar polyol, a disaccharide, and/or a polysaccharide. Said pharmaceutical formulation may further comprise one or more surfactant, one or more isotonizing agents, and/or one or more metal ion chelator, and/or one or more preservative.

The pharmaceutical formulation as described herein can be suitable for at least intravenous, intramuscular, or subcutaneous administration. Alternatively, said conjugate according to the present invention may be provided in a depot formulation which allows the sustained release of the biologically active agent over a certain period of time.

In still another aspect of the present invention, a primary packaging, such as a prefilled syringe or pen, a vial, or an infusion bag is provided, which comprises said formulation according to the previous aspect of the invention.

The prefilled syringe or pen may contain the formulation either in lyophilised form (which has then to be solubilised, e.g., with water for injection, prior to administration), or in aqueous form. Said syringe or pen is often a disposable article for single use only, and may have a volume between 0.1 and 20 ml. However, the syringe or pen may also be a multi-use or multi-dose syringe or pen.

Said vial may also contain the formulation in lyophilised form or in aqueous form and may serve as a single or multiple use device. As a multiple use device, said vial can have a bigger volume. Said infusion bag usually contains the formulation in aqueous form and may have a volume between 20 and 5000 ml.

According to another aspect of the present invention, the present invention relates to said conjugate or pharmaceutical composition as described for use in the treatment of B lymphocyte-associated malignancies or B cell-mediated autoimmune diseases, in particular for use in the treatment of non-Hodgkin's lymphoma, follicular lymphoma, diffuse large B cell non-Hodgkin's lymphoma, chronic lymphocytic leukaemia, Richter syndrome, rheumatoid arthritis, granulomatosis with polyangiitis and microscopic polyangiitis and pemphigus vulgaris.

The invention relates to the use of said conjugate or pharmaceutical composition for the treatment of B lymphocyte-associated malignancies or B cell-mediated autoimmune diseases, in particular for treatment of non-Hodgkin's lymphoma, follicular lymphoma, diffuse large B cell non-Hodgkin's lymphoma, chronic lymphocytic leukaemia, Richter syndrome, rheumatoid arthritis, granulomatosis with polyangiitis and microscopic polyangiitis and pemphigus vulgaris.

The Richter syndrome is defined as the transformation of chronic lymphocytic leukemia (CLL)/small lymphocytic lymphoma into an aggressive lymphoma, most commonly a diffuse large B-cell lymphoma (DLBCL). Occurring in approximately 2%-10% of patients with CLL, Richter syndrome is highly aggressive, often refractory to treatment, with a poor outcome of approximately 8-14 months. Approximately 80% of cases are clonally related to the underlying CLL, while the remaining 20% of patients have a clonally unrelated DLBCL and have a better prognosis similar to that of de novo DLBCL (Vaisitti et al 2018). A combination of germline genetic characteristics, clinical features, biologic and somatic genetic characteristics of CLL B cells, and certain CLL therapies are associated with higher risk of Richter syndrome.

The invention relates also to a method of treating a patient suffering from a B lymphocyte-associated malignancy or B cell-mediated autoimmune disease, comprising administering an effective amount of said conjugate or pharmaceutical composition to the patient. For example, the method of treating a patient suffering from a B lymphocyte-associated malignancy or B cell-mediated autoimmune disease as disclosed herein, comprises administering to said patient from about 0.1 mg/kg body weight to about 25 mg/kg body weight of said conjugate or pharmaceutical composition to said patient, whereby said conjugate or pharmaceutical composition is administered at least once to said patient. A preferred route of administration of said conjugate or pharmaceutical composition may e.g. comprise intravenous (i.v.) administration, or subcutaneous (s.c.) administration in a therapeutically effective amount.

TABLE 1
Sequences
Amino acid sequences which are preferred
antibody sequences of the invention

| SEQ ID No. | Sequence | Description |
|---|---|---|
| SEQ ID No. 1 | QIVLSQSPAILSASPGEKVTMTCRASSSVS | Rituximab light chain |
| | YIHWFQQKPGSSPKPWIYATSNLASGVPVR | |
| | FSGSGSGTSYSLTISRVEAEDAATYYCQQW | |
| | TSNPPTFGGGTKLEIKRTVAAPSVFIFPPS | |
| | DEQLKSGTASVVCLLNNFYPREAKVQWKVD | |
| | NALQSGNSQESVTEQDSKDSTYSLSSTLTL | |
| | SKADYEKHKVYACEVTHQGLSSPVTKSFNR | |
| | GEC | |
| SEQ ID No. 2 | QVQLQQPGAELVKPGASVKMSCKASGYTFT | Rituximab heavy chain |
| | SYNMHWVKQTPGRGLEWIGAIYPGNGDTSY | |
| | NQKFKGKATLTADKSSSTAYMQLSSLTSED | |
| | SAVYYCARSTYYGGDWYFNVWGAGTTVTVS | |
| | AASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKAEPKSCDKTHTCPPCPAPELLG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALPAPIEKTISKAKGQPREPQVYTLPPSRD | |
| | ELTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| SEQ ID No. 3 | QVQLQQPGAELVKPGASVKMSCKASGYTFT | Rituximab 265Cys heavy |
| | SYNMHWVKQTPGRGLEWIGAIYPGNGDTSY | chain |
| | NQKFKGKATLTADKSSSTAYMQLSSLTSED | |
| | SAVYYCARSTYYGGDWYFNVWGAGTTVTVS | |
| | AASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKAEPKSCDKTHTCPPCPAPELLG | |
| | GPSVFLFPPKPKDTLMISRTPEVTCVVVCV | |
| | SHEDPEVKFNWYVDGVEVHNAKTKPREEQY | |
| | NSTYRVVSVLTVLHQDWLNGKEYKCKVSNK | |
| | ALPAPIEKTISKAKGQPREPQVYTLPPSRD | |
| | ELTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| SEQ ID No. 4 | QVQLQQPGAELVKPGASVKMSCKASGYTFT | Rituximab 118Cys heavy |
| | SYNMHWVKQTPGRGLEWIGAIYPGNGDTSY | chain |
| | NQKFKGKATLTADKSSSTAYMQLSSLTSED | |
| | SAVYYCARSTYYGGDWYFNVWGAGTTVTVS | |
| | ACSTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKAEPKSCDKTHTCPPCPAPELLG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALPAPIEKTISKAKGQPREPQVYTLPPSRD | |
| | ELTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| SEQ ID No. 5 | QVQLQQPGAELVKPGASVKMSCKASGYTFT | Rituximab 239Cys heavy |
| | SYNMHWVKQTPGRGLEWIGAIYPGNGDTSY | chain |
| | NQKFKGKATLTADKSSSTAYMQLSSLTSED | |
| | SAVYYCARSTYYGGDWYFNVWGAGTTVTVS | |
| | AASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKAEPKSCDKTHTCPPCPAPELLG | |
| | GPCVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALPAPIEKTISKAKGQPREPQVYTLPPSRD | |
| | ELTKNQVSLTCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| SEQ ID NO: 6 | MTTPRNSVNGTFPAEPMKGPIAMQSGPKPLF | human CD20 |
| | RRMSSLVGPTQSFFMRESKTLGAVQIMNGLF | |
| | HIALGGLLMIPAGIYAPICVTVWYPLWGGIM | |
| | YIISGSLLAATEKNSRKCLVKGKMIMNSLSL | |
| | FAAISGMILSIMDILNIKISHFLKMESLNFI | |
| | RAHTPYINIYNCEPANPSEKNSPSTQYCYSI | |
| | QSLFLGILSVMLIFAFFQELVIAGIVENEWK | |
| | RTCSRPKSNIVLLSAEEKKEQTIEIKEEVVG | |
| | LTETSSQPKNEEDIEIIPIQEEEEEETETNF | |
| | PEPPQDQESSPIENDSSP | |

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Example 1: Binding of Rituximab to Lymphoma Cell Lines

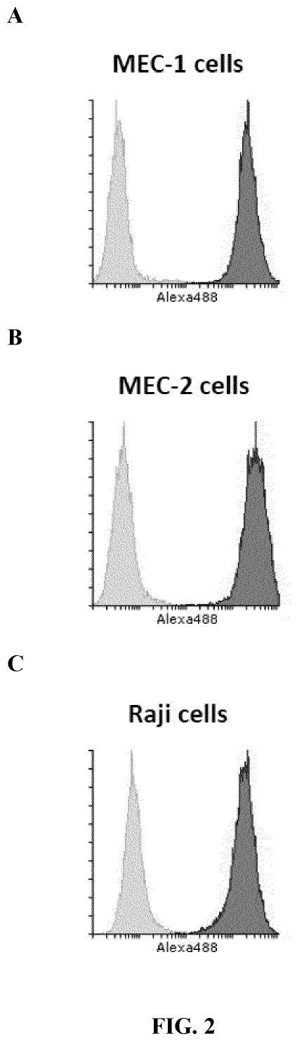

Binding of the anti-CD20 monoclonal antibody rituximab to the CD20-positive human chronic B-cell leukemia cell lines MEC-1 and MEC-2 as well as to the human Burkitt lymphoma cell line Raji were tested by FACS analysis. Rituximab was shown to bind strongly to the CD20-positive MEC-1, MEC-2 and Raji cell lines (FIG. 2).

### Example 2: Anti-CD20 Amatoxin Conjugates with Non-Cleavable Linker

### Example 2.1: Synthesis of Anti-CD20 Amatoxin Conjugates with Non-Cleavable Linker

### Step 1: 6′-O-(6-Boc-aminohexyl)-α-amanitin (HDP 30.0132)

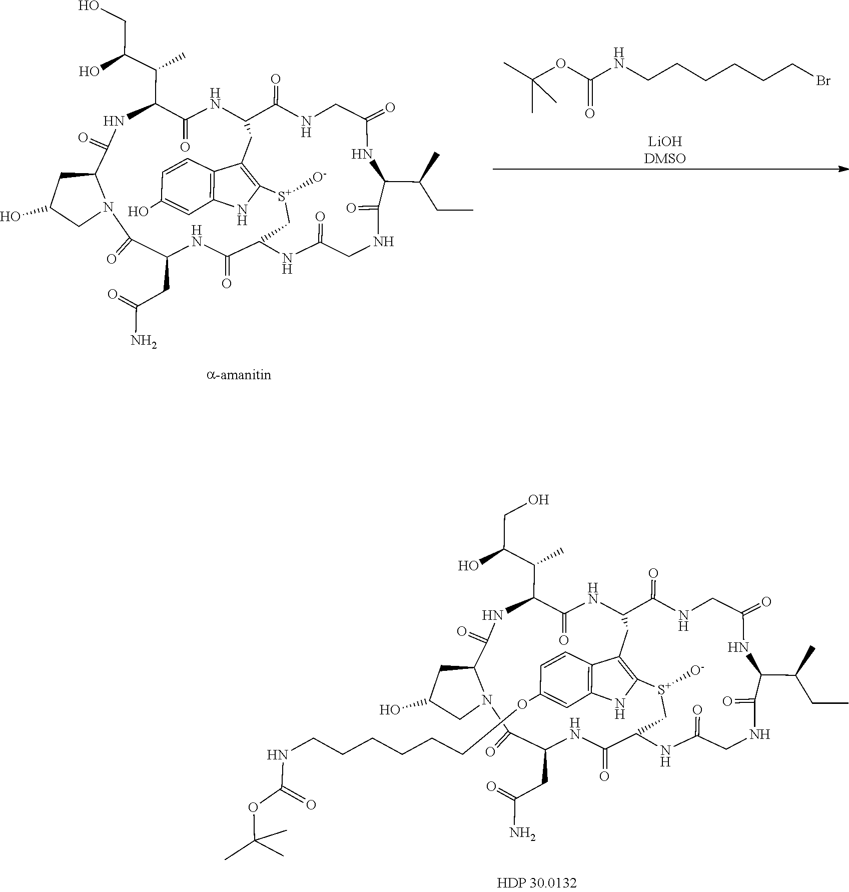

A solution of α-amanitin (105 mg, 114 μmol) and 6-(Boc-amino)-hexyl bromide (128 mg, 457 μmol) in DMSO (3.5 mL) was treated with a 2 M lithium hydroxide (LiOH) solution (68.6 μl, 137.1 μmol) under argon atmosphere. After stirring at ambient temperature for 40 min, the reaction mixture was acidified by addition of AcOH (7.84 μl) and then the mixture was added drop wise to a flask containing MTBE (40 mL) in order to precipitate the desired ether intermediate. The supernatant was decanted and discarded. The precipitate was purified by preparative RP-HPLC [λ=305 nm; gradient: 0-5 min 5% B; 20-25 min 100% B; 27-35 min 5% B; A=water; B=methanol] to provide HDP 30.0132 (84.37 mg, 66%) as a white powder.

MS (ESI+): m/z found: 1118.5 calc.: 1119.29 [M+H]⁺.

### Step 2: 6′-O-(6-aminohexyl)-α-amanitin (HDP 30.0134)

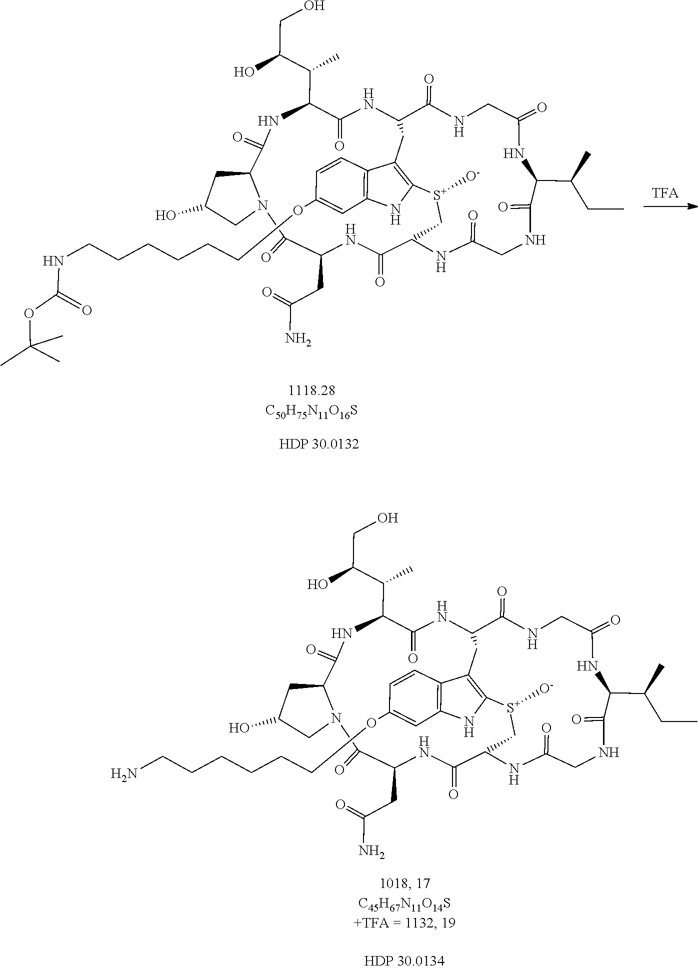

To HDP 30.0132 (152 mg, 136 μmol) TFA (5 mL) was added and the reaction mixture was stirred for 2 min at ambient temperature. The reaction mixture was concentrated under reduced pressure, and the crude product was purified by preparative RP-HPLC [λ=305 nm; gradient: 0 min 5% B; 0-1 min 30% B; 1-10 min 39% B; 10-13 min 100% B; 13-18 min 5% B; A=water with 0.05% TFA; B=methanol with 0.05% TFA]. The fractions containing the product were combined, concentrated and lyophilized to yield the derivative HDP 30.0134 (118.67 mg, 86%).

MS (ESI+): m/z found: 1018.5 calc.: 1019.17 [M+H]⁺.

### Step 3: 6′-O-(6-aminohexyl)-α-amanitin N-succinimidyl carbamate HDP 30.0643

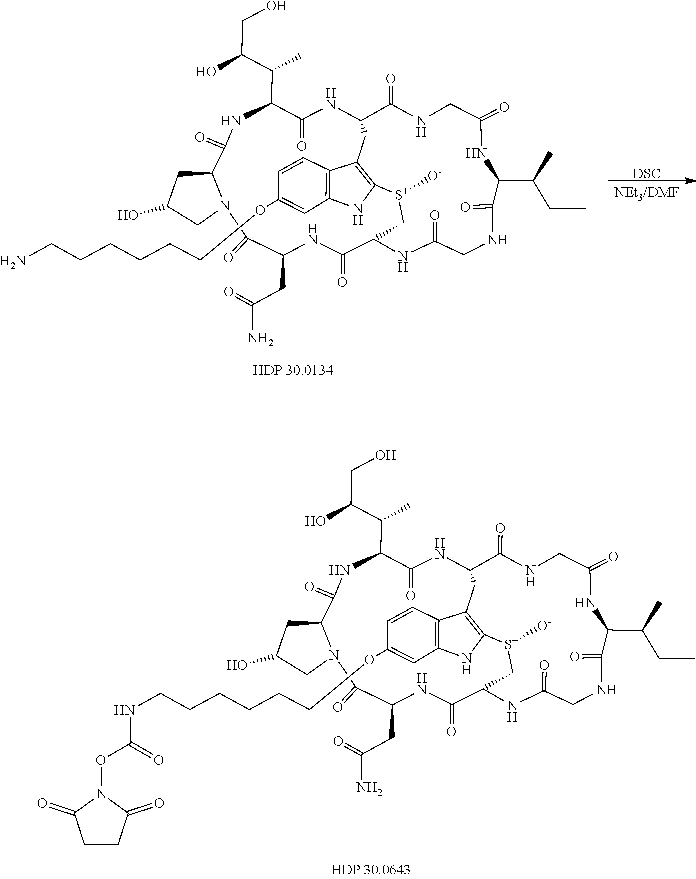

Step 2 product, 207 mg (183 μmol) HDP 30.0134 was dissolved and transferred into a 50 ml conical centrifuge tube with 4000 μl dry dimethylformamide (DMF).

A 0.2 M solution of N,N′-Disuccinimidyl carbonate was prepared by dissolving 128 mg (500 μmol) DSC in 2500 μl DMF and 1828 μl (366 μmol=2 equivalents) of the resulting solution was added to HDP 30.0134 followed by 50.7 μl (366 μmol=2 eq.) triethylamine.

After vortexing, the centrifuge tube was placed on an orbital shaker at ambient temperature. TLC control after 5 min indicated complete consumption of starting material. Subsequently 40 ml ice-cooled MTBE and 20 μl TFA added to the tube. After vigorous vortexing the tube was placed for 10 min in an ice bath and the precipitate was centrifuged at 4000×g for 3 min. The supernatant was decanted and the pellet was washed with 10 ml ice-cooled 0.05% TFA in MTBE by means of resuspension and sedimentation. The solid was dried in vacuo, redissolved in 2400 μl water/methanol 5:95+0.05% TFA purified by preparative HPLC.

Product containing fractions were combined and evaporated. The residue is dissolved in 10 ml of a mixture of tert-butanol and water 4:1 containing 0.05% TFA. The solution was passed through a syringe filter (nylon 0.2 μm, Ø 30 mm) and freeze-dried:
170 mg (80%) colorless powder

MS (ESI+): 1159.42; calc for MH+. (C50H71N12O18S): 1159.47.

### Step 4a: Conjugation of 6′-O-(6-aminohexyl)-α-amanitin to Rituximab

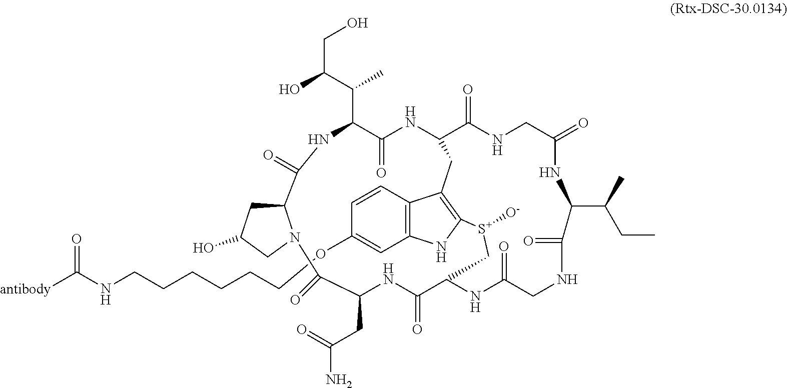

Variant A: In Situ Activation with DSC and HDP 30.0134

The anti-CD20 monoclonal antibody rituximab was conjugated to compound I by use of the coupling reagent DSC (N,N′-Disuccinimidyl-Carbonate) yielding a rituximab-amatoxin conjugate with a non-cleavable linker connecting the 6′-position of the indol system of amino acid 4 of amatoxin to lysine residues of rituximab (Rtx-DSC-30.0134) as follows:

0.66 mg 6′-(−6-aminohexyl)-α-amanitin HDP 30.0134 were dissolved in 72 μl dry dimethylformamide (DMF). Under argon and stirring at room temperature 6.7 μl of a solution of dihydroxysuccinimido carbonate (DSC) in DMF (2.56 mg in 100 μl DMF) and 1.3 μl triethylamine were added at once. The reaction mixture was stirred at room temperature. After 12 h, 30 ml cold diethyl ether were added. The precipitate of α-amanitin-6′-(−6-aminohexyl-6-hydroxysuccinimidyl carbonate) was collected and washed several times with diethyl ether and dried in vacuum. The remaining solid was taken up in 100 μl DMF.

4.0 μl of the above prepared DMF solution was added to 225 μl of a rituximab (Roche) solution (2.0 mg/ml in phosphate buffered saline (PBS).

The mixture was shaken at 4° C. for 14 h and separated by Sephadex G25 gelfiltration on a PD-10 column. Protein fractions were detected by UV absorption and concentrated on Vivaspin Centrifugal Concentrators at 3000 g. Protein concentration was determined by RotiQuant-Assay (Carl Roth; Germany) and adjusted to 2.0 mg/ml. Amanitin payload of Rituximab was determined by UV-absorption at 280 nm and 310 nm, using the extinction coefficients of antibodies and α-amanitin to yield a drug antibody ration (DAR) of 2.6.

### Variant B: Use of Preactivated HDP 30.0643

0.90 mg 6′-O-(6-aminohexyl)-α-amanitin N-succinimidyl carbamate HDP 30.0643 was dissolved in 180 μl dry dimethyl sulfoxide (DMSO) and 165 μl of the resulting solution was added immediately to 2 ml of rituximab (6 mg/ml in PBS).

The mixture was shaken at 4° C. overnight and subsequently separated by Sephadex G25 gelfiltration on a PD-10 column. Protein fractions were detected by UV absorption and concentrated on Amiconspin Centrifugal Concentrators at 2000 g. Protein concentration was determined by Bradford-Assay and adjusted to 3.0 mg/ml.

Amanitin payload of Rituximab was determined by UV-absorption at 280 nm and 310 nm, using the extinction coefficients of antibodies and α-amanitin. to yield a drug antibody ratio (DAR) of 4.6.

### Step 4b: Conjugation of 6′-O-(6-aminohexyl)-α-Amanitin with Reducible DSP Linker to Rituximab

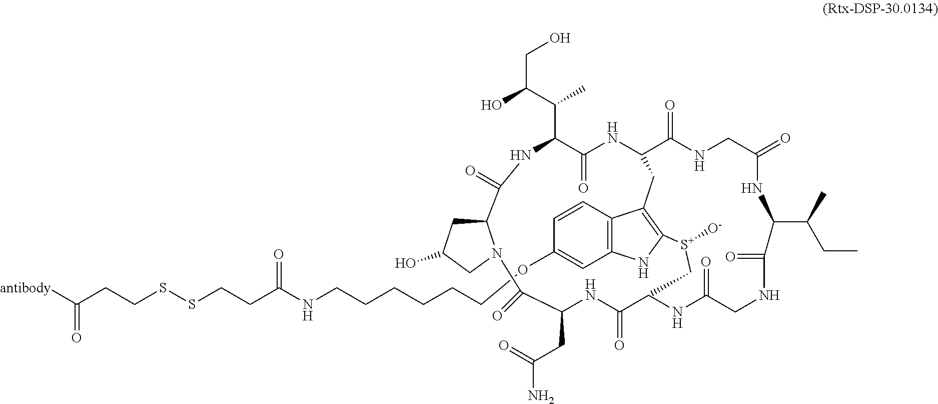

The anti-CD20 monoclonal antibody rituximab was also conjugated to step 2 product (HDP 30.0134) by use of the coupling reagent DSP (Dithiobis-succinimidyl-proprionate) yielding a rituximab-amatoxin conjugate with a disulfide-containing linker connecting the 6′-position of the indol system of amino acid 4 of amatoxin to lysine residues of rituximab (Rtx-DSP-30.0134) as follows:

1.0 mg 6′-(−6-aminohexyl)-cx-amanitin HDP 30.0134 were dissolved in 56.6 μl dry dimethylformamide (DMF). Under argon and stirring at room temperature, 12 μl of a solution of (dithiobis(succinimidyl propionate (DSP) in DMF (3.7 mg in 100 μl DMF) and 2.8 μl triethylamine were added at once. The reaction mixture was stirred at room temperature. After 18 h, 30 ml cold diethyl ether were added. The precipitate was collected and washed several times with diethyl ether and dried in vacuum. The remaining solid was taken up in 100 μl DMF.

Three samples of 500 μl rituximab (Roche) solution (2.0 mg/ml) in phosphate buffered saline (PBS) were treated with 4.2, 14.7 and 29.4 μl of the above prepared DMF solution corresponding to a 1-, 3.5- and 7.0-fold molar excess of toxin linker.

The mixtures were shaken at 4° C. for overnight and separated each by Sephadex G25 gelfiltration chromatography (XK-16 column; 2 ml/min). Conjugate fractions were detected by UV absorption and concentrated on Vivaspin Centrifugal Concentrators at 3000 g. Protein concentration was determined by RotiQuant-Assay (Carl Roth; Germany) and adjusted to 3.0 mg/ml.

Amanitin payload of Rituximab was determined by UV-absorption at 280 nm and 310 nm, using the extinction coefficients of antibodies and α-amanitin to yield drug antibody ratios (DAR) of 0.9, 4.4 and 7.0.

### Example 2.2: Cytotoxicity of Anti-CD20 Amatoxin Conjugates with Non-Cleavable Linker in Vitro

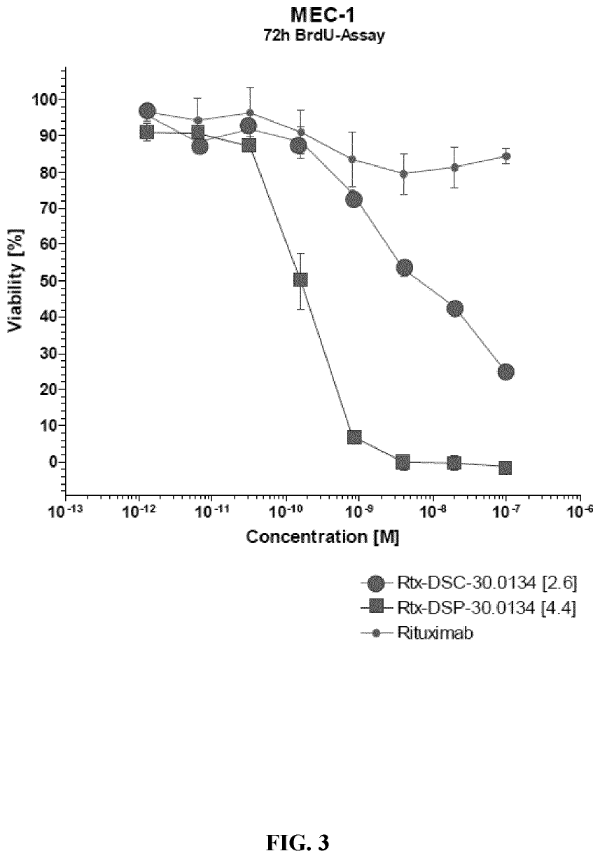

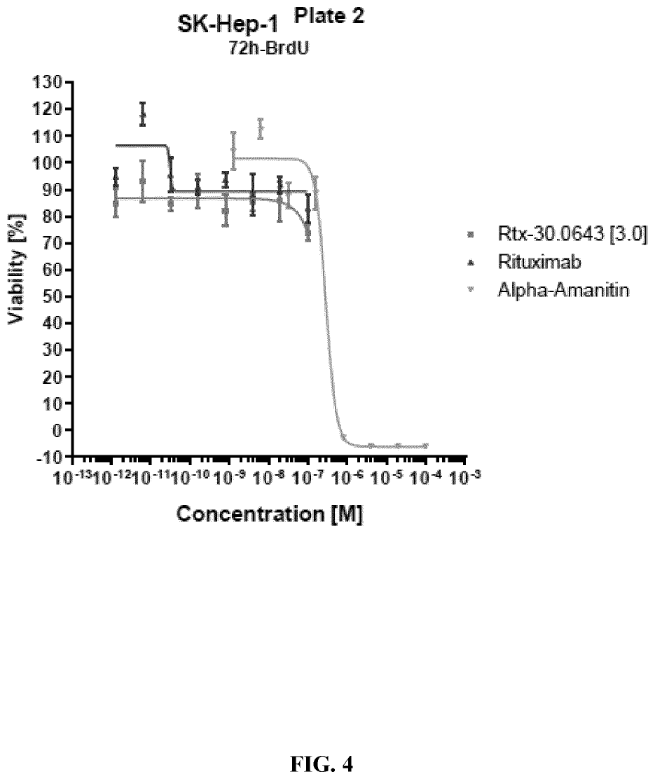

Cytotoxic activity of the rituximab-amatoxin conjugates Rtx-DSC-30.0134 and Rtx-DSP-30.0134 was assessed in vitro on the human chronic B-cell leukemia cell line MEC-1 using a chemiluminescent BrdU-ELISA incorporation assay according to the protocol of the manufacturer (Roche). Unconjugated rituximab was used as a control. Results are depicted in FIG. 3.

Both rituximab-amatoxin conjugates Rtx-DSC-30.0134 and Rtx-DSP-30.0134 (non-cleavable linker and disulfide-containing linkers, respectively) showed significant cytotoxic activity on CD20-positive cells in vitro.

### Example 2.3: Cytotoxicity of Anti-CD20 Amatoxin Conjugate with Non-Cleavable Linker in Vitro as Compared to Anti-EGF-R Amatoxin Conjugates

The anti-CD20 monoclonal antibody rituximab was conjugated to compound I (see Example 2.2) by use of the coupling reagent DSC (N,N′-Disuccinimidyl-Carbonate) yielding a rituximab-amatoxin conjugate with a non-cleavable linker connecting the 6′-position of the indol system of amino acid 4 of amatoxin to lysine residues of rituximab (Rtx-30.0643). This conjugate did not show any significant cytotoxicity when tested on the CD20-negative cell line SK-Hep-1, which is a hepatoma cell line of sinusoidal-endothelial origin (FIG. 4).

Furthermore, the anti-epidermal growth factor receptor (EGF-R) monoclonal antibodies trastuzumab and panitumumab, respectively, were conjugated to compound I (see Example 2.2) by use of the coupling reagent DSC (N,N′-Disuccinimidyl-Carbonate) yielding an antibody-amatoxin conjugate with a non-cleavable linker connecting the 6′-position of the indol system of amino acid 4 of amatoxin to lysine residues of trastuzumab and panitumumab, respectively (Her-30.0643 and Pan-30.0643, respectively).

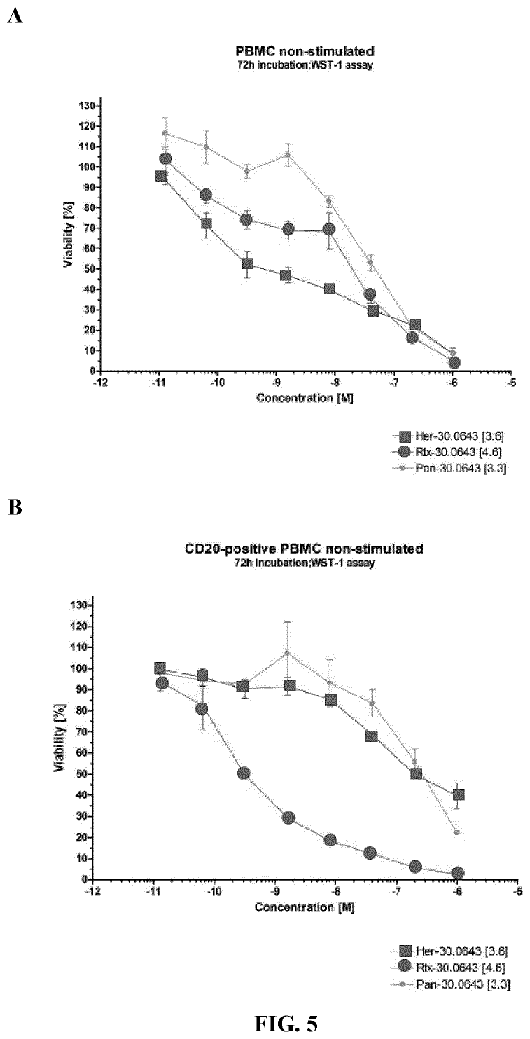

Cytotoxic activities of the rituximab-, trastuzumab- and panitumumab-amatoxin conjugates, respectively, were assessed in vitro on non-stimulated peripheral blood mononuclear cells (PBMC) using a WST-1 assay. All three conjugates (Rtx-30.0643, Her-30.0643 and Pan-30.0643) showed cytotoxic effects in the WST-1 assay, wherein the dose-response curves with all three conjugates were found to cover rather broad concentration ranges (FIG. 5, upper panel). Her-30.0643 was found to exert the strongest cytotoxicity.

When cytotoxic activities of the different conjugates were assessed on CD20-enriched non-stimulated PBMCs (FIG. 5, lower panel), the CD20-specific conjugate Rtx-30.0643 was considerably more cytotoxic, having an IC₅₀ of ca. 2×10⁻¹⁰ M, than the two other conjugates Her-30.0643 and Pan-30.0643, having an IC₅₀ of ca. 3×10⁻⁷ M each.

### Example 2.4: Cytotoxicity of Anti-CD20 Amatoxin Conjugate with Non-Cleavable Linker in Vitro as Compared to Anti-CD20-F(ab′)₂ Fragment Amatoxin Conjugate

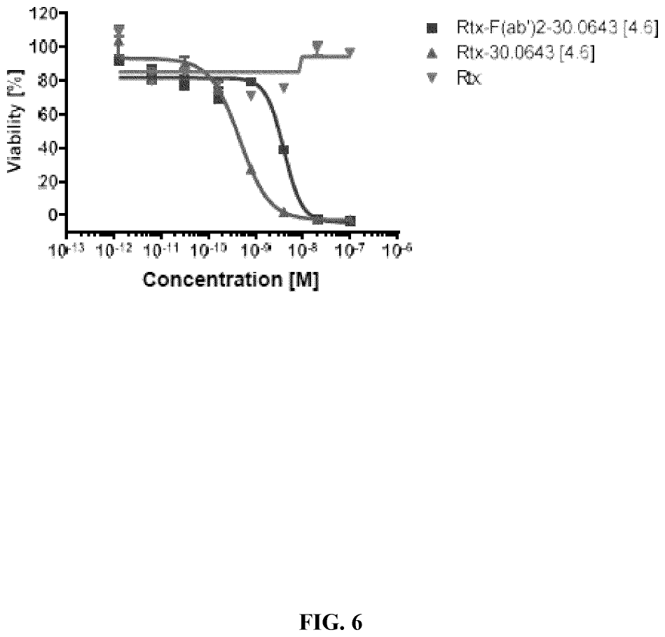

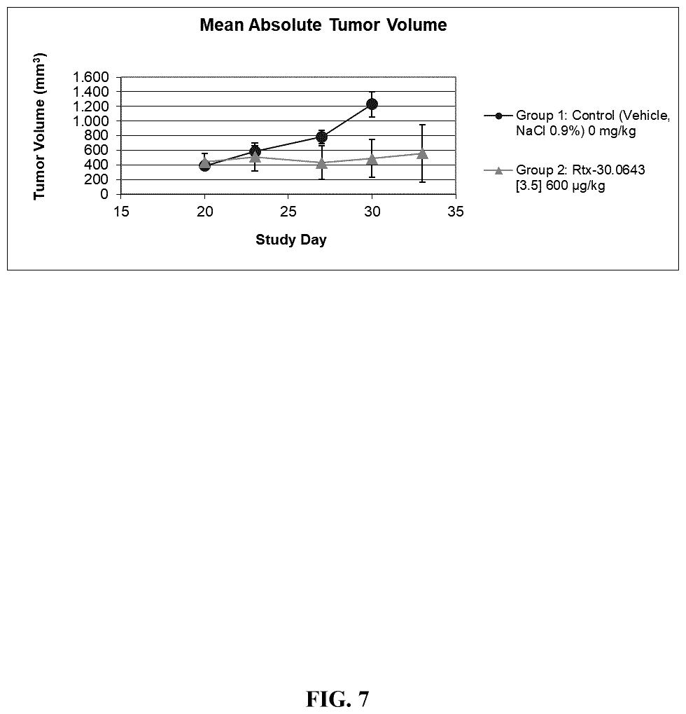

In addition to the rituximab-amatoxin conjugate Rtx-30.0643, a rituximab-F(ab′)₂ fragment amatoxin conjugate (Rtx-F(ab′)₂-30.0643) was generated as described in Example 2.2 step 4. Both conjugates and unconjugated rituximab were assessed for cytotoxicity on CD20-positive MEC-1 cells in vitro using a chemiluminescent BrdU-ELISA incorporation assay. Results are depicted in FIG. 6. Both conjugates showed a significant cytotoxic effect on MEC-1 cells, having an IC₅₀ of ca. 4.6×10⁻¹⁰ M (Rtx-30.0643) and 4×10⁻⁹ M (Rtx-F(ab′)₂-30.0643).

Studies using the WST-1 cytotoxicity assay on MEC-1 cells yielded similar results, with IC₅₀ of ca. 4.1×10⁻¹⁰ M (Rtx-30.0643) and 2.9×10⁻⁹ M (Rtx-F(ab′)2-30.0643).

### Example 2.5: Anti-Tumor Activity of Anti-CD20 Amatoxin Conjugates with Non-Cleavable Linker In Vivo

The rituximab-amatoxin conjugate Rtx-30.0643 having a non-cleavable linker was also tested for cytotoxicity in vivo in a SCID beige-based murine tumor model (FIG. 7). The dose used was 28 mg/kg of rituximab-amatoxin conjugate, relating to a dose of 600 μg/kg of amanitin. The conjugate showed a cytotoxic effect preventing any significant increase in tumor volume over the study period.

### Example 3: Exploratory Toxicity Study of Anti-CD20 Amatoxin Conjugate with Noncleavable Linker in Cynomolgus Monkeys

The rituximab-amatoxin conjugate Rtx-30.0643 (with a payload of 3.2 amanitin moieties per IgG molecule) having a non-cleavable linker was tested in an exploratory toxicity study in Cynomolgus monkeys (Macaca fascicularis); unconjugated rituximab was used as a reference (control). Six male animals at the age of 3.6 to 4.2 years and with a body weight of 3.6 to 4.2 kg at first dosing were used. Parameters assessed in the study included local tolerance, mortality, clinical signs, body weight, hematology (HGB, RBC, WBC, differential blood count (rel., abs.), Reti, PCT, HCT, MCV, MCH, MCHC), coagulation (TPT, aPTT, ESR), clinical biochemistry (albumin, globulin, albumin/globulin ratio, cholesterol (total), bilirubin (total), creatinine, glucose, protein (total), urea, triglycerides, electrolytes, ALAT, aP, ASAT, LDH, CK, gamma-GT, GLDH).

TABLE 2
Experimental Groups

| | | | Dose | | | |
|---|---|---|---|---|---|---|
| | | | [Amanitin | [IgG | No. of | |
| | Test | Administration | μg/kg | mg/kg | male | Animal |
| Group | item | on test day | b.w.] | b.w.] | animals | no. |
| 1 | Rituxan | 1 | 0 | 0.050 | 3 | 1-3 |
| | | 8 | 0 | 0.150 | | |
| | | 15 | 0 | 0.450 | | |
| | | 22 | 0 | 1.250 | | |
| 2 | Rituxan | 1 | 1 | 0.050 | 3 | 1-3 |
| | 30.0643 | 8 | 3 | 0.150 | | |
| | | 15 | 9 | 0.450 | | |
| | | 22 | 25 | 1.250 | | |

TABLE 3
Study Schedule

| Study day | 1 | 6 | 8 | 13 | 15 | 20 | 22 | 27 | 34 | 35 |
|---|---|---|---|---|---|---|---|---|---|---|
| Treatment | x | | x | | x | | x | | | |
| FACS | | x | | x | | x | | x | x | |
| Analysis | | | | | | | | | | |
| Hema- | predose | x | | x | | x | | x | x | |
| tology | | | | | | | | | | |
| Clinical | predose | x | | x | | x | | x | x | |
| Chemistry | | | | | | | | | | |
| Body | predose | | predose | | predose | | predose | | | |
| weight | | | | | | | | | | |
| Plasma | predose | x | | x | | x | | x | x | |
| Dissection | | | | | | | | | | x |

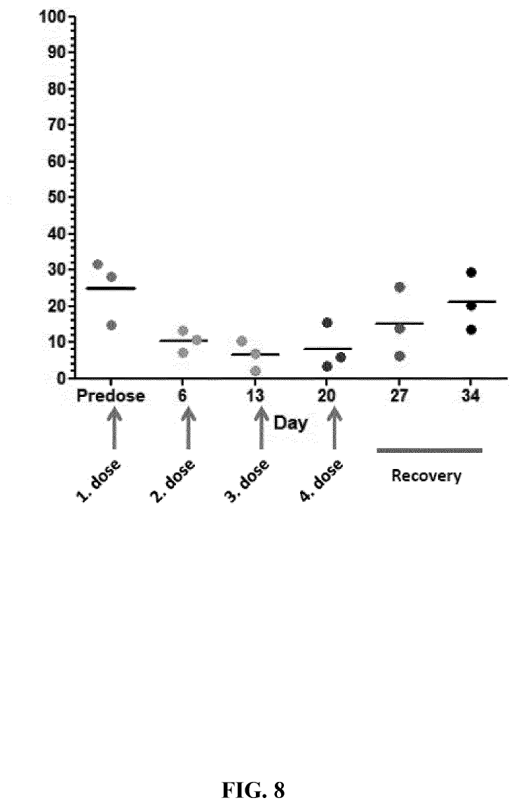

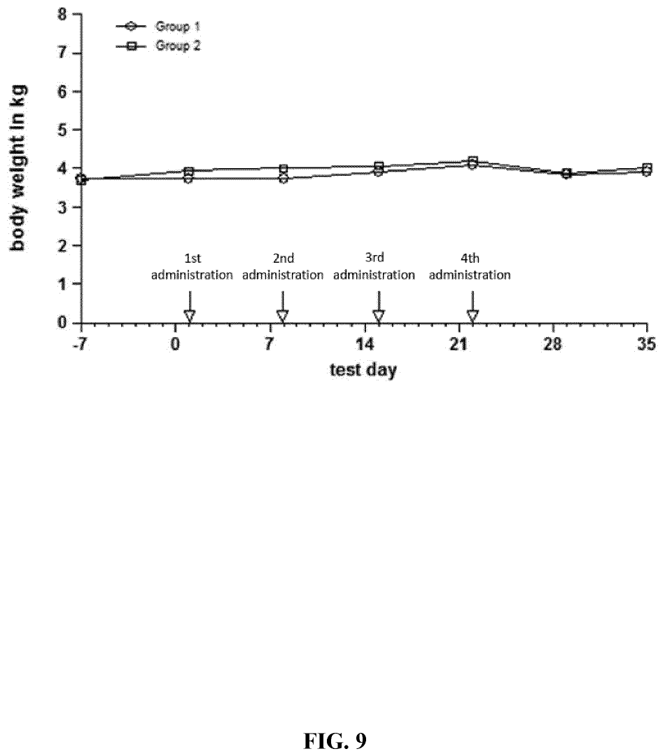

In study group 2 receiving the rituximab-amatoxin conjugate Rtx-30.0643, but not in study group 1 receiving rituximab, a significant B cell depletion was observed, followed by recovery of B cell counts after the last treatment. Results of the study are depicted in FIG. 8.

Body weights remained constant in both study groups over the study period (FIG. 9). No findings were made in the course of the study with regard to organ weights, histopathology (heart, liver, spleen, kidneys, ureter) and macroscopic post-mortem observations.

With regard to hematology, an increase of aPTT, monocytes and basophilic granulocytes were observed on study day 13, five days after treatment at a dosage of 3 μg/kg; until the end of the study, aPTT had returned to normal; monocytes und basophilic granulocytes were increased in study group 2 in comparison to study group 1.

With regard to clinical biochemistry, increased enzyme activities (ALAT, ASAT, LDH, CK, GGT, GLDH) were observed on study 13, five days after treatment at a dosage of 3 μg/kg, for ALAT on study day 20, five days after treatment at a dosage of 9 μg/kg amanitin. Until the end of the study, these parameters had returned to normal.

### Example 4: Anti-CD20 Amatoxin Conjugates with Disulfide Linkers

### Example 4.1: Synthesis of Anti-CD20 Amatoxin Conjugates with Disulfide Linkers

### A. In-Situ Coupling Methods

### Step 1

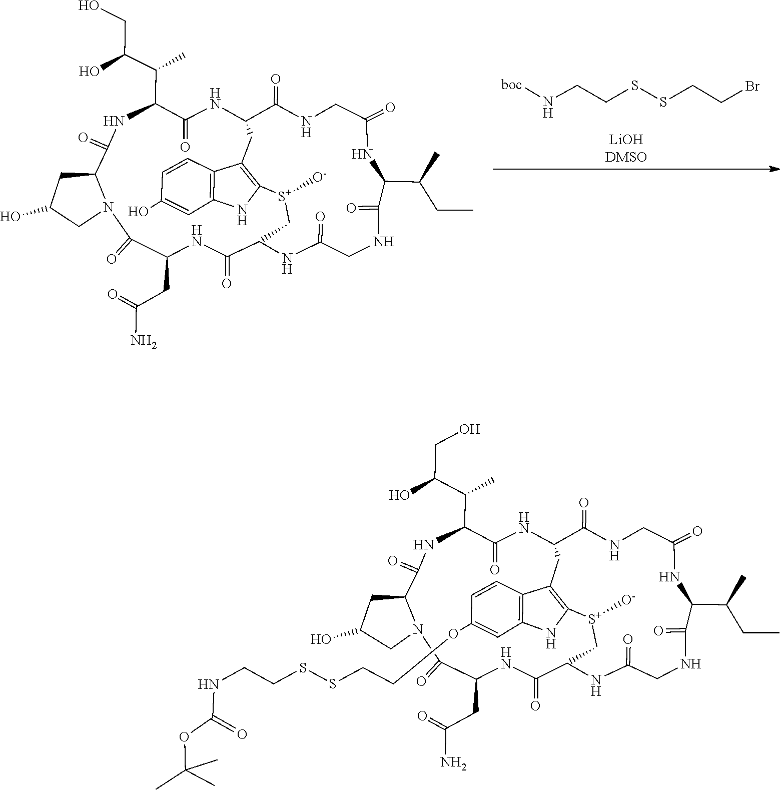

In analogy to Example 2.1 step 1, 5.67 mmol α-amanitin was converted with 2-(2-Bromoethyldisulfanyl)-ethyl]-carbamic acid tert-butyl ester to yield 1.29 mg (15%) of HDP 30.0341 as a white powder.

MS (ESI⁺): m/z found: 1155.2 calc.: 1154.3.5 [M+H]⁺.

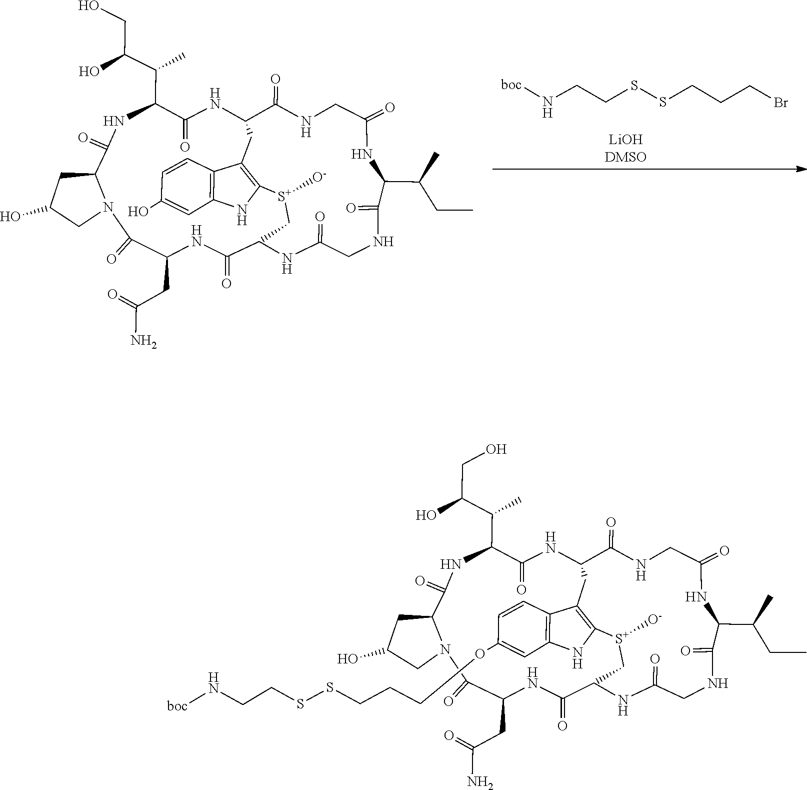

In analogy to Example 2.1 step 1, 5.67 mmol α-amanitin was converted with [2-(3-Bromopropyldisulfanyl)-ethyl]-carbamic acid tert-butyl ester to yield 4.83 (67%) of HDP 30.0349 as a white powder.

MS (ESI⁺): m/z found: 1168.6 calc.: 1168.5 [M+H]⁺.

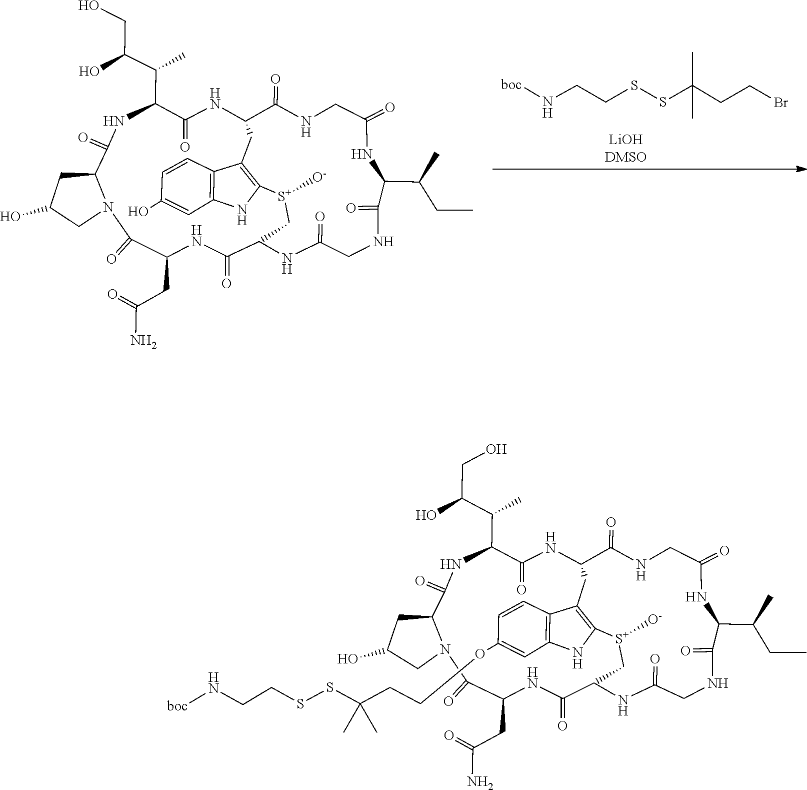

In analogy to Example 2.1 step 1, 5.67 mmol α-amanitin was converted with [2-(3-Bromo-1,1-dimethyl-propyldisulfanyl)-ethyl]-carbamic acid tert-butyl ester to yield 0.51 mg (7%) of HDP 30.0350 as a white powder.

MS (ESI⁺): m/z found: 1196.7 calc.: 1196.5 [M+H]⁺.

### Step 2

In analogy to Example 2.1 step 2, the step 1 products were deprotected to the free amines:

TABLE 4
Yields of process steps

| | | | MS (ESI⁺): m/z | |
|---|---|---|---|---|
| Step 1 | Step 2 | Yield | found | calculated |
| HDP 30.0341 | 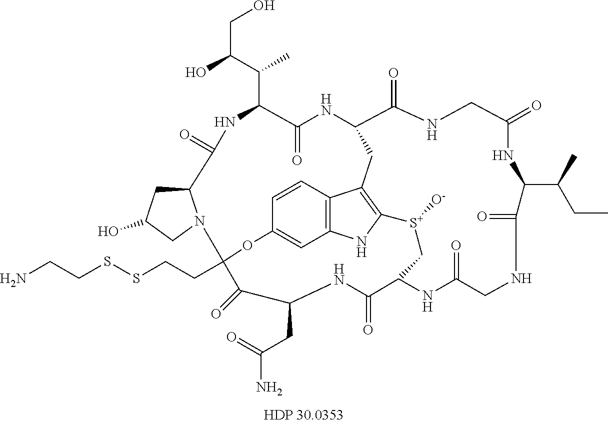 | 64% | 1053.8 | 1054.4 [MH]⁺ |
| HDP 30.0349 | 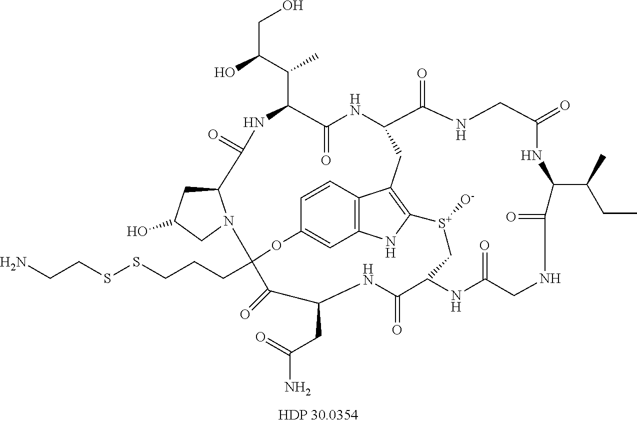 | 55% | 1068.4 | 1068.4 [MH]⁺ |
| HDP 30.0350 | 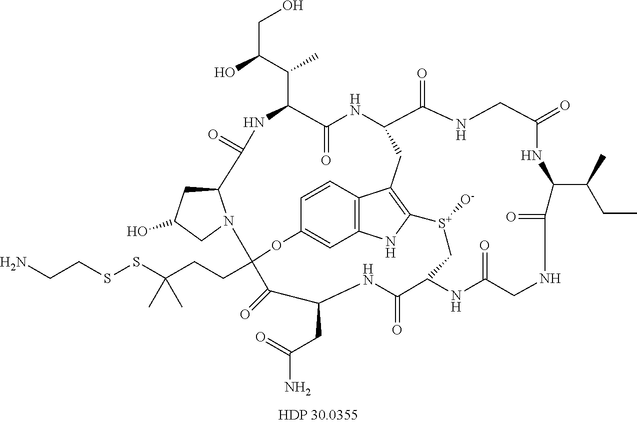 | 28% | 1068.5 | 1096.4 [MH]⁺ |

### Step 3

Amanitin-linker amines HDP 30.0353-5 were in situ preactivated and coupled to rituximab according to the method described in example 2, step 4 variant A to yield the conjugates Rtx-30.0353 [1.6], Rtx-30.0355 [0.7] and Rtx-30.0355 [0.2].

### B. Branched Linkers

### Step 1: 6′-O-(3-S-tritylsulfanyl-propyl)-α-amanitin (HDP 30.0517)

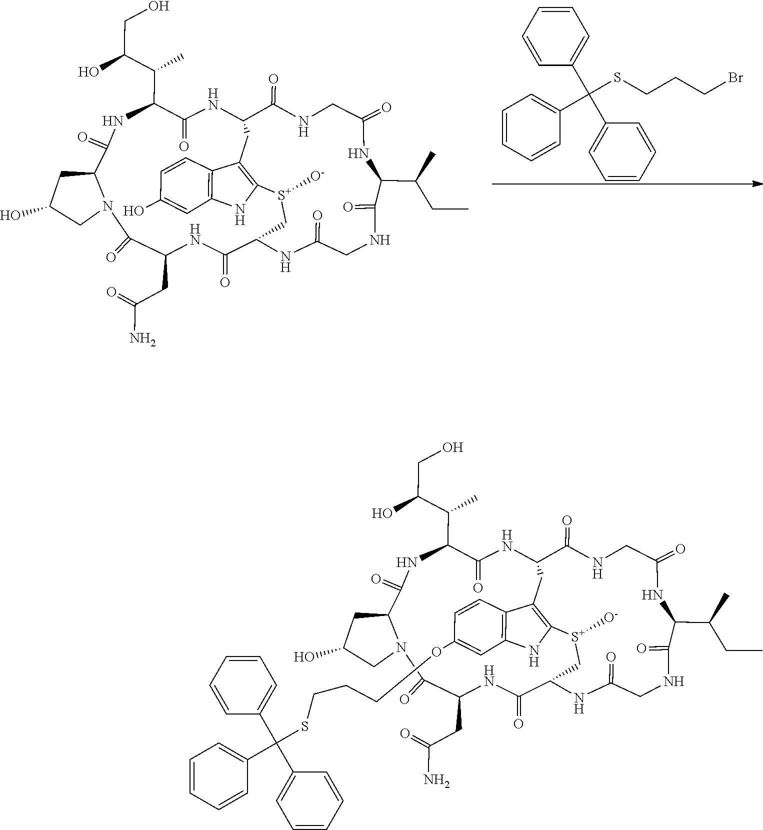

Under argon 46 mg (50 μmol) of vacuum-dried α-amanitin was dissolved in 2500 μl dry DMSO. 3-(S-trityl)-mercaptopropyl-1-bromide (159 mg, 8 eq.) was added, followed by 60 μl of a 1M sodium hydroxide (NaOH) solution. After 1.5 h at room temperature the reaction mixture was acidified to pH 5 with 50 μl 1M AcOH in DMSO and the solvent was evaporated. The residue was dissolved in 200 μl of MeOH and added dropwise to a centrifugation tube filled with 10 ml of MTBE. The resulted precipitate was cooled to 0° C. for 10 min and isolated by centrifugation (4000×g) and washed with 10 ml MTBE subsequently. The supernatants were discarded and the pellet was dissolved in 750 μl of MeOH and purified in 3 portions on preparative HPLC on a C18 column (250×21.2 mm, Luna RP-18, 10 μm, 100 Å) [gradient: 0 min 5% B; 5 min 5% B 20 min 100% B; 25 min 100% B; 27 min 5% B, 35 min 5% B; Flow 30 ml/min]. The fractions with a retention time of 21.1-21.8 min were collected and the solvents evaporated to 36.5 mg (59%) of HDP 30.0517 as a colorless solid.

MS (ESI⁺): m/z found: 1234.8 calc.: 1236.45 [M+H]⁺; found: 1257.3 calc.: 1258.45 [M+Na]⁺.

### 6′-O-(3-S-tritylsulfanyl-butyl)-α-amanitin (HDP 30.1168)

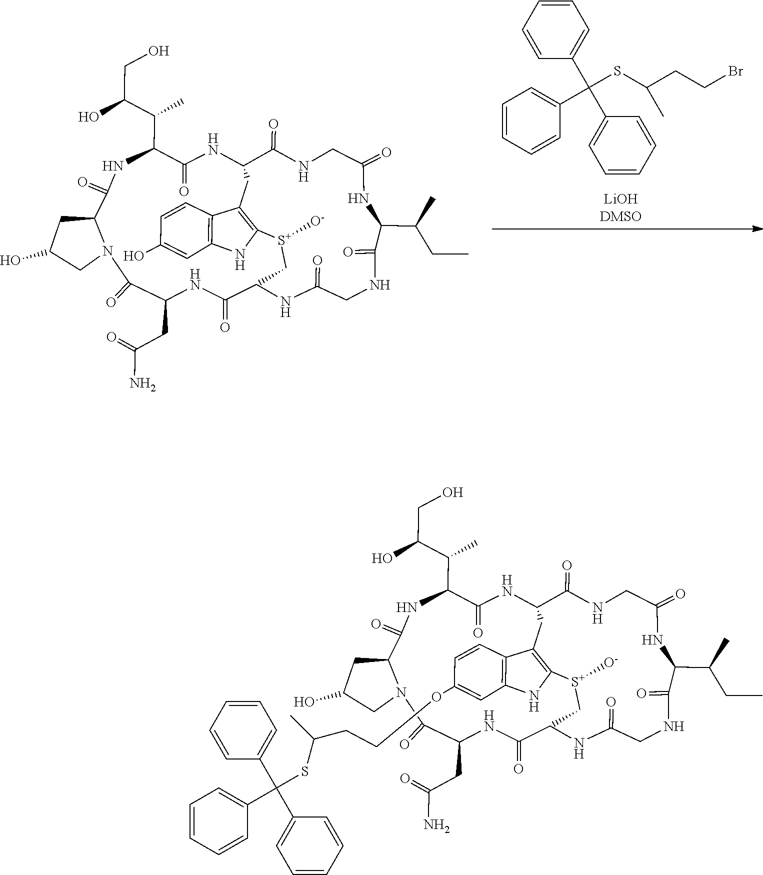

By repeating the above procedure with 3-(S-trityl)-mercaptobutyl-1-bromide, the title product was obtained in 64% yield.

MS (ESI⁺): m/z found: 1271.5 calc.: 1271.5 [M+Na]⁺.

### Step 2: 6′-O-(3-(3-Amino-1-methyl-propyldisulfanyl)-propyl)-α-amanitin (HDP 30.1214)

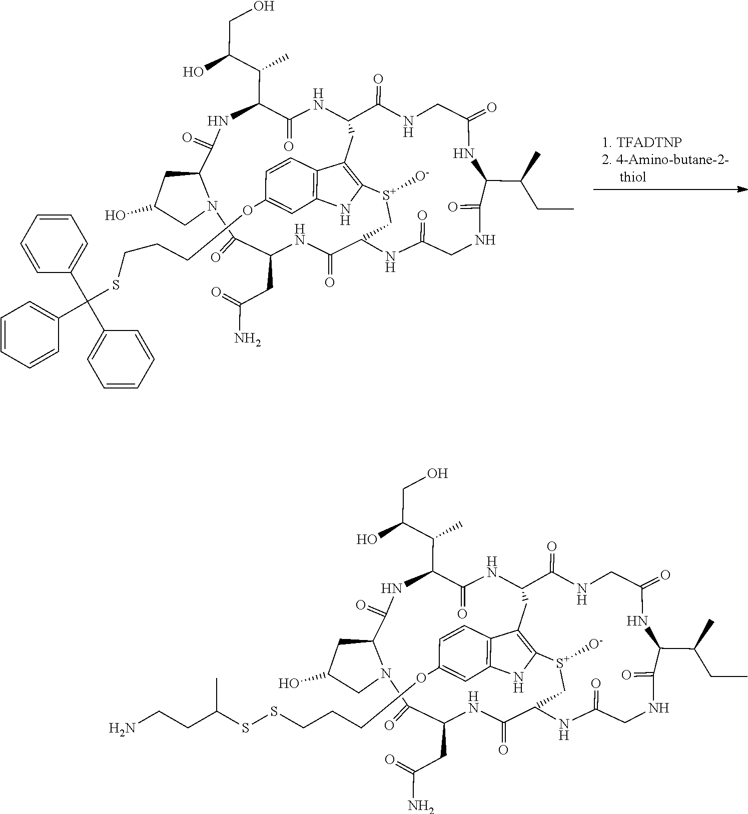

Step 1 product (10 mg) was weighted into a 15 ml centrifuge tube and dissolved in 0.5 M DTNP solution in TFA (80.94 μl, 5 eq). Reaction mixture was stirred at room temperature for 4 minutes. Reaction mixture was then diluted with MTBE/n-hexane (1:1, 10 ml). The precipitate was cooled to 0° C. for 10 minutes, isolated by centrifugation (4000×g) and washed with MTBE (10 ml) subsequently. The supernatants were discarded and the pellet dissolved in 500 μl of MeOH. 4-amino-thiol HDP 30.1157 (17 mg, 9 eq) was added. After 1 h, the mixture was triturated with MTBE with 0.05% TFA (10 ml), the ether decanted and replaced with fresh MTBE with 0.05% TFA (10 ml). The obtained precipitate was dissolved in MeOH (200 μl) and purified on preparative HPLC on a C18 column (250×21.2 mm, Luna RP-18, 10 μm, 100 Å) [λ=305 nm; gradient: 0-5 min 5% B; 20-25 min 100% B; 27-35 min 5% B; A=water with 0.05% TFA; B=methanol with 0.05% TFA]. The fractions corresponding to the product were collected and the solvents evaporated evaporated to 8.05 mg (81%) of HDP 30.1172 as a white powder.

MS (ESI⁺): m/z found: 1110.39 calcd.: 1110.44 [M+H]⁺.

By repeating the above procedure with combining the step 1 products HDP 30.0517 and HDP 30.1168 with the appropriate thiols, the following additional compounds were obtained:

TABLE 5
Yields of process steps

| Step 1 | | | MS (ESI+): m/z | |
|---|---|---|---|---|
| Thiol | Product | Yield | found | calculated |
| HDP 30.0517 4- Amino-2- methyl- butane-2- thiol | 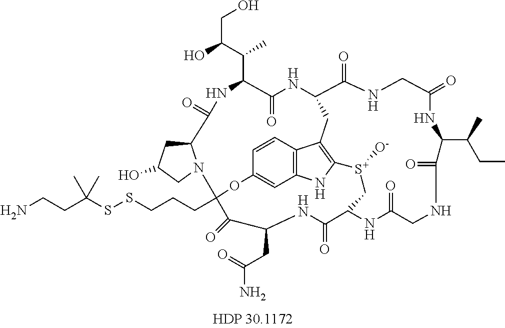 | 81% | 1110.4 | 1110.4 [MH]⁺ |
| HDP 30.1168 cysteamine | 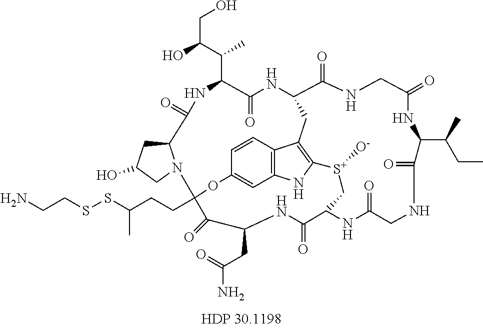 | 80% | 1082.4 | 1082.4 [MH]⁺ |
| HDP 30.1168 4-Amino- butane-2- thiol | 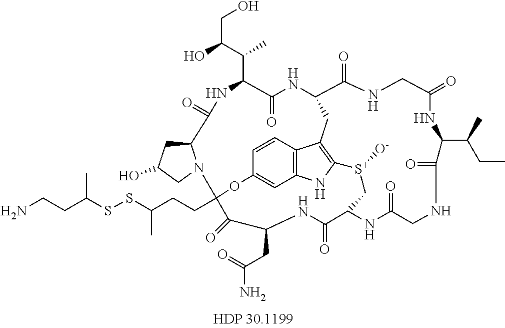 | 61% | 1110.4 | 1110.4 [MH]⁺ |
| HDP 30.1168 4- Amino-2- methyl- butane-2- thiol | 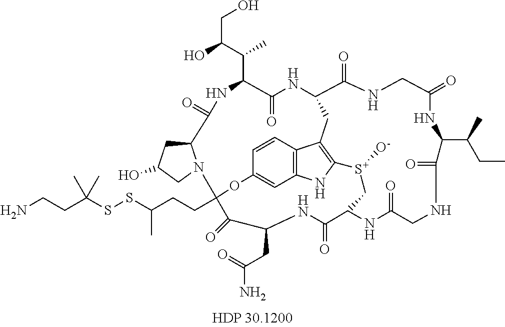 | 76% | 1124.4 | 1124.5 [MH]⁺ |

### Step 3: 6′-O-(3-(3-Amino-1-methyl-propyldisulfanyl)-propyl)-α-amanitin N-succinimidyl Carbamate HDP 30.1214

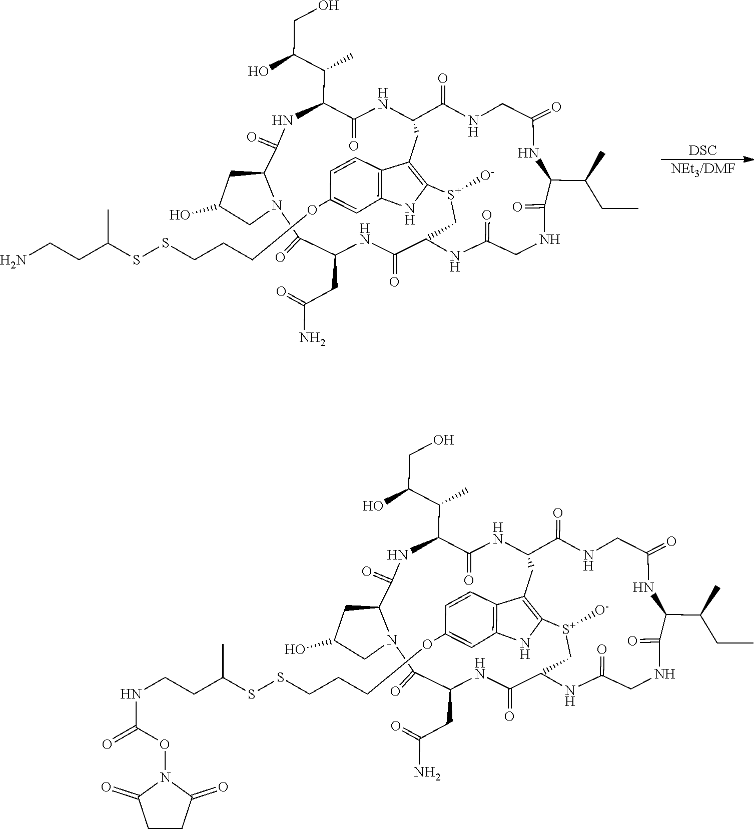

Step 2 product HDP 30.1171, 7.60 mg (6.28 μmol) was dissolved and transferred into a 15 ml conical centrifuge tube with 200 μl dry dimethylformamide (DMF). A 0.2 M solution of N,N′-Disuccinimidyl carbonate was prepared by dissolving 128 mg (500 μmol) DSC in 2500 μl DMF and 314 μl (10 equivalents) of the resulting solution was added to HDP 30.0134 followed by 12.56 μl (366 μmol=2 eq.) 1M triethylamine in DMF. After vortexing, the centrifuge tube was placed on an orbital shaker at ambient temperature. TLC control after 5 min indicated complete consumption of starting material. Subsequently 10 ml ice-cooled MTBE and 5 μl TFA added to the tube. After vigorous vortexing the tube was placed for 10 min in an ice bat and the precipitate was centrifuged at 4000×g for 3 min. The supernatant was decanted and the pellet was washed with 10 ml ice-cold 0.05% TFA in MTBE by means of resuspension and sedimentation. The solid was dried in vacuo redissolved in 2400 μl water/methanol 5:95+0.05% TFA purified by preparative HPLC. Product containing fractions were combined and evaporated. The residue was dissolved in 3 ml of a mixture of tert-butanol and water 4:1 containing 0.05% TFA. The solution is passed through a syringe filter (nylon 0.2 μm, Ø 13 mm) and freeze-dried:
4.68 mg (60%) colorless powder

MS (ESI⁺): 1237.25; calc for MH⁺. (C₅₀H₇₁N₁₂O₁₈S): 1237.43 (C₅₁H₇₃N₁₂O₁₈S₃)

By repeating the above procedure with the variants of step 2, the following additional compounds were obtained:

TABLE 6
Yields of process steps

| | | | MS (ESI+): m/z | |
|---|---|---|---|---|
| Step 2 | Product | Yield | found | calculated |
| HDP 30.1172 | 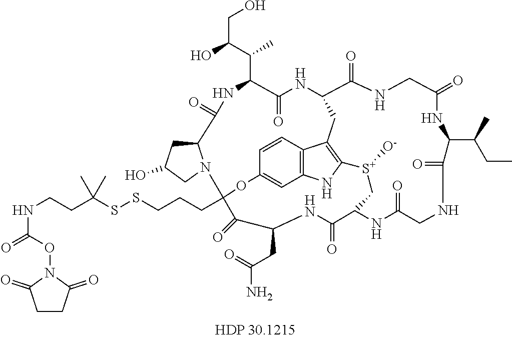 | 61% | 1251.22 | 1251.45 [MH]⁺ |
| HDP 30.1198 | 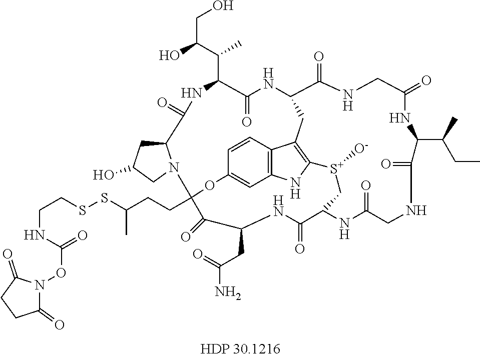 | 79% | 1223.27 | 1223.42 [MH]⁺ |
| HDP 30.1199 | 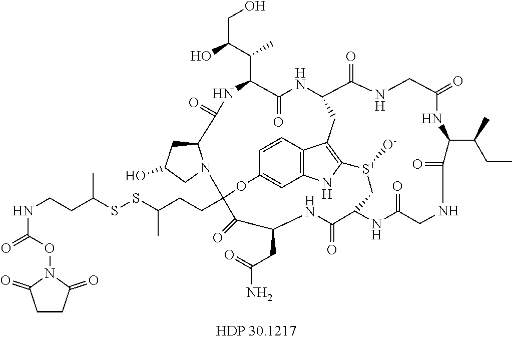 | 63% | 1251.24 | 1251.45 [MH]⁺ |
| HDP 30.1200 | 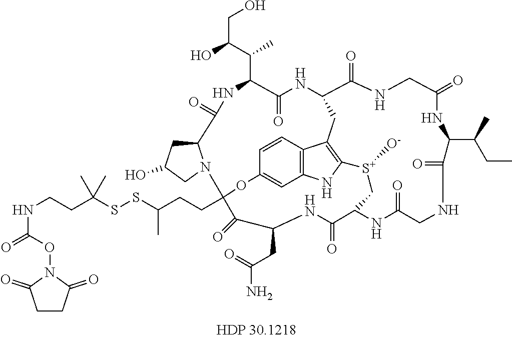 | 61% | 1265.26 | 1265.46 [MH]⁺ |

### Step 4: Synthesis of Rituximab-Amanitin Derivatives with Branched Disulfide Linkers

1.00 mg of each of the succinimidyl carbonate derivative from step 3 were dissolved in 100 μl dry dimethyl sulfoxide (DMSO) and 30 μl (10-fold excess) of each of the resulting solutions were added immediately to 394 μl of a rituximab solution (9.5 mg/ml in PBS). The mixtures were shaken at 4° C. overnight and subsequently separated by Sephadex G25 gelfiltration on a PD-10 column. Protein fractions were detected by UV absorption and dialyzed at 4° C. in Slide-A-Lyzer™ Dialyse Cassettes (MWCO 20′000) against 1 liter of PBS pH 7.4 overnight. Protein concentration was determined by RotiQuant-Assay (Carl Roth; Germany), concentrated on Amiconspin Centrifugal Concentrators at 2000 g and adjusted to 3.0 mg/ml. Amanitin payload of Rituximab was determined by UV-absorption at 280 nm and 310 nm, using the extinction coefficients of antibodies and α-amanitin to yield the following conjugates:

TABLE 7
Product Characteristics

| | | Volume | Concentration | |
|---|---|---|---|---|
| Code | DAR | [ml] | [mg/ml] | Total protein [mg] |
| Rtx-30.0748 | 4.3 | 0.7 | 3.0 | 2.1 |
| Rtx-30.1214 | 4.4 | 0.6 | 3.0 | 1.8 |
| Rtx-30.1215 | 4.6 | 0.6 | 3.0 | 1.8 |
| Rtx-30.1216 | 5.0 | 0.7 | 3.0 | 2.1 |
| Rtx-30.1217 | 4.9 | 0.7 | 3.0 | 2.1 |
| Rtx-30.1218 | 4.6 | 0.6 | 3.0 | 1.8 |

### Example 4.2: Cytotoxicity of Anti-CD20 Amatoxin Conjugates with Disulfide Linkers In Vitro

The anti-CD20 monoclonal antibody rituximab was conjugated to compounds III, IV and V, respectively, by use of the coupling reagent DSC (N,N′-Disuccinimidyl-Carbonate) yielding rituximab-amatoxin conjugates with disulfide linkers connecting the 6′-position of the indol system of amino acid 4 of amatoxin to lysine residues of rituximab (Rtx-DSC-30.0353, Rtx-DSC-30.0354, and Rtx-DSC-30.0355).

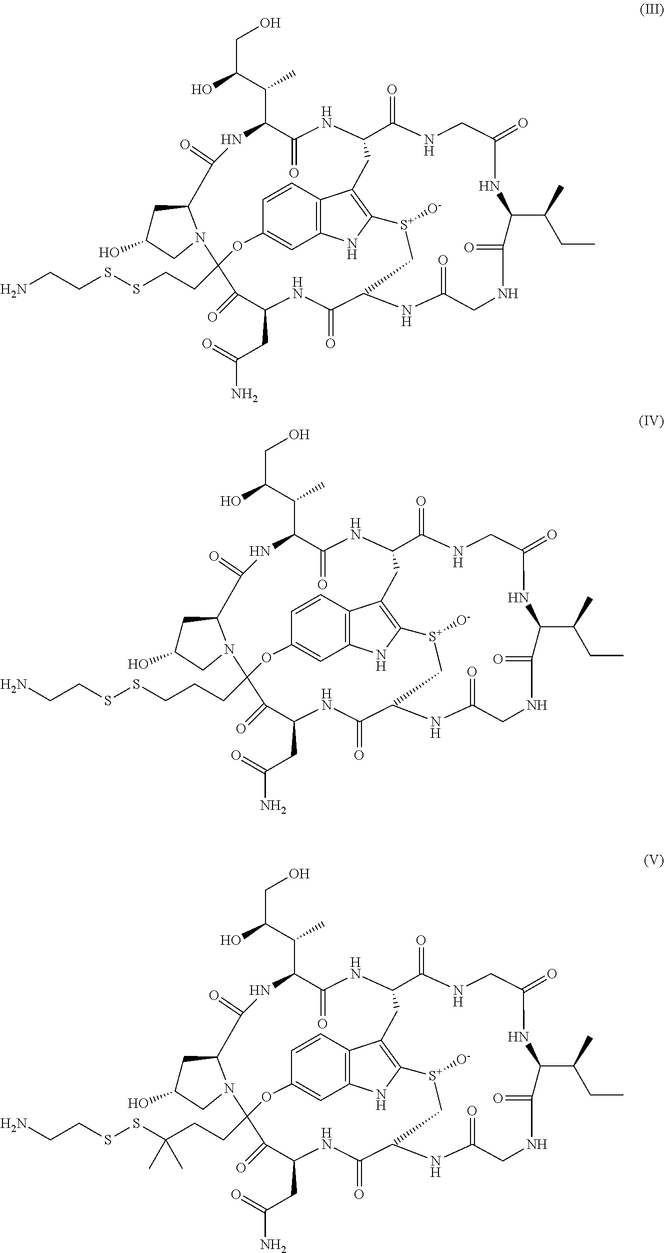

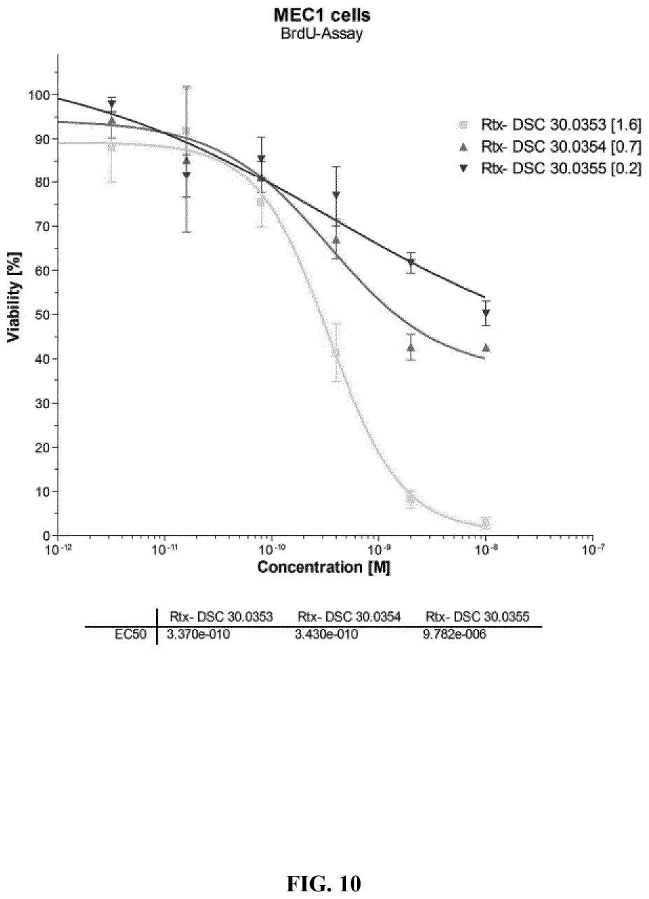

Cytotoxic activities of the rituximab-amatoxin conjugates Rtx-DSC-30.0353, Rtx-DSC30.0354, and Rtx-DSC-30.0355 were assessed in vitro on the human chronic B-cell leukemia cell line MEC-1 using a chemiluminescent BrdU-ELISA incorporation assay according to the protocol of the manufacturer (Roche). Results are depicted in FIG. 10.

The rituximab-amatoxin conjugate Rtx-DSC-30.0353 showed the highest cytotoxic activity on CD20-positive cells in vitro.

Furthermore, anti-CD20 monoclonal antibody rituximab was conjugated to the DSC-preactivated compounds from Example 4.1, yielding rituximab-amatoxin conjugates with disulfide linkers connecting the 6′-position of the indol system of amino acid 4 of amatoxin to lysine residues of rituximab (Rtx-30.0748, Rtx-30.1214, Rtx-30.1215, Rtx-30.1216, Rtx-30.1217 and Rtx-30.1218).

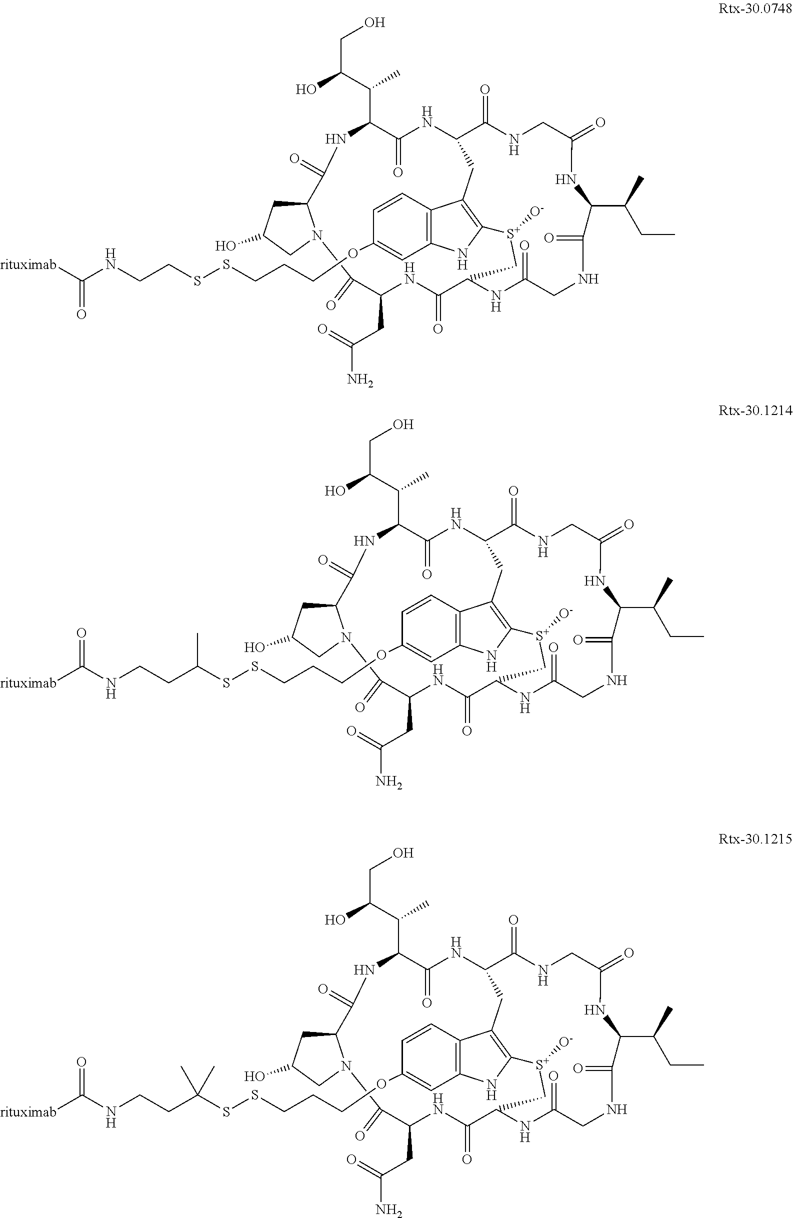

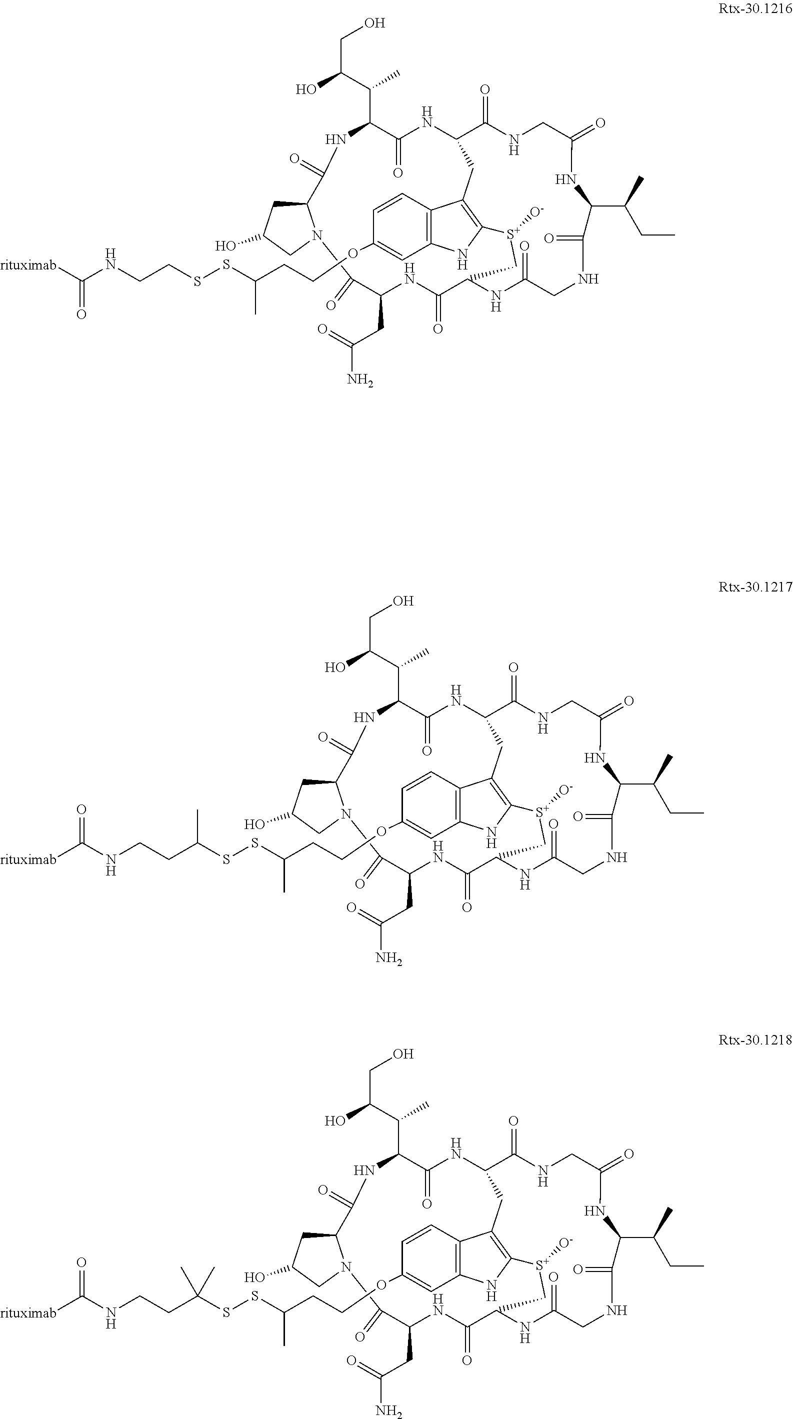

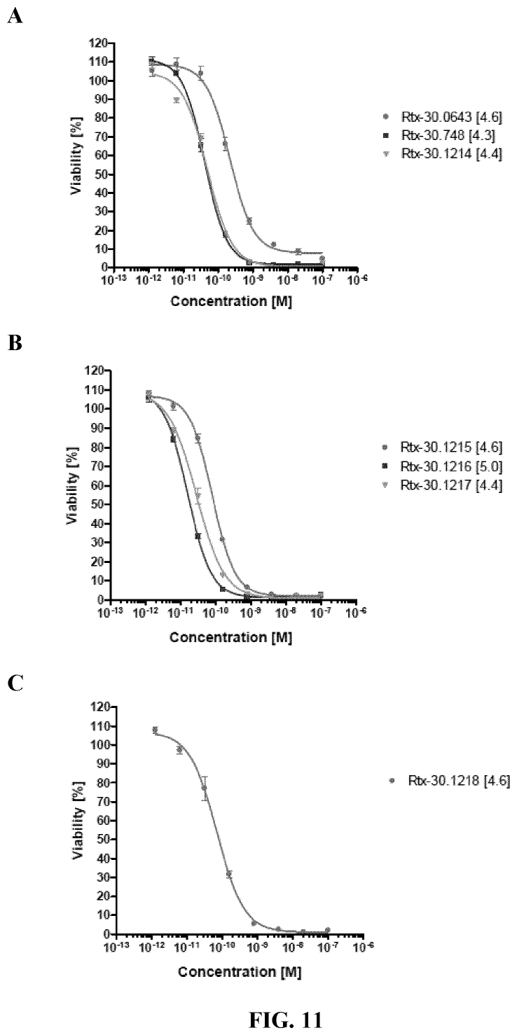

Cytotoxic activities of the rituximab-amatoxin conjugates Rtx-30.0748, Rtx-30.1214, Rtx-30.1215, Rtx-30.1216, Rtx-30.1217 and Rtx-30.1218 were assessed in vitro on the human chronic B-cell leukemia cell line MEC-1 using a WST assay. Results are depicted in FIG. 11.

In the WST assay using MEC-1 cells, cytotoxicity could be shown for all conjugates used. EC₅₀ values of the conjugates Rtx-30.1214, Rtx-30.1215, Rtx-30.1216, Rtx-30.1217 and Rtx-30.1218 were about the same range as the two reference compounds Rtx-30.0643 and Rtx-30.0748, wherein all conjugates were more cytotoxic than Rtx-30.0643 (see Table 3).

The less stabilized disulfides bearing not more than one shielding methyl groups on each side (i.e., Rtx-30.0748, Rtx-30.1217, Rtx-30.1216 and Rtx-30.1214) showed highest cytotoxicity, whereas the highly stabilized Rtx-30.1215 and Rtx-30.1218 were slightly more cytotoxic than Rtx-30.0643 with a non-cleavable linker, indicating limited reductive cleavage of these compounds.

TABLE 8
EC₅₀ Values [M] of rituximab-amatoxin
conjugates with disulfide linkers

| ADC | Cytotoxicity on MEC-1 [M] |
|---|---|
| Rtx-30.1216 | 1.6 × 10⁻¹¹ |
| Rtx-30.1217 | 2.9 × 10⁻¹¹ |
| Rtx-30.0748 | 4.1 × 10⁻¹¹ |
| Rtx-30.1214 | 4.9 × 10⁻¹¹ |
| Rtx-30.1218 | 7.1 × 10⁻¹¹ |
| Rtx-30.1215 | 8.1 × 10⁻¹¹ |
| Rtx-30.0643 | 2.2 × 10⁻¹⁰ |

### Example 5: Rituximab Amatoxin Conjugates with Enzymatically Cleavable Linkers

### Example 5.1: Synthesis of Rituximab Amatoxin Conjugates with Enzymatically Cleavable Linkers

### A: 6′-[(3-maleimidopropanamido)-Val-Ala-PAB]-α-amanitin (HDP 30.1699)

### Step 1: 6′-[Boc-Val-Ala (SEM)-PAB]-α-amanitin (HDP 30.1698)

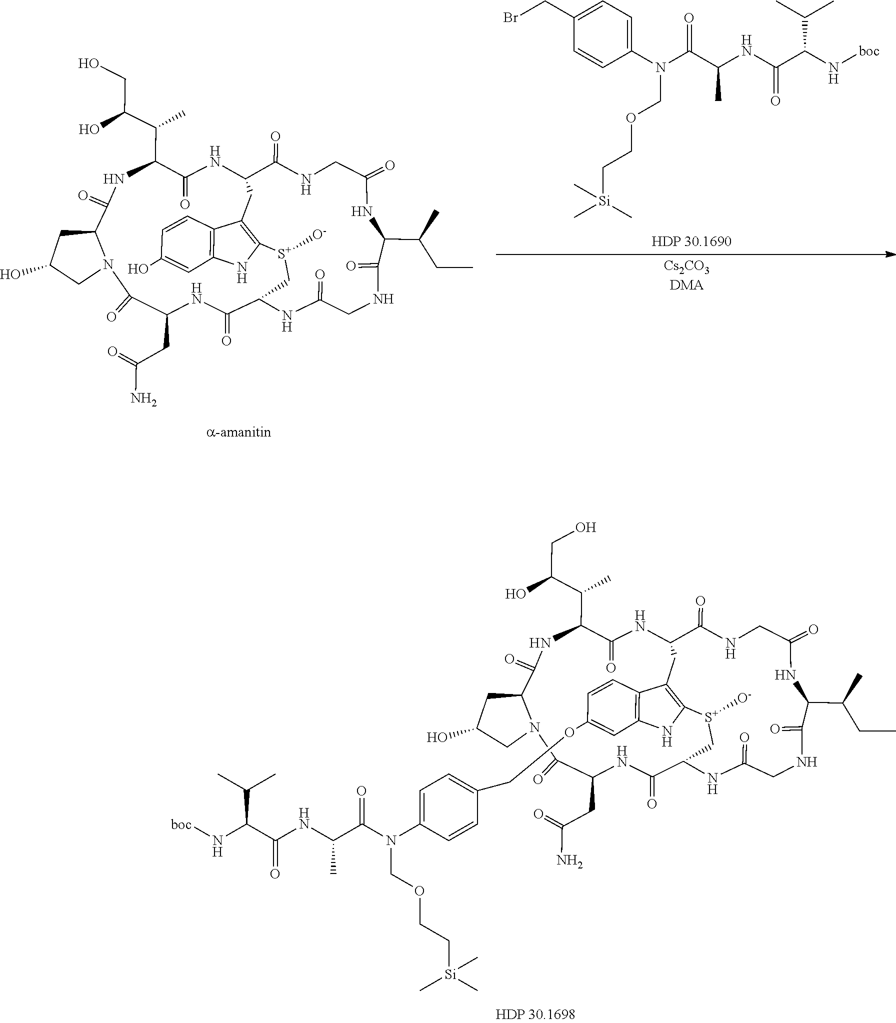

Under argon and at room temperature 57 mg (62.02 μmol) of vacuum-dried α-amanitin were dissolved in 3000 μl dry dimethyl acetamide (DMA). Boc-Val-Ala (SEM)-4-aminobenzyl bromide (disclosed in EP 17192686) (145.5 mg, 248.1 μmol) and 0.2M cesium carbonate (Cs₂CO₃) (372.2 μl, 74.43 μmol) were added. After 4 h at room temperature the reaction mixture was acidified to pH=5 with 10 μl of AcOH. The solvent was removed in vacuo and the residue was purified by preparative HPLC on a C18 column [λ=305 nm; gradient: 0-5 min 5% B; 20-25 min 100% B; 27-35 min 5% B; A=water; B=methanol]. The fractions containing the product were evaporated to 54.46 mg (62%) of HDP 30.1698.

MS (ESI⁺): m/z found: 1425.23 calc.: 1424.6

### Step 2: 6′-[H-Val-Ala-PAB]-α-amanitin (HDP 30.1702)

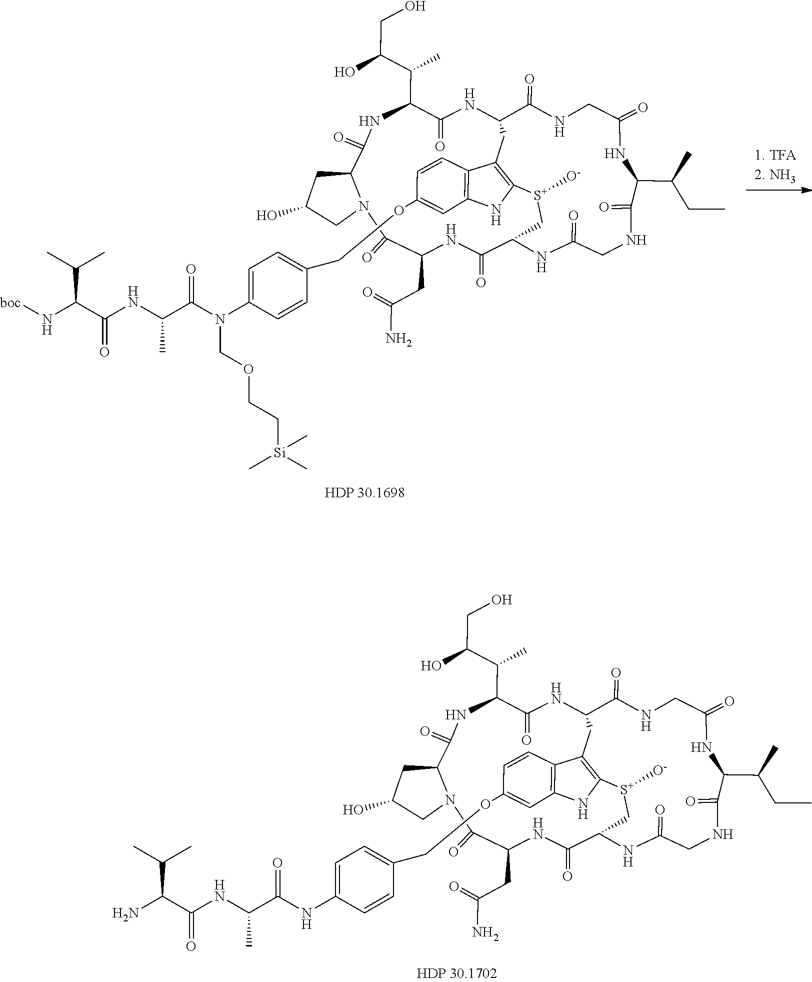

The Boc- and SEM-protected step 5 product (134.29 mg, 94.25 μmol) was dissolved in 5 ml of TFA. After 2 min the mixture was evaporated to dryness at room temperature, redissolved in 5 ml of water, and adjusted to pH 10 with 3.2% ammonia added dropwise. The resulted suspension was freeze-dried, applied to RP18-HPLC [λ=305 nm; gradient: 0-2 min 5% B; 2-10 min 20% B; 10-10.5 min 25% B; 10.5-13 min 100% B; 13-14 min 5% B; A=water with 0.05% TFA; B=acetonitrile] and the pure fractions were evaporated and lyophilized to 68.59 mg (55%) of colorless powder.

MS (ESI+): m/z found: 1194.8 calc.: 1194.53 [M+H]⁺; found: 1217.8 calc.: 1216.51 [M+Na]⁺.

### Step 3: 6′-[(3-maleimidopropanamido)-Val-Ala-PAB]-α-amanitin (HDP 30.1699)

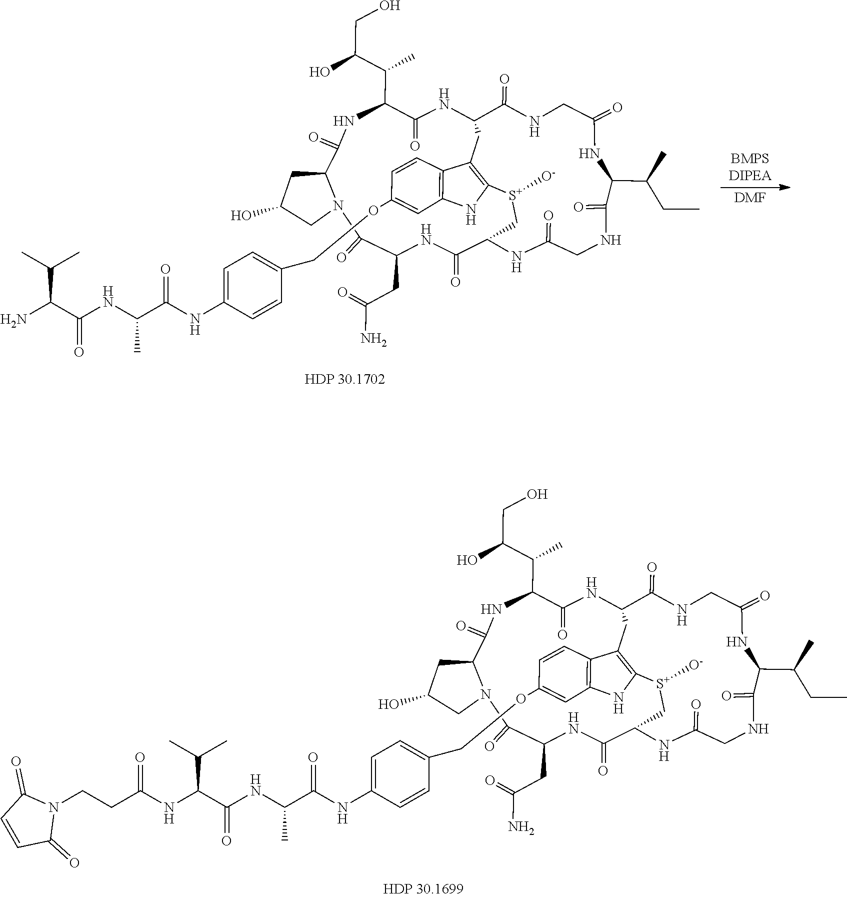

HDP 30.1702 (17.09 mg, 14.3 μmol) was dissolved in dry DMF (350 μl). 3-(maleimido)propanoic acid N-hydroxysuccinimide ester (BMPS) (7.62 mg, 28.6 μmol, 2.0 eq) dissolved in DMF (350 μl), and undiluted DIPEA (9.79 μl, 57.2 μmol, 4.0 eq) were added. After 1 h and 30 minutes of stirring at room temperature under argon, mixture was dripped into 40 ml of cold MTBE and centrifuged at 0° C. The precipitate was collected and washed with 40 ml of MTBE and centrifuged again. The crude product was dried and purified by RP18-HPLC [2=305 nm; gradient: 0-5 min 5% B; 20-25 min 100% B; 27-35 min 5% B; A=water with 0.05% TFA; B=methanol with 0.05% TFA]. The pure fractions were lyophilized to yield 12.51 mg (65%) of title product 6′-[(3-maleidopropanamido)-Val-Ala-PAB]-α-amanitin as white powder.

MS (ESI⁺): m/z found: 1367.50 calc.: 1368.45 [M+Na]⁺.

### B: S-Deoxyamanin (3-maleimidopropanamido)-Val-Ala-p-aminobenzylamide (HDP 30.2115)

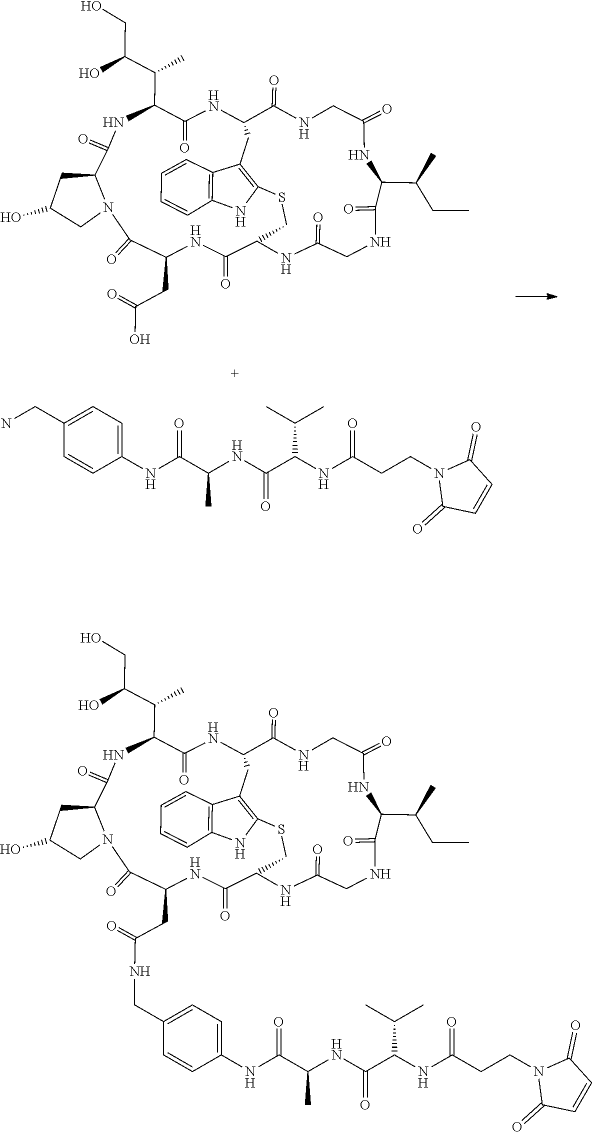

S-Deoxyamanin (15.0 mg, 16.5 μmol) were treated with 429 μl of a 0.1 M solution of (3-maleimidopropanamido)-Val-Ala-p-aminobenzylamine (25.2 μmol, 1.5 eq), 492 μl of 0.1 M TBTU (25.2 μmol, 1.5 eq) and 492 μl of 0.2 M DIEA (49.1 μmol, 3.0 eq) at RT. The reaction was monitored by RP-HPLC. After completion the reaction was quenched with 100 μl H₂O stirred for 15 minutes and injected onto a preparative RP-HPLC.

Yield: 12.2 mg, 56%

Mass spectrometry: 1313.2 [M+H]⁺, 1335.5 [M+Na]⁺

### C: Conjugation of HDP 30.1699 and HDP 30.2115 to Rituximab

For conjugation of maleimide-amatoxin derivatives HDP 30.1699 and HDP 30.2115 to rituximab, stock solutions of the linker toxins were prepared in DMSO at 10 mg/ml. To 4.4 ml antibody solution (9.5 mg/ml in PBS) 44 μl 1 mM EDTA pH 8.0, and 16.7 μl 50 mM TCEP solution (3eqs.) was added and reduction was performed for 2 h at 37° C. The reduced antibodies were split in two 2.2 μl aliquots and treated with 112.5 μl of HDP 30.1699 or 109.8 μl HDP 30.2115 stock solution, respectively. After 30 min shaking at 4° C. remaining thiols were capped by addition of 16.7 μl 100 mM N-ethylmaleimide with shaking for 1 h at room temperature. Subsequently 27.9 μl 100 mM N-acetyl-L-cysteine was added, and shaking was continued for additional 15 min. Amatoxin-ADCs were purified by gelfiltration chromatography using PD-10 columns equilibrated with 1×PBS pH 7.4. Protein containing fractions were dialyzed at 4° C. in Slide-A-Lyzer Dialyse Cassettes (MWCO 20′000) against 4 liter of PBS pH 7.4 overnight. Protein concentration was determined by absorption measurement at 280 nm and adjusted to 5.0 mg/ml, and the samples were sterile filtered (Millex-GV).

TABLE 9
Drug antibody ratios (DAR) of ADCs,
determined by mass spectrometry:

| | | | | Total |
|---|---|---|---|---|
| | DAR | Volume | Conc. | amount |
| Sample | (MS) | [ml] | [mg/ml] | [mg] |
| Rituximab-30.1699 | 3.71 | 5.7 | 5.0 | 28.5 |
| Rituximab-30.2115 | 3.75 | 4.5 | 5.0 | 22.5 |

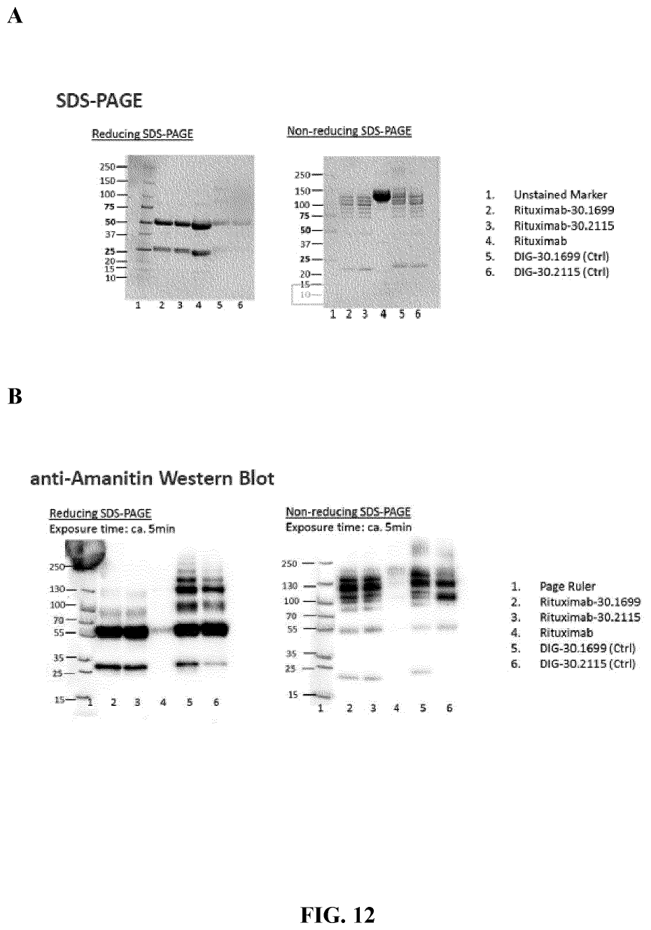

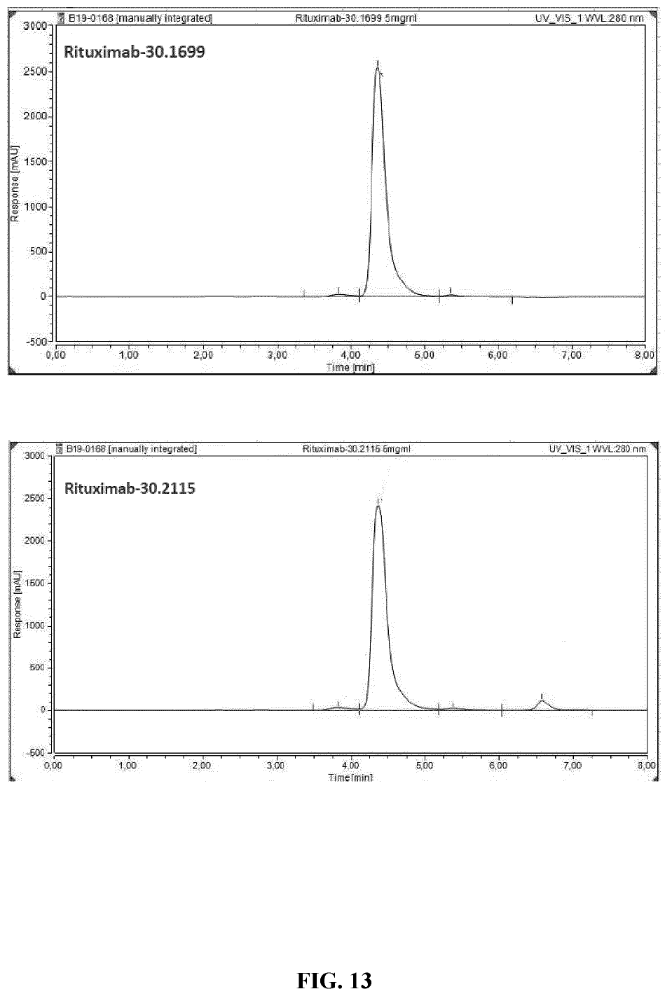

Integrity of the conjugates Rtx-30.1699, comprising an enzymatically cleavable linker connecting one of the natural cysteine residues of rituximab (interchain conjugation) to the 6′-position of the indol system of amino acid 4 of amatoxin, and Rtx-30.2115, comprising an enzymatically cleavable linker connecting one of the natural cysteine residues of rituximab (interchain conjugation) to amino acid 1 of amatoxin, were confirmed by SDS-PAGE analysis and Western blots developed by use of anti-amanitin antibodies. Results are depicted in FIG. 12. The drug/antibody ratio (DAR) of conjugate Rtx-30.1699 was determined to be 3.70, the DAR of conjugate Rtx-30.2115 was determined to be 3.75.

### Example 5.2: Cytotoxicity of Rituximab Amatoxin Conjugates with Enzymatically Cleavable Linkers In Vitro

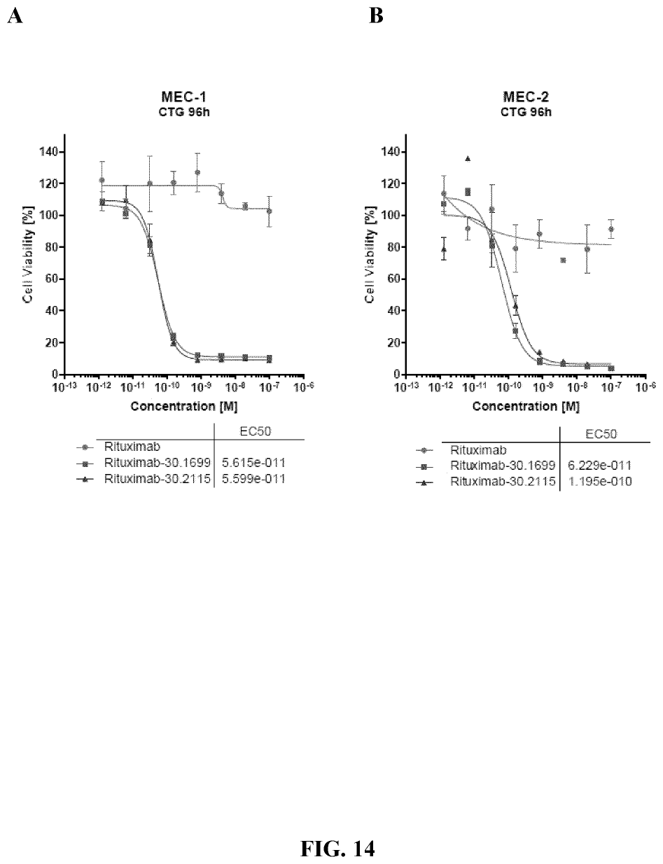

Cytotoxic activities of the rituximab amatoxin conjugates Rtx-30.1699 and Rtx-30.2115 were assessed in vitro on the human chronic B-cell leukemia cell lines MEC-1 and MEC-2, respectively, using a 96-hour CTG assay. Unconjugated rituximab was used as a reference compound. Results are depicted in FIG. 14. Both conjugates induced strong cytotoxic effects on both cell lines, whereas unconjugated rituximab showed no cytotoxic effect at all.

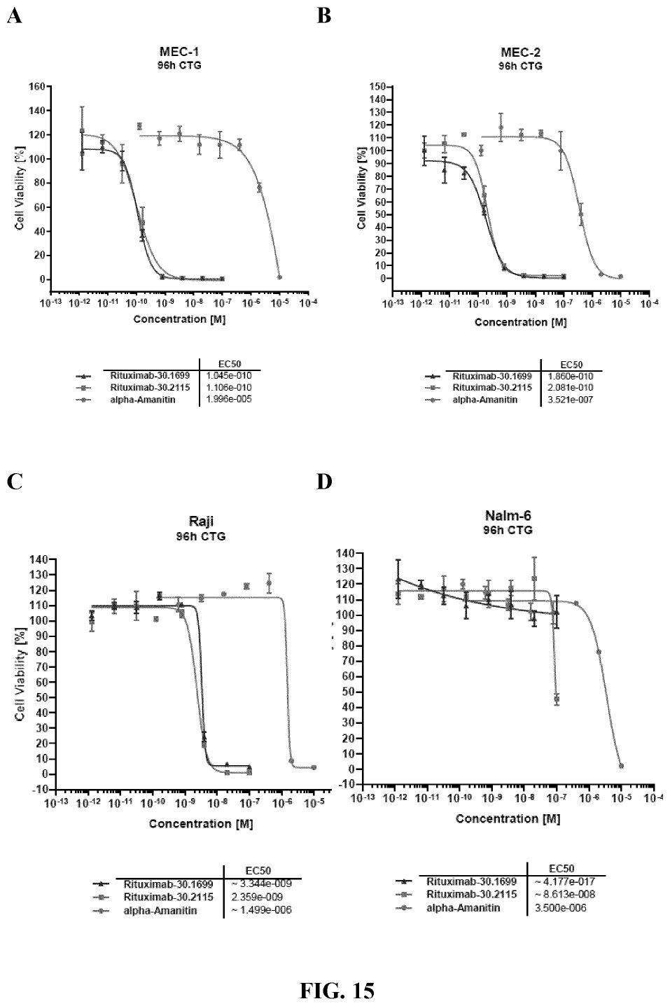

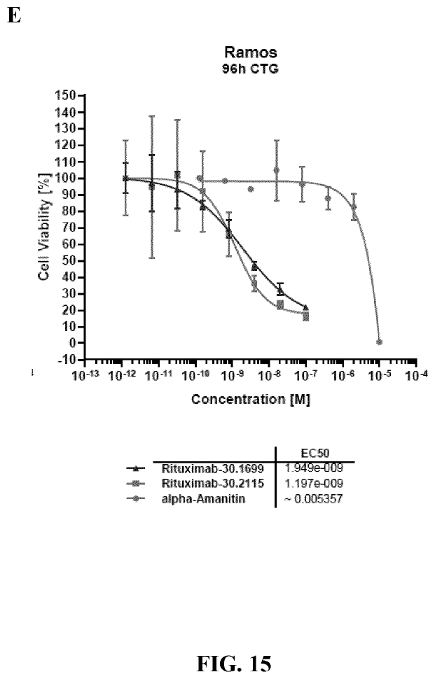

Furthermore, cytotoxic activities of the rituximab amatoxin conjugates Rtx-30.1699 and Rtx-30.2115 were also assessed in vitro on MEC-1, MEC-2, Raji, Nalm-6, and Ramos cell lines, respectively, using a 96-hour CTG assay, in comparison to unconjugated α-amanitin. Results are depicted in FIG. 15. Both conjugates showed strong cytotoxic effects in the low nanomolar range on all cell lines with the exception of Nalm-6 cells which are CD20-negative. Unconjugated α-amanitin in contrast showed cytotoxic effects on all cell lines only in the millimolar range due to unspecific uptake by pinocytosis.

### Example 6: Obinutuzumab Amatoxin Conjugates with Enzymatically Cleavable Linkers

### Example 6.1: Synthesis of Obinutuzumab Amatoxin Conjugates with Enzymatically Cleavable Linkers

By using the methods described in example 5, with the antibody obinutuzumab the following ADCs were obtained:

TABLE 10
Drug antibody ratios (DAR) of ADCs,
determined by mass spectroscopy:

| | | | | Total |
|---|---|---|---|---|
| | | Volume | Conc. | amount |
| Sample | DAR (MS) | [ml] | [mg/ml] | [mg] |
| Obi-30.1699 | 4.64 | 4.1 | 5.0 | 20.5 |
| Obi-30.2115 | 4.33 | 4.2 | 5.0 | 21.0 |

### Example 6.2: Cytotoxicity of Obinutuzumab Amatoxin Conjugates with Enzymatically Cleavable Linkers In Vitro

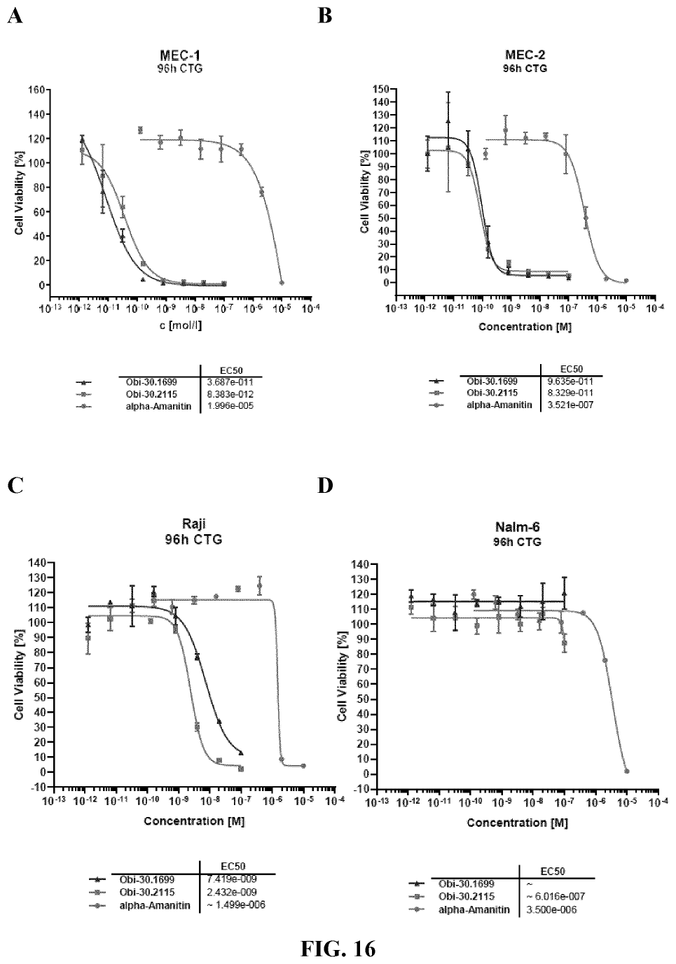

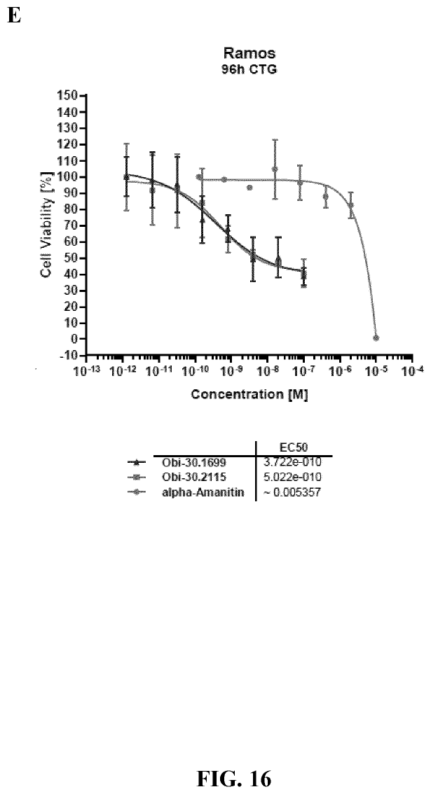

Cytotoxic activities of the obinutuzumab amatoxin conjugates Obi-30.1699 and Obi-30.2115 were assessed in vitro on MEC-1, MEC-2, Raji, Nalm-6, and Ramos cell lines, respectively, using a 96-hour CTG assay, in comparison to unconjugated α-amanitin. Results are depicted in FIG. 16. Both conjugates showed strong cytotoxic effects on all cell lines with the exception of Nalm-6 cells which are CD20-negative. Unconjugated α-amanitin in contrast showed cytotoxic effects on all cell lines only in the millimolar range due to unspecific uptake by pinocytosis.

### Example 7: Cytotoxic Activity of Anti-CD20 Amatoxin Conjugates with Enzymatically Cleavable Linker In Vivo

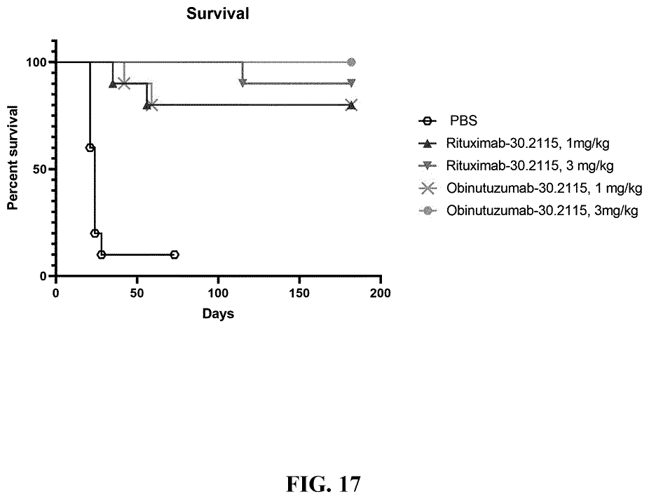

Cytotoxic effects of anti-CD20 amatoxin conjugates Rtx-30.2115 and Obi-30.2115 (see Example 6) in vivo were assessed in a Scid mice xenograft model system. 2.5×10⁶ Raji cells per mouse were i.v. injected into CB17 Scid mice. Rtx-30.2115 and Obi-30.2115 were used for treatment at doses of 1 mg/kg and 3 mg/kg, respectively, each. Results are depicted in FIG. 17.

Over the 52-days study period, both conjugates yielded a 100%-survival rate at a dosage of 3 mg/kg, and a 90%-survival rate at a dosage of 1 mg/kg, in the treated study animals. With the PBS control, in contrast, only 10% of the animals survived longer than 28 days.

Furthermore, the in vivo efficacy of the anti-CD20 amatoxin conjugate Obi-30.1699 was assessed in two patient-derived tumor xenograft models of Richter Syndrome, based on RS9737 and RS1316 cells, respectively. Richter syndrome xenografts based on these cells have been described to be genetically, morphologically, and phenotypically stable and similar to the corresponding primary tumor (Vaisitti et al 2018).

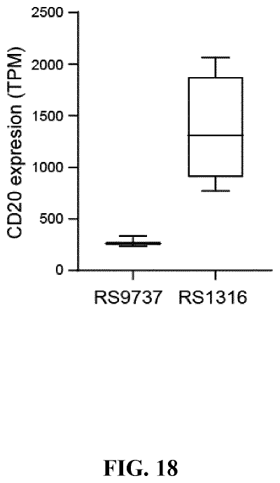

Expression of CD20 in RS9737 and RS1316 cells was assessed by RNA-seq analysis (whole transcriptome shotgun sequencing). Results are depicted in FIG. 18; data are plotted as TPM (transcripts per million). RS9737 cells were shown to express significantly lower level of CD20 than RS1316 cells.

Cell suspensions of patient-derived tumor xenograft RS1316 and RS9737 cells, respectively, were injected into the tail vein in female NOG mice. Animals were treated with a single dose of Obinutuzumab amatoxin conjugate Obi-30.1699 i.v. at day of group allocation (day 21 with RS1316, day 10 with RS9737).

TABLE 11
Study details of patient-derived tumor
xenograft models of Richter Syndrome

| | | | Protein | | | Animals |
|---|---|---|---|---|---|---|
| Group | Compound | DAR | [mg/kg] | Route | Schedule | (n) |
| 1 | PBS (Control) | — | — | i.v. | 1x | 3 |
| 2 | Obi-30.1699 | 4 | 2 | i.v. | 1x | 4 |

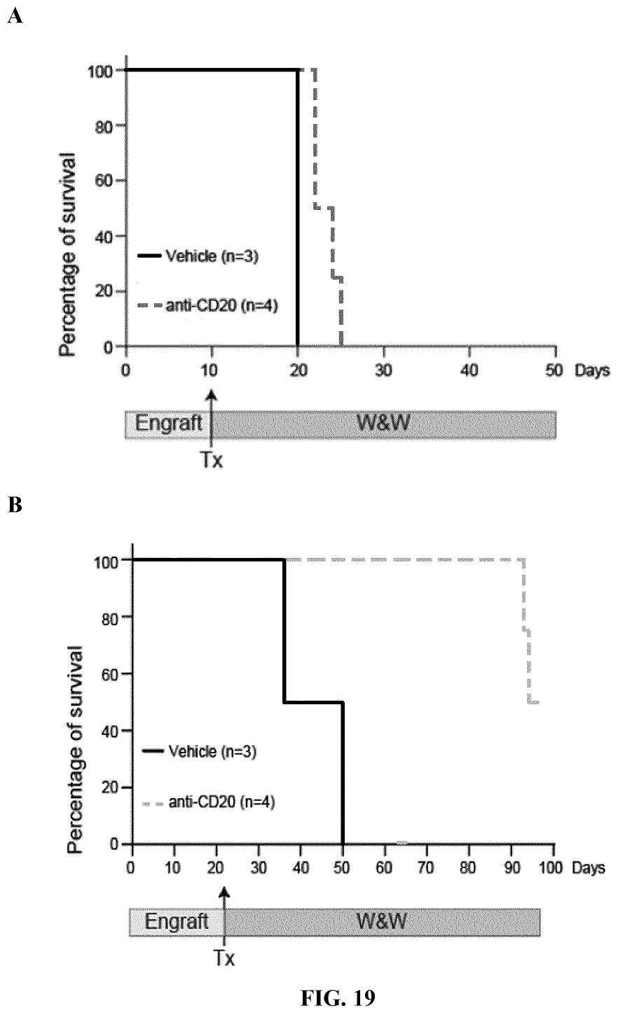

Treatment of mice with the Obinutuzumab amatoxin conjugate Obi-30.1699 had a significant effect on overall survival in both patient-derived tumor xenograft models tested. Results are depicted in FIG. 19. Percentage of survival is shown for the RS9737-based xenograft model (A) and the RS1316-based xenograft model (B) over time. Corresponding to the different CD20 expression levels in RS9737 and RS1316 cells, respectively, a prolonged survival with Obi-30.1699 versus control was observed in the RS9737-based xenograft model (A), and a drastically prolonged survival was observed in the RS1316-based xenograft model (B); in the latter case, 100% of anti-CD20-ADC-treated animals were still alive until day 90 and 50% of anti-CD20-ADC-treated animals were still alive and disease-free (as shown by FACS analysis) at the end of the observation period at day 98.

TABLE 12
Summary overall survival in Richter syndrome xenografts

| Xenograft Model | RS9737 | RS1316 |
|---|---|---|
| Overall Survival | + | +++ |

+++: >100% increase in overall survival (OS); ++: >70% increase in OS; +: >20% increase in OS.

### REFERENCES

Bezombes et al. (2011). Direct Effect of Rituximab in B-Cell-Derived Lymphoid Neoplasias: Mechanism, Regulation, and Perspectives. Mol. Cancer Res. Vol 9(11): 1435-1442.
Boross P and Leusen H J W (2012). Mechanisms of action of CD20 antibodies. American Journal of Cancer Research Vol. 2(6): 676-690.
DiJoseph J F et al. (2007). CD20-specific antibody-targeted chemotherapy of non-Hodgkin's B-cell lymphoma using calicheamicin-conjugated rituximab. Cancer Immunol Immunother. Vol. 56:1107-1117.
Dransfield I. (2014). Inhibitory FcγRIIb and CD20 internalization. Blood Vol. 123(5): 606-607.
Edelman et al. Proc. Natl. Acad. Sci. USA 1969, 63, 78-85).
Falchi L et al. (2018). An Evidence-based Review of Anti-CD20 Antibody-containing Regimens for the Treatment of Patients With Relapsed or Refractory Chronic Lymphocytic Leukemia, Diffuse Large B-cell Lymphoma, or Follicular Lymphoma. Clinical Lymphoma, Myeloma & Leukemia, Vol. 18(8): 508-518.
Polson A G (2013). Antibody-Drug Conjugates for the Treatment of B-Cell Malignancies. In: Phillips G L (ed.) (2013). Antibody-Drug Conjugates and Immunotoxins: From Pre-Clinical Development to Therapeutic Applications, Cancer Drug Discovery and Development. Springer Science+Business Media New York, pp. 139-147.
Polson A G et al. (2009). Antibody-Drug Conjugates for the Treatment of Non-Hodgkin's Lymphoma: Target and Linker-Drug Selection. Cancer Res. Vol. 69(6): 2358-2364.
Kim E G and Kim K M (2015). Strategies and Advancement in Antibody-Drug Conjugate Optimization for Targeted Cancer Therapeutics. Biomol. Ther. Vol. 23(6): 493-509.
Kohler and Milstein, Nature 256:495 (1975); Eur. J. Immunol. 6:511 (1976).
Lambert J M et al. (1985). Purified Immunotoxins That Are Reactive with Human Lymphoid Cells. J. Biol. Chem. Vol. 260:12035-12041.
Law, C-L et al. (2004). Efficient Elimination of B-Lineage Lymphomas by Anti-CD20-Auristatin Conjugates. Clinical Cancer Research Vol. 10:7842-7851.
Press et al. (1989). Endocytosis and Degradation of Monoclonal Antibodies Targeting Human B-Cell Malignancies. Cancer Research Vol. 49:4906-4912.
Staudacher A H and Brown M P (2017). Antibody drug conjugates and bystander killing: is antigen-dependent internalisation required? British Journal of Cancer Vol. 117:1736-1742.
Taylor R P and Lindorfer M A (2008). Immunotherapeutic mechanisms of anti-CD20 monoclonal antibodies. Current Opinion in Immunology Vol. 20:444-449.
Vaisitti et al. (2018). Novel Richter Syndrome Xenograft Models to Study Genetic Architecture, Biology, and Therapy Responses. Cancer Research Vol. 78:3413-3420. Vangeepuram et al. (1997). Processing of Antibodies Bound to B-Cell Lymphomas and Lymphoblastoid Cell Lines. Cancer Vol. 80:2425-2430.
Weiner G J (2010). Rituximab: Mechanism of Action. Semin Hematol. Vol. 47:115-123.
Winiarska et al. (2007). CD20 as a target for therapy. Centr. Eur. J. Immunol. Vol. 32(4): 239-246.

1. A conjugate comprising (i) a target binding moiety, (ii) at least one toxin, and (iii) optionally at least one linker connecting said target binding moiety with said at least one toxin, wherein said target binding moiety binds to CD20 and wherein said at least one toxin is an amatoxin, wherein said target binding moiety is selected from the group consisting of (i) an antibody, (ii) an antigen-binding fragment thereof, (iii) an antigen-binding derivative thereof, and (iv) an antibody-like protein, each binding to CD20, respectively.

2. The conjugate according to claim 1, wherein said target binding moiety is selected from the group consisting of
(i) a monoclonal antibody,
(ii) a variable domain (Fv), a Fab fragment or an F(ab)₂ fragment,
(iii) a single-chain Fv (scFv), and
(iv) an antibody-like protein,
each binding to CD20, respectively.

3. The conjugate according to claim 1, wherein said antibody, or antigen-binding fragment thereof or antigen-binding derivative thereof, is a murine, a chimeric, a humanized or a human antibody, or antigen-binding fragment or antigen-binding derivative thereof, respectively.

4. The conjugate according to claim 1, wherein said antibody, or antigen-binding fragment thereof, or antigen-binding derivative thereof, respectively, is selected from the group consisting of rituximab, obinutuzumab, ibritumomab, tositumomab, ofatumumab, ocrelizumab, and ublituximab.

5. The conjugate according to claim 1, wherein said antibody has been genetically engineered to comprise a heavy chain 118Cys, a heavy chain 239Cys, or heavy chain 265Cys according to the EU numbering system, and wherein said linker, if present, or said amatoxin is connected to said antibody via said heavy chain 118Cys, or said heavy chain 239Cys, or heavy chain 265Cys residue, respectively.

6. The conjugate according to claim 1, wherein said linker, if present, or said amatoxin is connected to said antibody via any of the natural Cys residues of said antibody.

7. The conjugate according to claim 4, wherein said antibody is rituximab or rituximab that has been genetically engineered to comprise a heavy chain 265Cys according to the EU numbering system.

8. The conjugate according to claim 4, wherein said antibody is rituximab and wherein the said linker, if present, or said amatoxin is connected to rituximab via any of the naturally occurring Cys residues which form the interchain disulfide bonds of rituximab.

9. The conjugate according to claim 1, wherein said linker is a non-cleavable or a cleavable linker.

10. The conjugate according to claim 9, wherein said cleavable linker is selected from the group consisting of an enzymatically cleavable linker and a chemically cleavable linker.

11. The conjugate according to claim 1, wherein said amatoxin comprises (i) an amino acid 4 with a 6′-deoxy position and (ii) an amino acid 8 with an S-deoxy position.

12. The conjugate according to claim 1, wherein said linker, if present, or said target binding moiety is connected to said amatoxin via (i) the γC-atom of amatoxin amino acid 1, or (ii) the δ C-atom of amatoxin amino acid 3, or (iii) the 6′-C-atom of amatoxin amino acid 4.

13. The conjugate according to claim 1, wherein said conjugate comprises any of the following compounds of formulas (I) to (XII), respectively, as linker-amatoxin moieties:

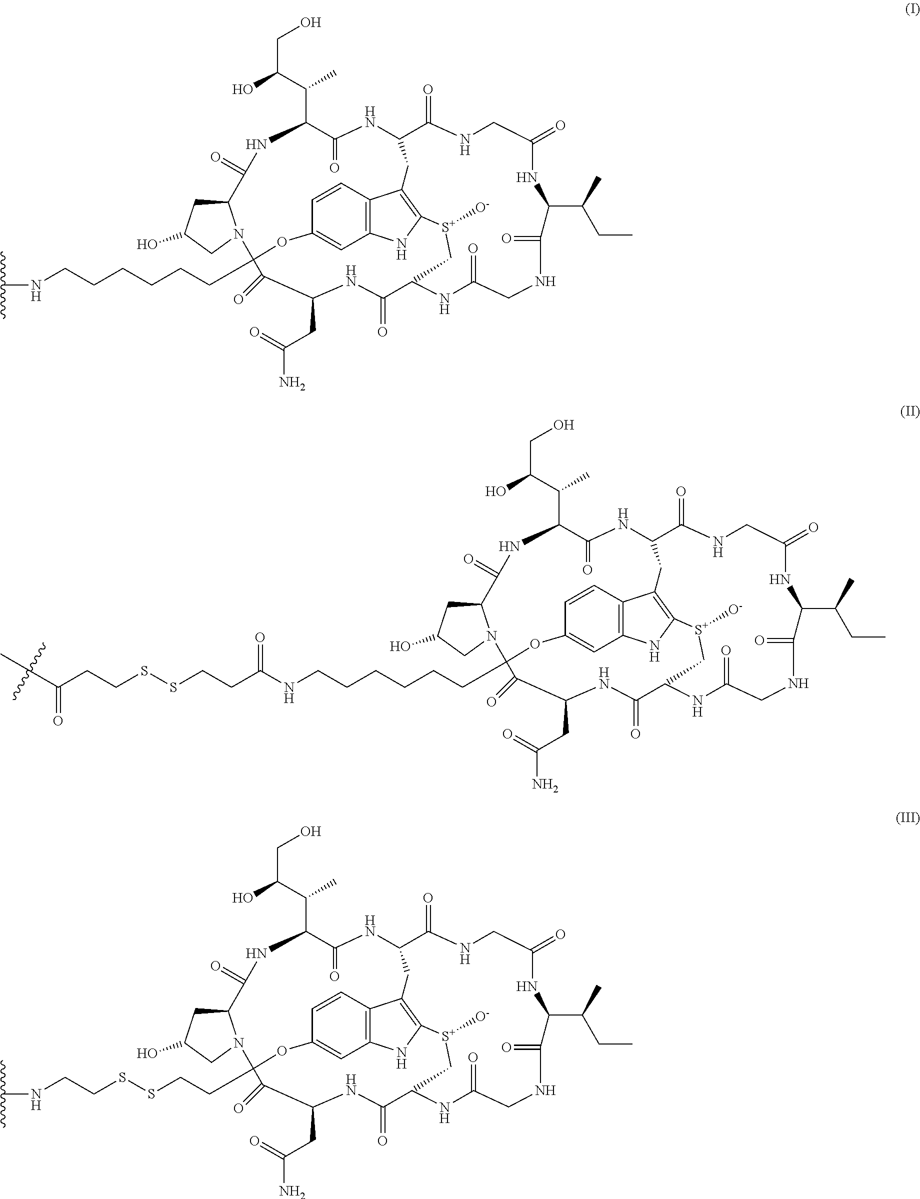

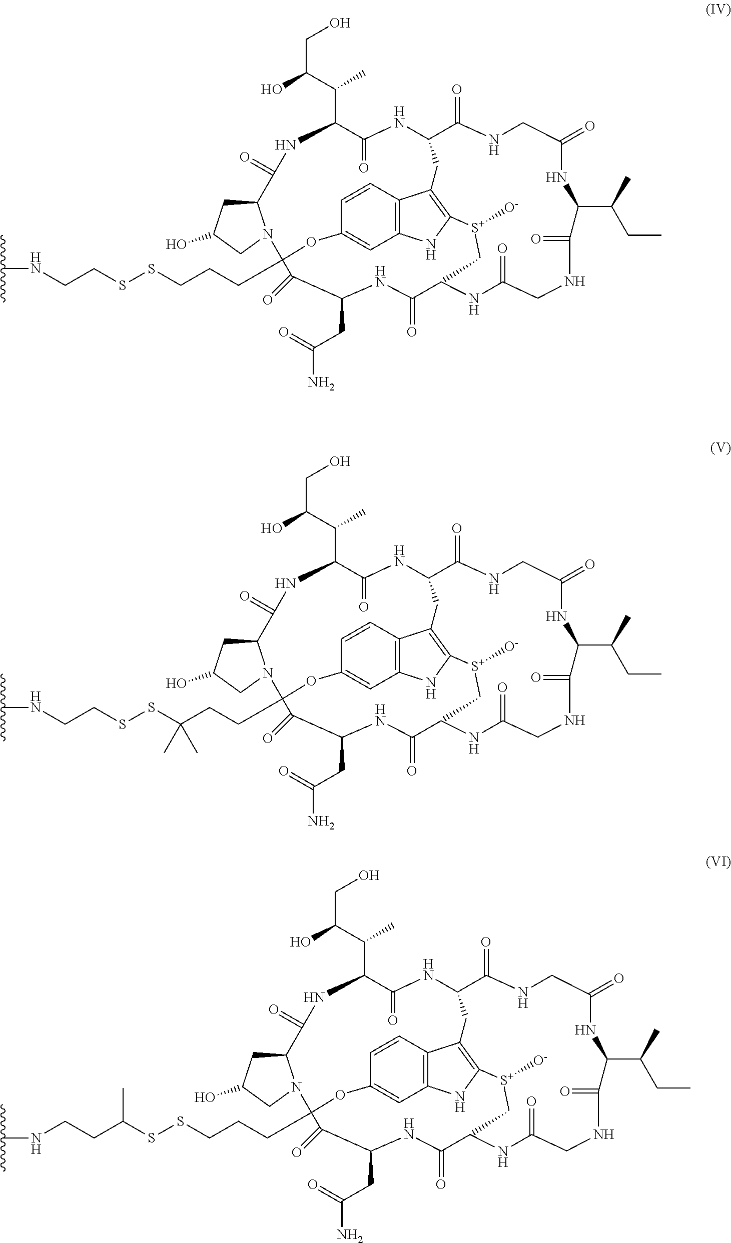

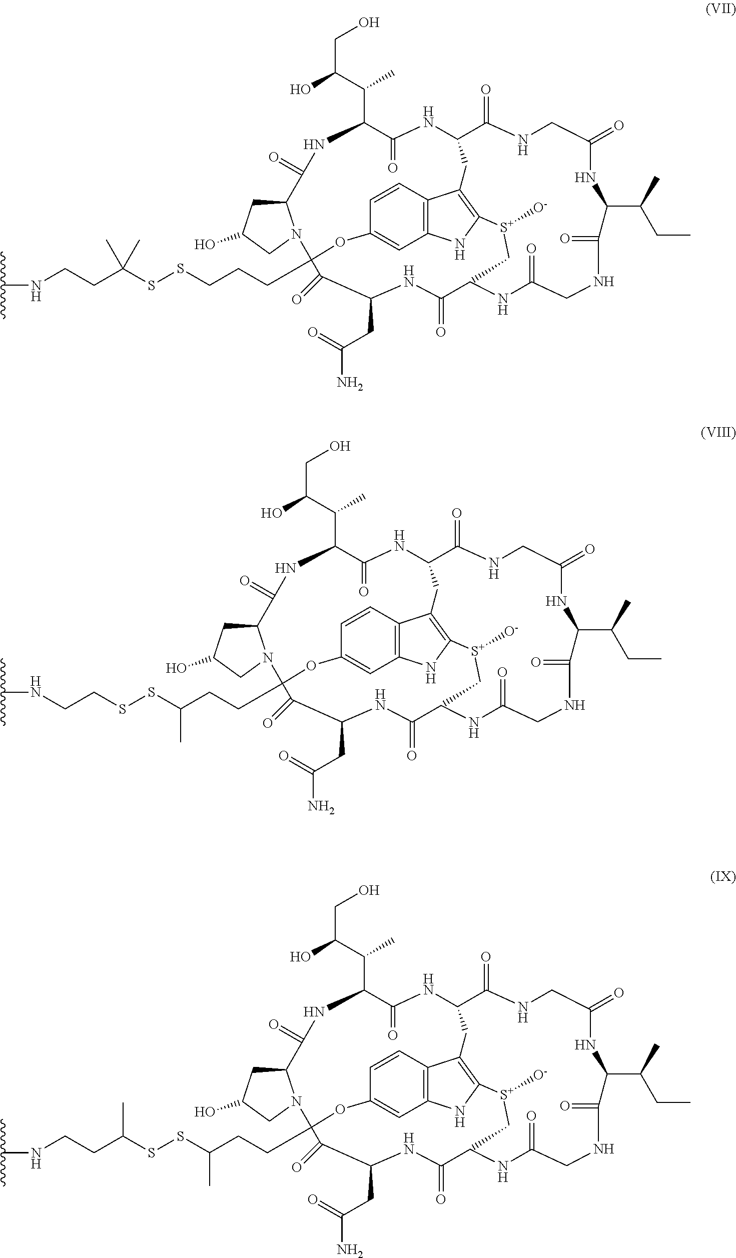

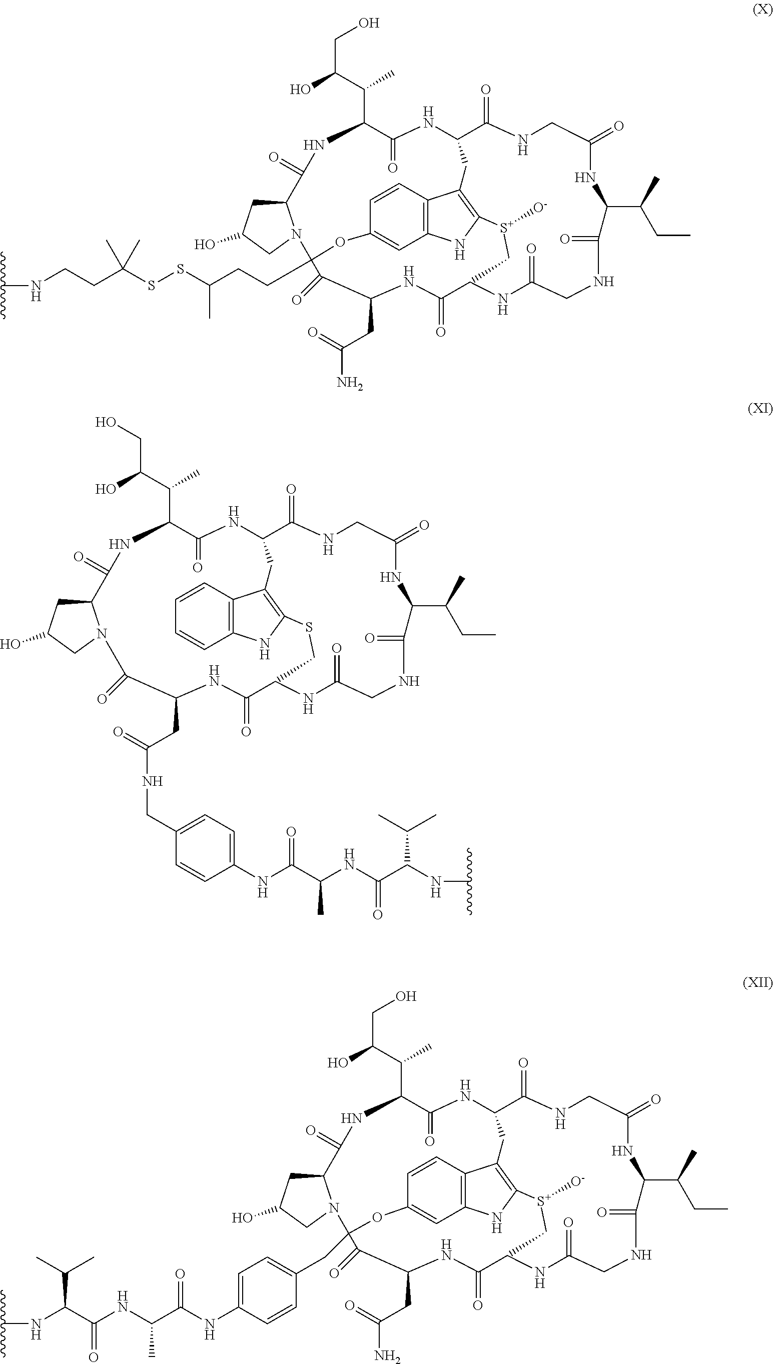

14. The conjugate according to claim 1, wherein said conjugate comprises an antibody as target binding moiety conjugated to amatoxin linker moieties according to any one of formula XIII to XXII 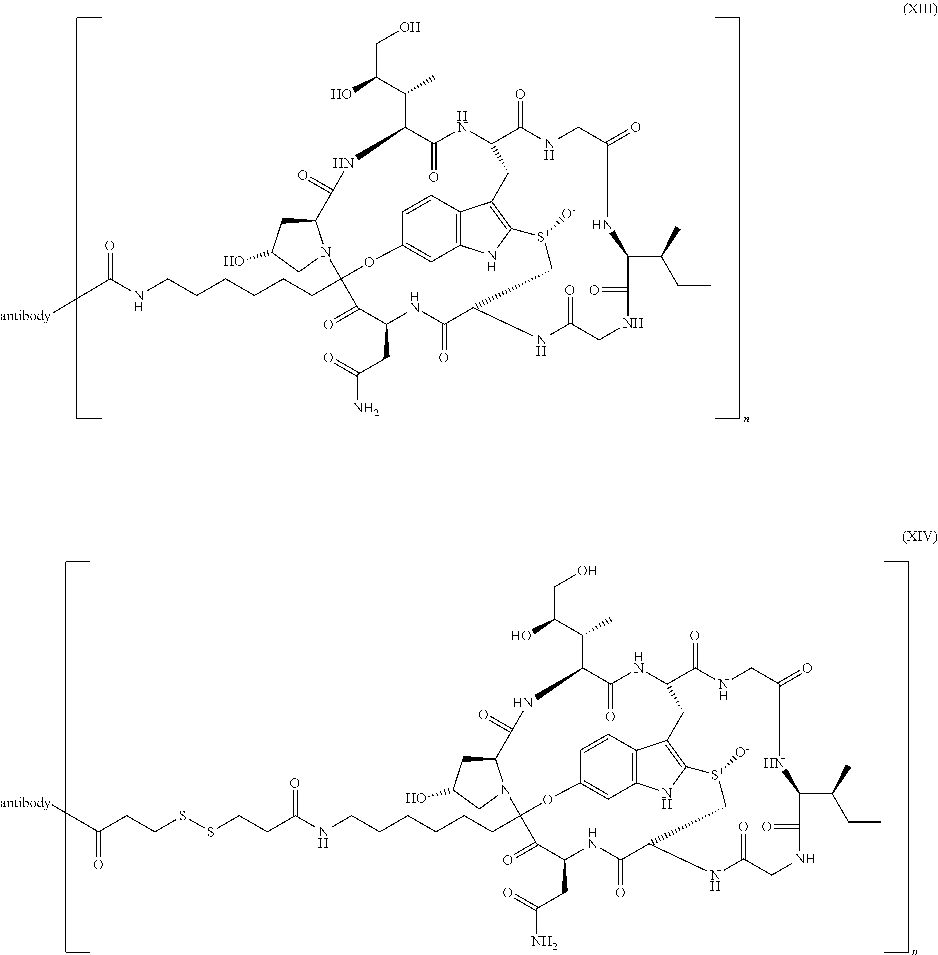 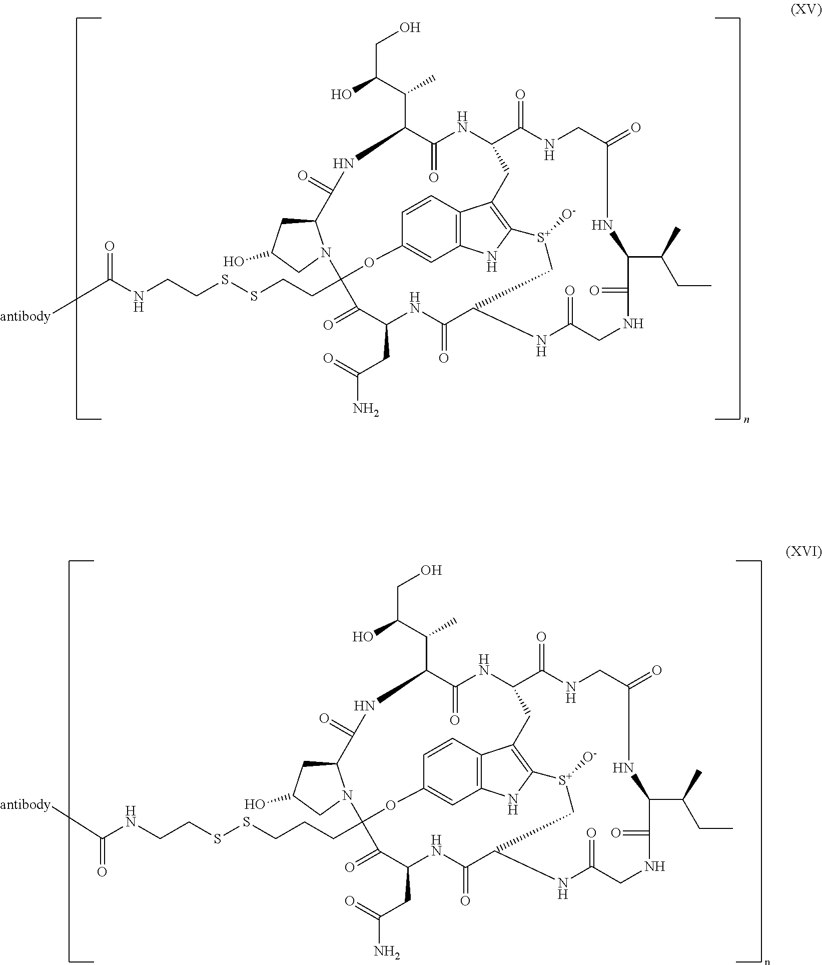 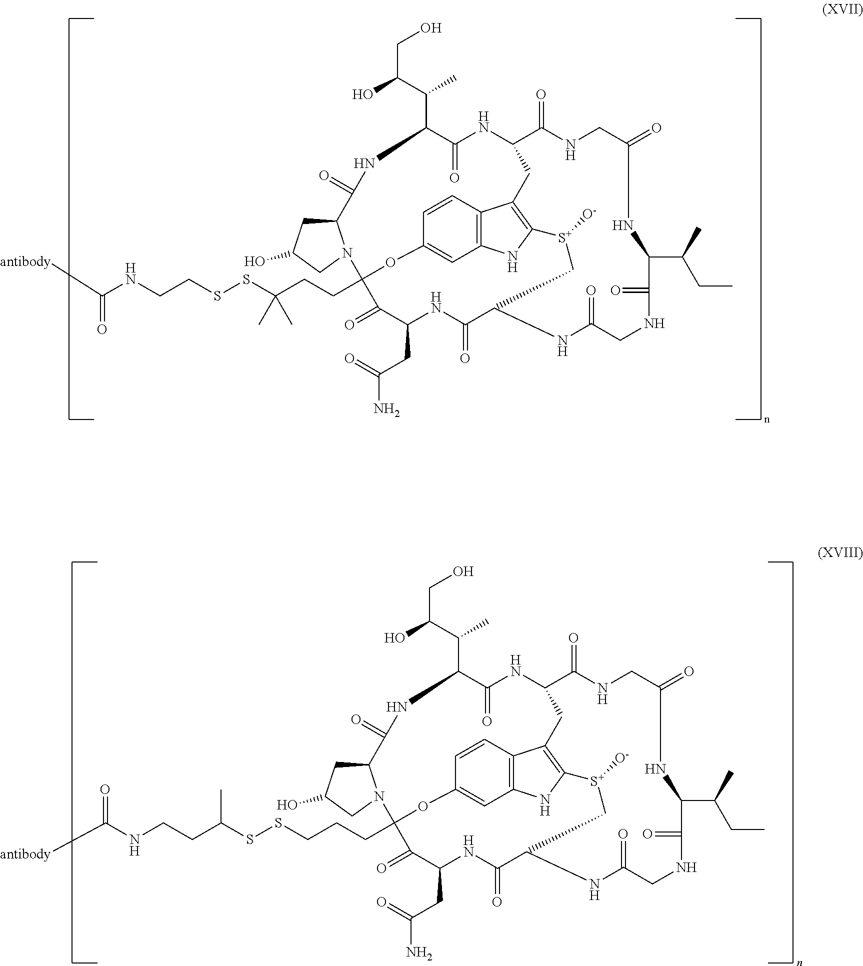 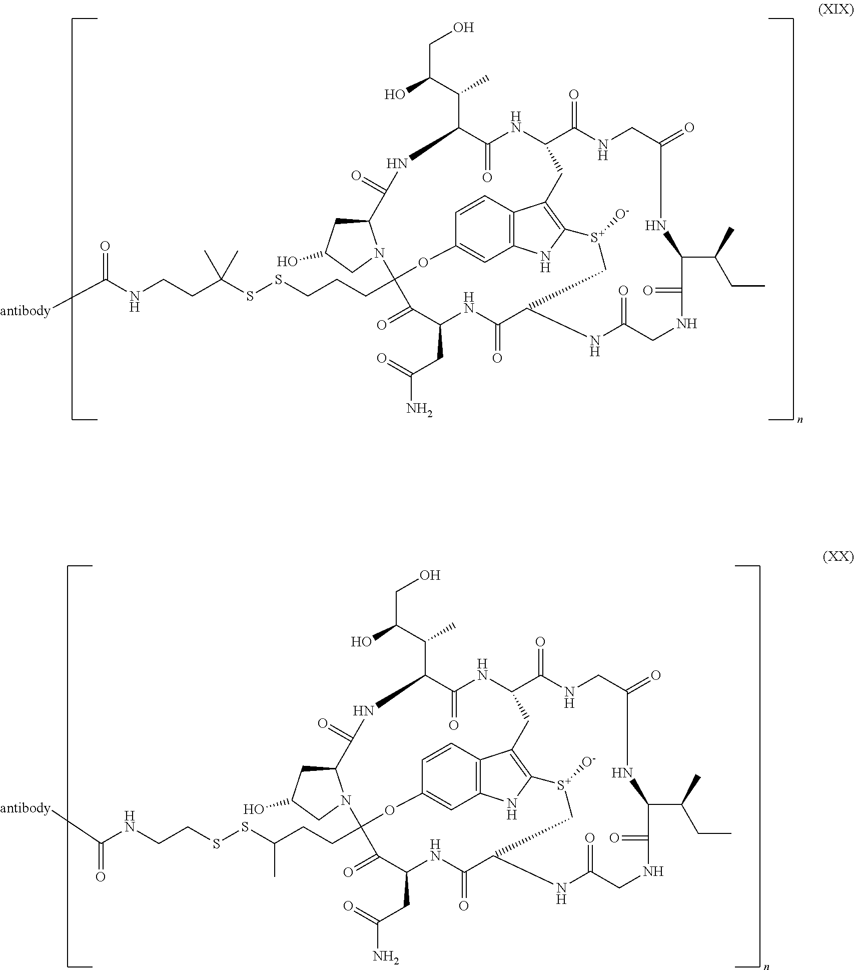 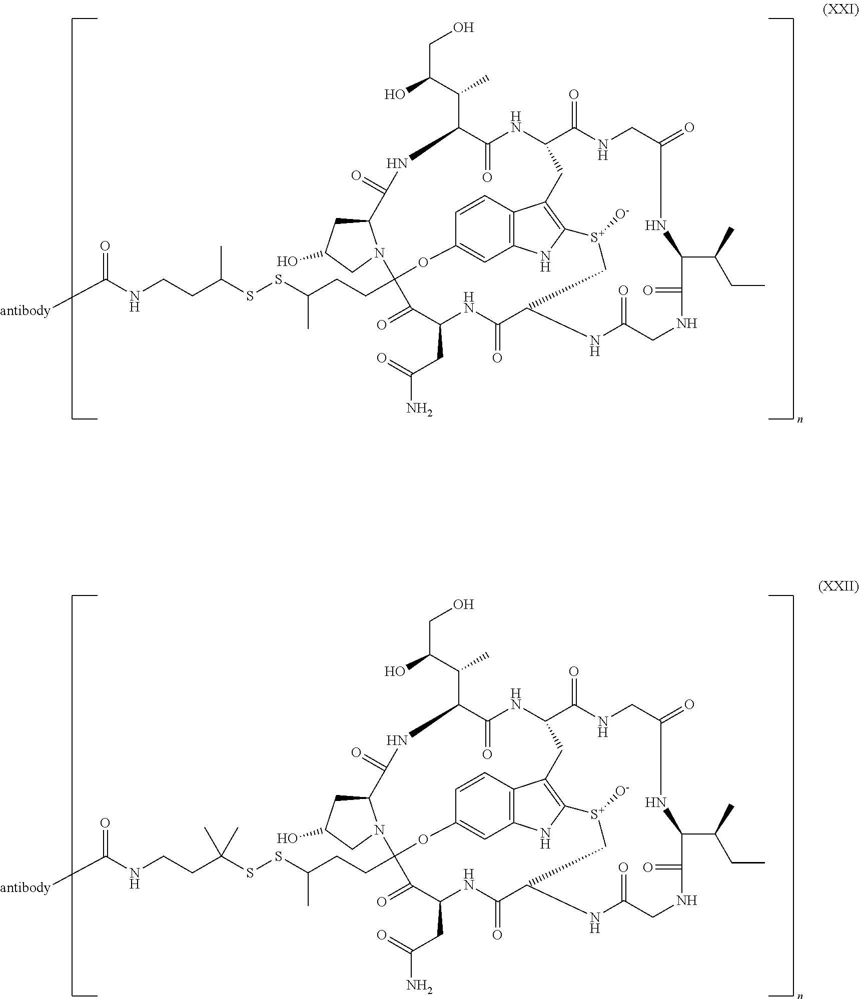
wherein said amatoxin linker moieties are coupled to ε-amino groups of naturally occurring lysine residues of said antibody, and wherein n is preferably from 1 to 7.

15. The conjugate according to claim 1, wherein said conjugate comprises an antibody as target binding moiety conjugated to amatoxin linker moieties according to any one of formula XXIII to XXIV 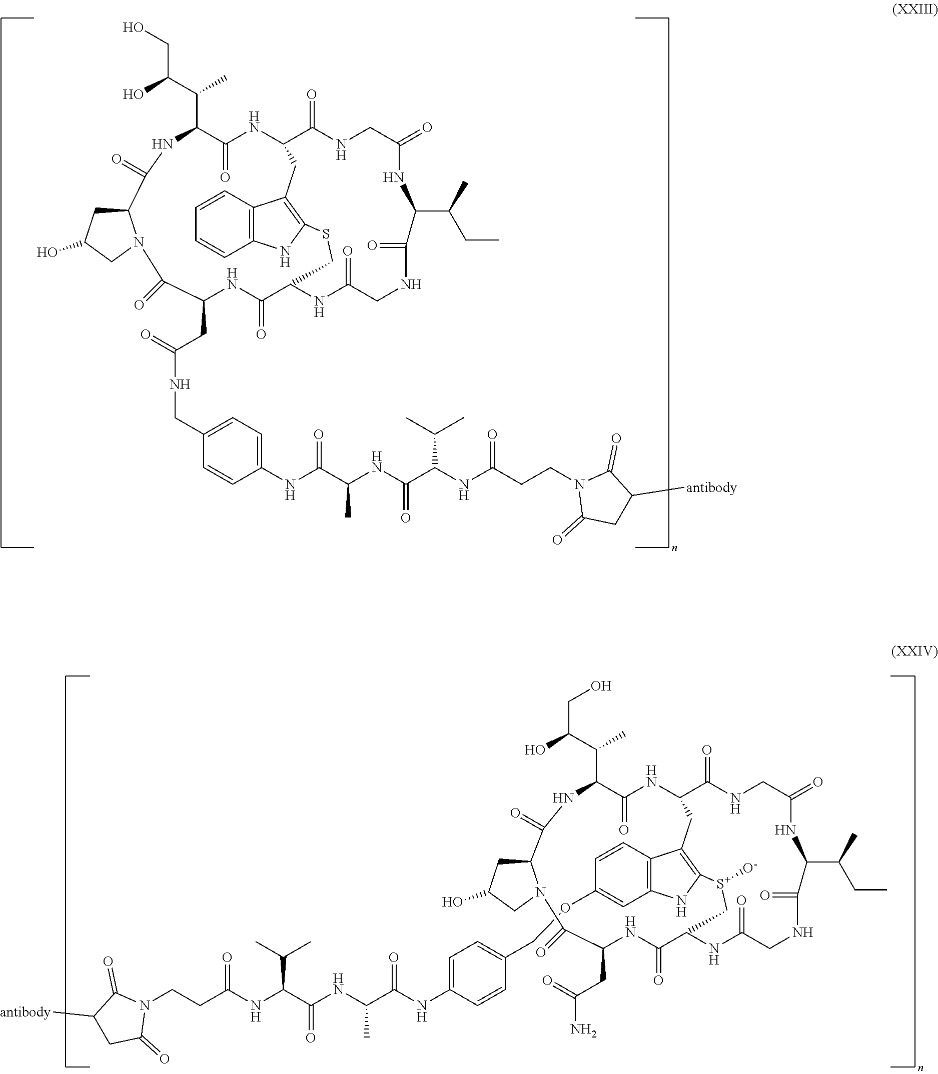
wherein said amatoxin linker moieties are coupled to the thiol groups of cysteine residues of the antibody, and wherein n is from 1 to 7.

16. The conjugate according to claim 1, wherein said conjugate is selected from the group consisting of
(i) a conjugate comprising the antibody rituximab as target binding moiety conjugated to at least one amatoxin-linker moiety of formula (XI) via thioether linkage to at least one naturally occurring Cys residue of rituximab, according to formula XXV 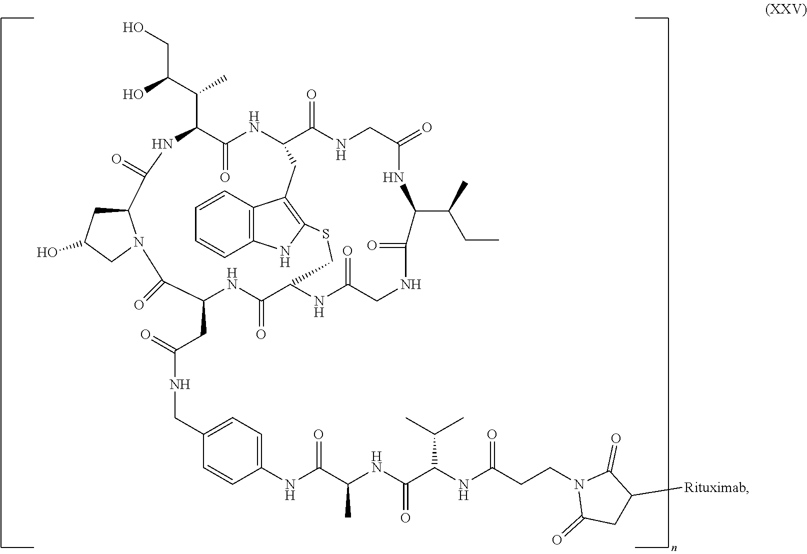
(ii) a conjugate comprising the antibody rituximab genetically engineered to comprise a heavy chain 265Cys according to the EU numbering system as target binding moiety conjugated to an amatoxin linker moiety of formula (XI) via thioether linkage to said heavy chain 265Cys residue of said genetically engineered rituximab, according to formula XXVI 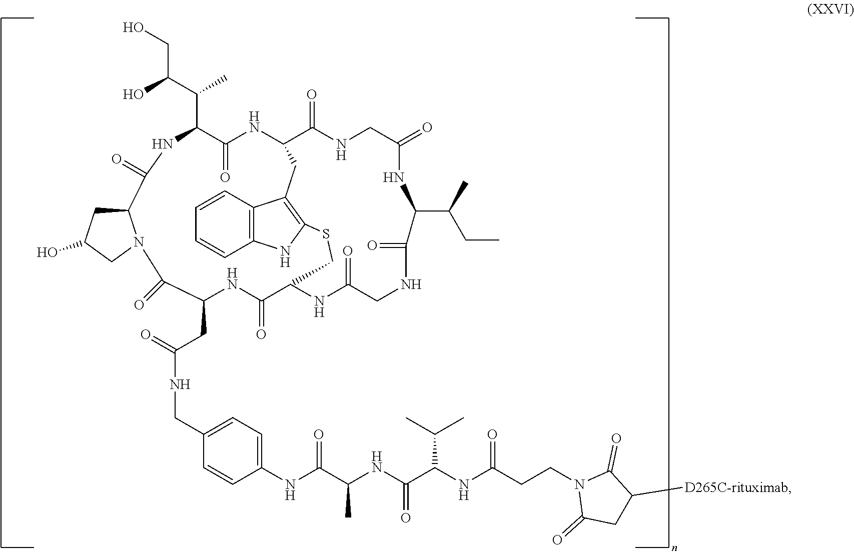
(iii) a conjugate comprising the antibody rituximab as target binding moiety conjugated to at least one amatoxin-linker moiety of formula (XII) via thioether linkage to at least one naturally occurring Cys residue of rituximab, according to formula XXVII 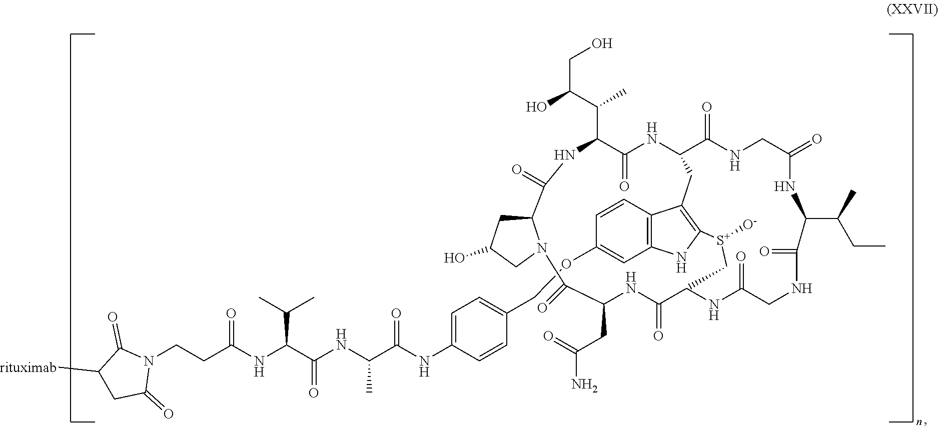
and
(iv) a conjugate comprising the antibody rituximab genetically engineered to comprise a heavy chain 265Cys according to the EU numbering system as target binding moiety conjugated to an amatoxin linker moiety of formula (XII) via thioether linkage to said heavy chain 265Cys residue of said genetically engineered rituximab, according to formula XXVIII 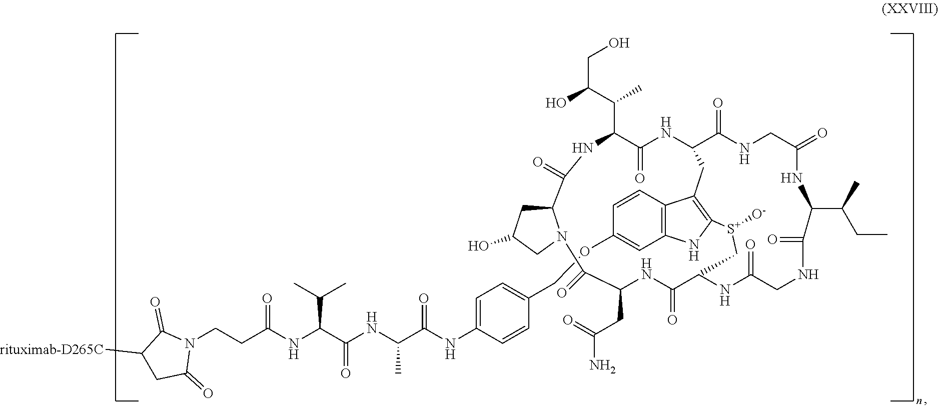
wherein n is 1 to 7 for (i), (iii), and n is 1 to 2 for (ii), (iv).

17. A pharmaceutical composition comprising the conjugate of claim 1.

18.-20. (canceled)

21. A method of treating a patient suffering from a B lymphocyte-associated malignancy or B cell-mediated autoimmune disease, comprising administering an effective amount of a conjugate according to claim 1 or a pharmaceutical composition comprising said conjugate to the patient.

22. The method of treating a patient according to claim 21, wherein the B lymphocyte-associated malignancy or B cell-mediated autoimmune disease is selected from the group consisting of non-Hodgkin's lymphoma, follicular lymphoma, diffuse large B cell non-Hodgkin's lymphoma, chronic lymphocytic leukaemia, Richter syndrome, rheumatoid arthritis, granulomatosis with polyangiitis and microscopic polyangiitis, and pemphigus vulgaris.


SEQUENCE LISTING (6 sequences)

SEQ ID NO: 1 - protein, 213 aa, Artificial Sequence
  Rituximab Light Chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Artificial Sequence
  Rituximab Heavy Chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 451 aa, Artificial Sequence
  Rituximab 265Cys Heavy Chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 451 aa, Artificial Sequence
  Rituximab 118Cys Heavy Chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSACSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 451 aa, Artificial Sequence
  Rituximab 239Cys Heavy Chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP